(19) United States
(12) Patent Application Publication
(10) Pub. No.: US 2023/0272376 A1
(43) Pub. Date: Aug. 31, 2023

(54) METHODS AND COMPOSITIONS FOR THE ADAR-MEDIATED EDITING OF METHYL-CPG BINDING PROTEIN 2 (MECP2)

(71) Applicant: Korro Bio, Inc., Cambridge, MA (US)

(72) Inventors: Matthew Jarpe, Quincy, MA (US); Mallikarjuna Reddy Putta, Lexington, MA (US); Andrew Fraley, Arlington, MA (US)

(21) Appl. No.: 17/985,970

(22) Filed: Nov. 14, 2022

Related U.S. Application Data
(63) Continuation of application No. PCT/US2021/032154, filed on May 13, 2021.
(60) Provisional application No. 63/025,361, filed on May 15, 2020.

(51) Int. Cl. C12N 15/11; C12N 9/78; C12N 15/85; C12N 15/90

(52) U.S. Cl. CPC C12N 15/11; C12Y 305/04004; C12N 9/78; C12N 15/85; C12N 15/90; C12N 2310/321; C12N 2310/315; C12N 2320/34; C12N 2800/107

(57) ABSTRACT

The present invention relates to methods and compositions for editing a MECP2 polynucleotide, e.g., a MECP2 polynucleotide comprising a SNP associated with Rett syndrome. The invention also relates to methods and compositions for treating or preventing Rett syndrome in a subject.

### RELATED APPLICATIONS

This application is a 35 § U.S.C. 111(a) continuation application which claims the benefit of priority to PCT/US2021/032154, filed on May 13, 2021, which in turn claims the benefit of priority to U.S. Provisional Application No. 63/025,361, filed on May 15, 2020. The entire contents of each of the foregoing applications are incorporated herein by reference.

### SEQUENCE LISTING

The instant application contains a Sequence Listing which has been submitted electronically in XML file format and is hereby incorporated by reference in its entirety. Said XML copy, created on Jan. 18, 2023, is named 131522_00902_SL.XML and is 6,201,047 bytes in size.

### BACKGROUND OF THE INVENTION

Rett syndrome is a progressive neurodevelopmental disorder and one of the most common causes of mental retardation in females, with an incidence of 1 in 10-15,000 (Hagberg, Acta Paediatr Scand. 1985 May; 74(3):405-8). Patients with Rett syndrome appear to develop normally until 6-18 months of age, then gradually lose speech and purposeful hand use, and develop microcephaly, seizures, autism, ataxia, intermittent hyperventilation and stereotypic hand movements (Hagberg et al., Ann Neurol. 1983 October; 14(4):471-9). Other features such as ataxia, gait apraxia, seizures, breathing dysrhythmias (apnea or hyperpnea), and autistic behaviour may also emerge. They also suffer decreased somatic growth and wasting. Following this period of rapid deterioration, patients stabilize and then may recover some skills. Over 90% of cases of Rett syndrome are caused by a mutation in the MECP2 gene.

MECP2 is an abundant chromosome-binding protein that selectively binds 5-methyl cytosine residues in symmetrically positioned CpG dinucleotides in mammalian genomes (Lewis et al., Cell. 1992 Jun. 12; 69(6):905-14). These residues are preferentially located in the promoter regions of genes that are subject to transcriptional silencing after DNA methylation. MECP2 is the molecular link between DNA methylation and transcriptional silencing by histone deacetylation (Nan et al., Nature. 1998 May 28; 393(6683):386-9.). It contains at least two functional domains: an 85 amino acid methyl-CpG-binding domain, essential for its binding to 5-methyl cytosine (Nan et al., Nucleic Acids Res. 1993 Oct. 25; 21(21):4886-92), and a 104 amino acid transcriptional repression domain that interacts with histone deacetylase and the transcriptional corepressor Sin3A. Interactions between this transcription repressor complex and chromatin-bound MECP2 leads to deacetylation of core histones, which in turn leads to transcriptional repression (Nan et al., 1998). Many of the genes that are known to be regulated by the MECP2 protein play a role in normal brain function, particularly the maintenance of synapses.

While treatments are available for alleviating the symptoms of Rett syndrome, there is no known cure (National Institute of Neurological Disorders and Stroke; October 2017). Accordingly, there exists an ongoing need for novel methods that can selectively and efficiently edit the MECP2 gene, and correct any pathogenic mutations in the gene in order to treat and/or prevent Rett syndrome.

### SUMMARY OF THE INVENTION

The present invention provides methods and compositions for editing a MECP2 polynucleotide and methods of treating or preventing a MECP2-associated disease, in a subject using a guide oligonucleotide capable of effecting an adenosine deaminase acting on RNA (ADAR)-mediated adenosine to inosine alteration in the target gene.

The present invention provides methods for the site specific editing of MECP2 in a cell, without the need to transduce or transfect the cell with genetically engineered editing enzymes. The design of the guide oligonucleotides of the present invention allows the recruitment of the ADAR enzyme, to the specific editing sites disclosed herein. The methods of the present invention can conveniently be used to make changes in MECP2, for example to reverse mutations that are involved in, or cause, MECP2-associated disease, thereby alleviating the symptoms of the disease. This is of great advantage when used in treating the MECP2-associated disease, e.g., Rett syndrome. Further, the guide oligonucleotides used in the methods of the present invention provide an ease of delivery and avoid any immune response, e.g., associated with viral vectors. In addition, editing of the MECP2 mRNA preserves the endogenous expression level, transcriptional control, and splice variants of the wild type gene. This is particularly important for the MECP2 gene because over and under expression of the gene can both cause a deleterious phenotype.

The invention provides, in one aspect, a method of editing an MECP2 polynucleotide comprising a single nucleotide polymorphism (SNP) associated with Rett syndrome. The method comprises contacting the MECP2 polynucleotide with a guide oligonucleotide capable of effecting an adenosine deaminase acting on RNA (ADAR)-mediated adenosine to inosine alteration of the SNP associated with Rett syndrome, thereby editing the MECP2 polynucleotide.

In some embodiments, the MECP2 polynucleotide is contacted with the guide oligonucleotide in a cell. In some embodiments, the cell endogenously expresses ADAR. In some embodiments, the ADAR is a human ADAR. In some embodiments, the ADAR is human ADAR1. In other embodiments, the ADAR is human ADAR2.

In some embodiments, the cell is selected from eukaryotic cell, a mammalian cell, and a human cell. In some embodiments, the cell is in vivo. In other embodiments, the cell is ex vivo.

In one aspect, the present invention provides a method of treating Rett syndrome in a subject in need thereof. The method comprises identifying a subject with a single nucleotide polymorphism (SNP) associated with Rett syndrome in an MECP2 polynucleotide; contacting the MECP2 polynucleotide in a cell of the subject with a guide oligonucleotide capable of effecting an adenosine deaminase acting on RNA (ADAR)-mediated adenosine to inosine alteration of the SNP associated with Rett syndrome, thereby treating the subject.

In another aspect, the present invention provides a method of treating Rett syndrome in a subject in need thereof. The method comprising identifying a subject with a single nucleotide polymorphism (SNP) associated with Rett syndrome in an MECP2 polynucleotide; contacting the MECP2 polynucleotide in a cell with a guide oligonucleotide capable of effecting an adenosine deaminase acting on RNA (ADAR)-mediated adenosine to inosine alteration of the SNP associated with Rett syndrome, and administering the cell to the subject, thereby treating the subject.

In some embodiments, the cell is autologous, allogenic, or xenogenic to the subject. In some embodiments, the subject is a human subject.

In some embodiments, the guide oligonucleotide comprises a nucleic acid sequence complementary to an MECP2 mRNA sequence comprising the SNP associated with Rett syndrome. In other embodiments, the oligonucleotide further comprises one or more adenosine deaminase acting on RNA (ADAR)-recruiting domains.

In some embodiments, the MECP2 polynucleotide encodes a MECP2 protein comprising a pathogenic amino acid comprising a premature stop codon at positions 255, 168, 294 and/or 270, resulting from the SNP.

In some embodiments, the adenosine to inosine alteration substitutes the pathogenic amino acid with a wild type amino acid. In some embodiments, the wild type amino acid at positions 255, 168, 294 and 270 comprises an arginine.

In some embodiments, the guide oligonucleotide comprises the structure:

[Aₘ]-X¹-X²-X³—[Bₙ]

wherein each of A and B is a nucleotide; m and n are each, independently, an integer from 1 to 50; X¹, X², and X³ are each, independently, a nucleotide, wherein at least one of X¹, X², or X³ is an alternative nucleotide.

In other embodiments, the guide oligonucleotide comprises the structure:

[Aₘ]-X¹-X²-X³—[Bₙ]

wherein each of A and B is a nucleotide; m and n are each, independently, an integer from 1 to 50; X¹, X², and X³ are each, independently, a nucleotide, wherein at least one of X¹, X², or X³ has the structure of any one of Formula I-V:

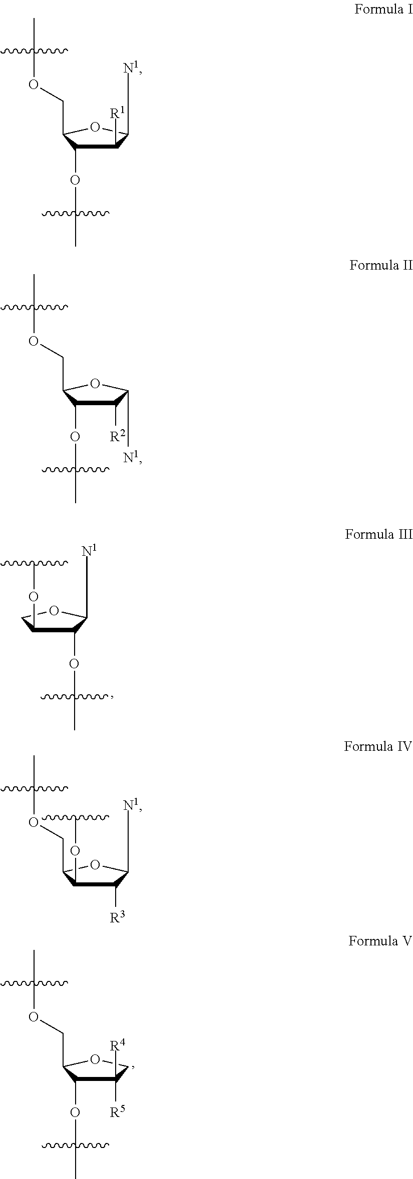

wherein N¹ is hydrogen or a nucleobase; R¹ is hydroxy, halogen, or C₁-C₆ alkoxy; R² is hydrogen, hydroxy, halogen, or C₁-C₆ alkoxy; R³ is hydrogen, hydroxy, halogen, or C₁-C₆ alkoxy; R⁴ is hydrogen, hydroxy, halogen, or C₁-C₆ alkoxy; and R⁵ is hydrogen, hydroxy, halogen, or C₁-C₆ alkoxy. In some embodiments, R⁴ is hydrogen and R⁵ is not hydrogen or hydroxy, R⁵ is hydrogen and R⁴ is not hydrogen, or R⁵ is hydroxy and R⁴ is not hydrogen.

In some embodiments, at least 80% of the nucleotides of [Aₘ] and/or [Bₙ] include a nucleobase, a sugar, and an internucleoside linkage.

In some embodiments, R¹ is hydroxy, halogen, or OCH₃. In other embodiments, R² is hydrogen.

In some embodiments, at least one of X¹, X², or X³ has the structure of Formula I, Formula II, or Formula V; and none of X¹, X², or X³ has the structure of Formula IV or Formula III. In other embodiments, at least one of X¹, X², or X³ has the structure of Formula I or Formula II; and none of X¹, X², or X³ has the structure of Formula III, Formula IV, or Formula V.

In some embodiments, the halogen is fluoro.

In other embodiments, at least one of X¹, X², and X³ has the structure of Formula I, wherein R¹ is fluoro and N¹ is a nucleobase. In some embodiments, X¹ has the structure of Formula I, wherein R¹ is fluoro and N¹ is a nucleobase. In other embodiments, X² has the structure of Formula I, wherein R¹ is fluoro and N¹ is a nucleobase. In some embodiments, X³ has the structure of Formula I, wherein R¹ is fluoro and N¹ is a nucleobase. In other embodiments, at least one of X¹, X², and X³ has the structure of Formula I, wherein R¹ is hydroxy and N¹ is a nucleobase. In some embodiments, X¹ has the structure of Formula I, wherein R¹ is hydroxy and N¹ is a nucleobase. In other embodiments, X² has the structure of Formula I, wherein R¹ is hydroxy and N¹ is a nucleobase. In some embodiments, X³ has the structure of Formula I, wherein R¹ is hydroxy and N¹ is a nucleobase. In other embodiments, at least one of X¹, X², and X³ has the structure of Formula I, wherein R¹ is methoxy and N¹ is a nucleobase. In some embodiments, X¹ has the structure of Formula I, wherein R¹ is methoxy and N¹ is a nucleobase; and each of X² and X³ is a ribonucleotide. In other embodiments, X² has the structure of Formula I, wherein R¹ is methoxy and N¹ is a nucleobase. In some embodiments, X³ has the structure of Formula I, wherein R¹ is methoxy and N¹ is a nucleobase.

In some embodiments, at least one of X¹, X², and X³ has the structure of Formula II, wherein R² is hydrogen and N¹ is a nucleobase. In some embodiments, X² has the structure of Formula II, wherein R² is hydrogen and N¹ is a nucleobase.

In other embodiments, at least one of X¹ and X² has the structure of Formula V. In some embodiments, X² has the structure of Formula V, wherein R⁴ is hydrogen and R⁵ is hydrogen. In other embodiments, X² has the structure of Formula V, wherein R⁴ is hydrogen and R⁵ is hydroxy. In some embodiments, X¹ has the structure of Formula V, wherein R⁴ is hydrogen and R⁵ is hydrogen. In other embodiments, X¹ has the structure of Formula V, wherein R⁴ is hydrogen and R⁵ is hydroxy. In some embodiments, X² has the structure of Formula V, wherein R⁴ is hydrogen and R⁵ is methoxy.

In some embodiments, when X¹ has the structure of any one of Formulas I to V, each of X² and X³ is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; when X² has the structure of any one of Formulas I to V, each of X¹ and X³ is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; when X³ has the structure of any one of Formulas I to V, each of X¹ and X² is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; when X¹ and X² each have the structure of any one of Formulas I to V, X³ is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; when X¹ and X³ each have the structure of any one of Formulas I to V, X² is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; and when X² and X³ each have the structure of any one of Formulas I to V, X¹ is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide.

In other embodiments, when X¹ has the structure of any one of Formulas I to V, each of X² and X³ is, independently, a ribonucleotide, a 2′-F-nucleotide, 2′-O-methoxyethyl-nucleotide, or a DNA-nucleotide; when X² has the structure of any one of Formulas I to V, each of X¹ and X³ is, independently, a ribonucleotide, a 2′-F-nucleotide, 2′-O-methoxyethyl-nucleotide, or a DNA-nucleotide; when X³ has the structure of any one of Formulas I to V, each of X¹ and X² is, independently, a ribonucleotide, a 2′-F-nucleotide, 2′-O-methoxyethyl-nucleotide, or a DNA-nucleotide; when X¹ and X² each have the structure of any one of Formulas I to V, X³ is a ribonucleotide, a 2′-F-nucleotide, 2′-O-methoxyethyl-nucleotide, or a DNA-nucleotide; when X¹ and X³ each have the structure of any one of Formulas I to V, X² is a ribonucleotide, a 2′-F-nucleotide, 2′-O-methoxyethyl-nucleotide, or a DNA-nucleotide; and when X² and X³ each have the structure of any one of Formulas I to V, X¹ is a ribonucleotide, a 2′-F-nucleotide, 2′-O-methoxyethyl-nucleotide, or a DNA-nucleotide.

In some embodiments, when X¹ has the structure of any one of Formulas I to V, each of X² and X³ is a ribonucleotide; when X² has the structure of any one of Formulas I to V, each of X¹ and X³ is a ribonucleotide; when X³ has the structure of any one of Formulas I to V, each of X¹ and X² is a ribonucleotide; when X¹ and X² each have the structure of any one of Formulas I to V, X³ is a ribonucleotide; when X¹ and X³ each have the structure of any one of Formulas I to V, X² is a ribonucleotide; and when X² and X³ each have the structure of any one of Formulas I to V, X¹ is a ribonucleotide.

In some embodiments, none of X¹, X², and X³ has the structure of Formula II, wherein N¹ is a nucleobase. In other embodiments, none of X¹, X², and X³ has the structure of Formula II, wherein N¹ is a cytosine nucleobase.

In some embodiments, X¹ comprises a uracil or thymine nucleobase. In other embodiments, X¹ comprises a uracil nucleobase. In some embodiments, X¹ comprises a hypoxanthine nucleobase. In other embodiments, X¹ comprises a cytosine nucleobase.

In some embodiments, X³ comprises a guanine nucleobase. In other embodiments, X³ comprises a hypoxanthine nucleobase. In some embodiments, X³ comprises an adenine nucleobase.

In some embodiments, X² comprises a cytosine or 5-methylcytosine nucleobase. In other embodiments, X² comprises a cytosine nucleobase. In some embodiments, X² has the structure of any one of Formula I-V. In other embodiments, X² is not a 2′-O-methyl-nucleotide.

In some embodiments, X¹, X², and X³ are not 2′-O-methyl-nucleotides.

In some embodiments, the guide oligonucleotide comprises the structure:

[Aₘ]-X¹-X²-X³—[Bₙ]

wherein each of A and B is a nucleotide; m and n are each, independently, an integer from 1 to 50; X¹, X², and X³ are each, independently, a nucleotide, wherein at least one of X¹, X², or X³ has the structure of any one of Formula VI-XI:

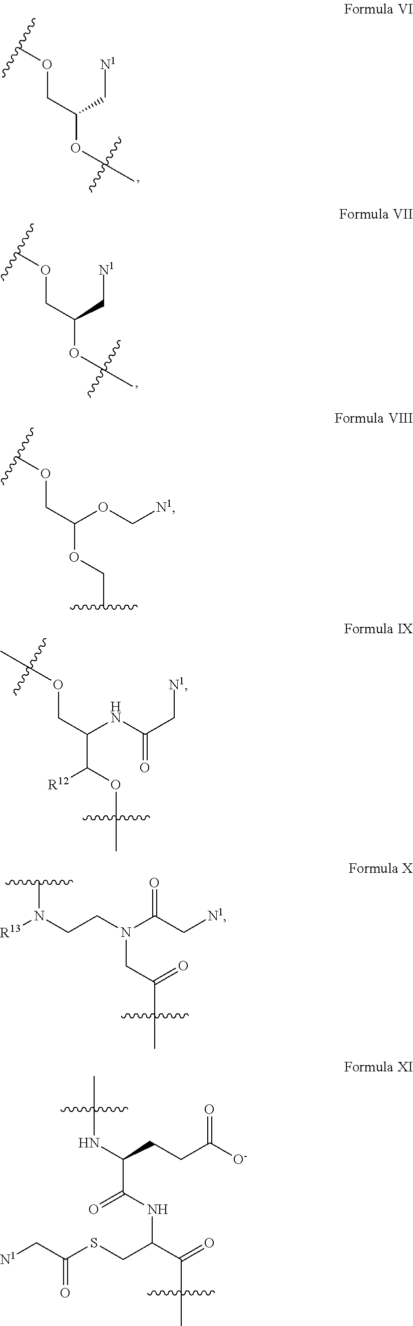

wherein N¹ is hydrogen or a nucleobase; R¹² is hydrogen, hydroxy, fluoro, halogen, C₁-C₆ alkyl, C₁-C₆ heteroalkyl, or C₁-C₆ alkoxy; R¹³ is hydrogen or C₁-C₆ alkyl, wherein at least one of X¹, X², or X³ has the structure of any one of Formula VI-IX.

In some embodiments, at least 80% of the nucleotides of [Aₘ] and/or [Bₙ] include a nucleobase, a sugar, and an internucleoside linkage.

In some embodiments, R¹² is hydrogen, halogen, C₁-C₆ alkyl, or C₁-C₆ heteroalkyl. In other embodiments, the halogen is fluoro. In some embodiments, R¹² is hydrogen or C₁-C₆ alkyl; In other embodiments, R¹² is hydrogen.

In some embodiments, at least one of X¹, X², and X³ has the structure of Formula VI, and N¹ is a nucleobase. In other embodiments, X¹ has the structure of Formula VI, and NV is a nucleobase. In some embodiments, X² has the structure of Formula VI, and N¹ is a nucleobase.

In some embodiments, at least one of X¹, X², and X³ has the structure of Formula VII, and N¹ is a nucleobase. In some embodiments, X¹ has the structure of Formula VII, and N¹ is a nucleobase. In other embodiments, X² has the structure of Formula VII, and N¹ is a nucleobase.

In some embodiments, at least one of X¹, X², and X³ has the structure of Formula IX, and N¹ is a nucleobase. In some embodiments, X¹ has the structure of Formula IX, and N¹ is a nucleobase. In other embodiments, X² has the structure of Formula IX, and N¹ is a nucleobase.

In some embodiments, at least one of X¹, X², and X³ has the structure of Formula VIII, and N¹ is a nucleobase. In some embodiments, X¹ has the structure of Formula VIII, and N¹ is a nucleobase. In other embodiments, X² has the structure of Formula VIII, and N¹ is a nucleobase.

In some embodiments, X² does not have the structure of Formula VI. In other embodiments, X³ does not have the structure of Formula VI. In some embodiments, X² does not have the structure of Formula VII. In other embodiments, X³ does not have the structure of Formula VII. In some embodiments, X² does not have the structure of Formula IX. In other embodiments, X² has the structure of Formula VI or Formula VII.

In some embodiments, when X¹ has the structure of any one of Formulas VI to XI, each of X² and X³ is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; when X² has the structure of any one of Formulas VI to XI, each of X¹ and X³ is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; when X³ has the structure of any one of Formulas VI to XI, each of X¹ and X² is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; when X¹ and X² each have the structure of any one of Formulas VI to XI, X³ is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; when X¹ and X³ each have the structure of any one of Formulas VI to XI, X² is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; and when X² and X³ each have the structure of any one of Formulas VI to XI, X¹ is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide.

In other embodiments, when X¹ has the structure of any one of Formulas VI to XI, each of X² and X³ is, independently, a ribonucleotide, a 2′-F-nucleotide, 2′-O-methoxyethyl-nucleotide, or a DNA-nucleotide; when X² has the structure of any one of Formulas VI to XI, each of X¹ and X³ is, independently, a ribonucleotide, a 2′-F-nucleotide, 2′-O-methoxyethyl-nucleotide, or a DNA-nucleotide; when X³ has the structure of any one of Formulas VI to XI, each of X¹ and X² is, independently, a ribonucleotide, a 2′-F-nucleotide, 2′-O-methoxyethyl-nucleotide, or a DNA-nucleotide; when X¹ and X² each have the structure of any one of Formulas VI to XI, X³ is a ribonucleotide, a 2′-F-nucleotide, 2′-O-methoxyethyl-nucleotide, or a DNA-nucleotide; when X¹ and X³ each have the structure of any one of Formulas VI to XI, X² is a ribonucleotide, a 2′-F-nucleotide, 2′-O-methoxyethyl-nucleotide, or a DNA-nucleotide; and when X² and X³ each have the structure of any one of Formulas VI to XI, X¹ is a ribonucleotide, a 2′-F-nucleotide, 2′-O-methoxyethyl-nucleotide, or a DNA-nucleotide.

In some embodiments, when X¹ has the structure of any one of Formulas VI to XI, each of X² and X³ is a ribonucleotide; when X² has the structure of any one of Formulas VI to XI, each of X¹ and X³ is a ribonucleotide; when X³ has the structure of any one of Formulas VI to XI, each of X¹ and X² is a ribonucleotide; when X¹ and X² each have the structure of any one of Formulas VI to XI, X³ is a ribonucleotide; when X¹ and X³ each have the structure of any one of Formulas VI to XI, X² is a ribonucleotide; and when X² and X³ each have the structure of any one of Formulas VI to XI, X¹ is a ribonucleotide.

In some embodiments, X¹ comprises a hypoxanthine nucleobase. In other embodiments, X¹ comprises a uracil nucleobase. In some embodiments, X¹ comprises a cytosine nucleobase. In other embodiments, X³ comprises a hypoxanthine nucleobase. In some embodiments, X³ comprises a guanine nucleobase. In other embodiments, X³ comprises a adenine nucleobase. In some embodiments, X² comprises a cytosine nucleobase. In other embodiments, X² comprises a uracil nucleobase. In some embodiments, X² does not include a nucleobase. In other embodiments, X² is not a 2′-O-methyl-nucleotide. In some embodiments, X¹, X², and X³ are not 2′-O-methyl-nucleotides.

In some embodiments, the guide oligonucleotide comprises the structure:

[Aₘ]-X¹-X²-X³—[Bₙ]

wherein each of A and B is a nucleotide; m and n are each, independently, an integer from 1 to 50; X¹, X², and X³ are each, independently, a nucleotide, wherein at least one of X¹, X², and X³ has the structure of any one of Formula XII-XV:

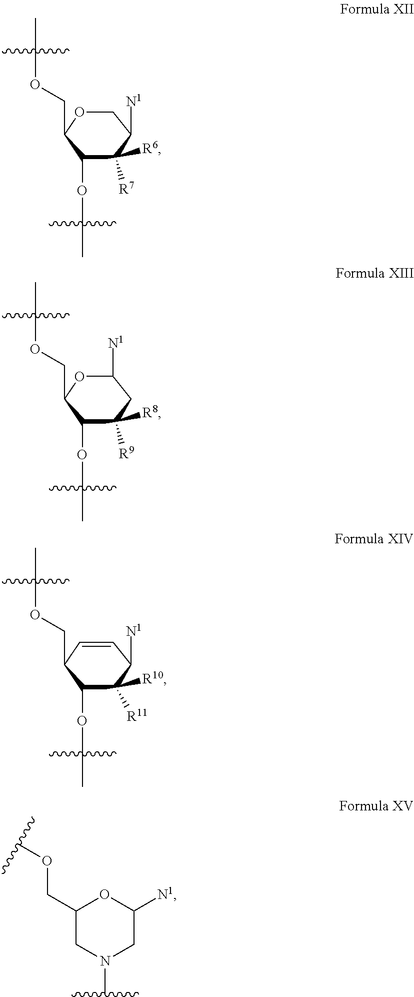

wherein N¹ is hydrogen or a nucleobase; R⁶ is hydrogen, hydroxy, or halogen; R⁷ is hydrogen, hydroxy, halogen, or C₁-C₆ alkoxy; R⁸ is hydrogen or halogen; R⁹ is hydrogen or hydroxy, halogen, or C₁-C₆ alkoxy; R¹⁰ Is hydrogen or halogen; and R¹¹ is hydrogen or hydroxy, halogen, or C₁-C₆ alkoxy.

In some embodiments, at least 80% of the nucleotides of [Aₘ] and/or [Bₙ] include a nucleobase, a sugar, and an internucleoside linkage.

In some embodiments, halogen is fluoro.

In some embodiments, C₁-C₆ alkoxy is OCH₃.

In some embodiments, at least one of X¹, X², and X³ has the structure of Formula XIII, in which each of R⁸ and R⁹ is hydrogen. In some embodiments, X¹ has the structure of Formula XIII, in which each of R⁸ and R⁹ is hydrogen. In other embodiments, X² has the structure of Formula XIII, in which each of R⁸ and R⁹ is hydrogen. In some embodiments, X² has the structure of any one of Formula XII-XV.

In some embodiments, when X¹ has the structure of any one of Formulas XII-XV, each of X² and X³ is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; when X² has the structure of any one of Formulas XII-XV, each of X¹ and X³ is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; when X³ has the structure of any one of Formulas XII-XV, each of X¹ and X² is, independently, a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; when X¹ and X² each have the structure of any one of Formulas XII-XV, X³ is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; when X¹ and X³ each have the structure of any one of Formulas XII-XV, X² is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide; and when X² and X³ each have the structure of any one of Formulas XII-XV, X¹ is a ribonucleotide, a 2′-O—C₁-C₆ alkyl-nucleotide, a 2′-amino-nucleotide, an arabinonucleic acid-nucleotide, a bicyclic-nucleotide, a 2′-F-nucleotide, 2′-O-methoxyethyl-nucleotide, a constrained ethyl-nucleotide, a LNA-nucleotide, or a DNA-nucleotide.

In other embodiments, when X¹ has the structure of any one of Formulas XII-XV, each of X² and X³ is, independently, a ribonucleotide, a 2′-F-nucleotide, 2′-O-methoxyethyl-nucleotide, or a DNA-nucleotide; when X² has the structure of any one of Formulas XII-XV, each of X¹ and X³ is, independently, a ribonucleotide, a 2′-F-nucleotide, 2′-O-methoxyethyl-nucleotide, or a DNA-nucleotide; when X³ has the structure of any one of Formulas XII-XV, each of X¹ and X² is, independently, a ribonucleotide, a 2′-F-nucleotide, 2′-O-methoxyethyl-nucleotide, or a DNA-nucleotide; when X¹ and X² each have the structure of any one of Formulas XII-XV, X³ is a ribonucleotide, a 2′-F-nucleotide, 2′-O-methoxyethyl-nucleotide, or a DNA-nucleotide; when X¹ and X³ each have the structure of any one of Formulas XII-XV, X² is a ribonucleotide, a 2′-F-nucleotide, 2′-O-methoxyethyl-nucleotide, or a DNA-nucleotide; and when X² and X³ each have the structure of any one of Formulas XII-XV, X¹ is a ribonucleotide, a 2′-F-nucleotide, 2′-O-methoxyethyl-nucleotide, or a DNA-nucleotide.

In some embodiments, when X¹ has the structure of any one of Formulas XII-XV, each of X² and X³ is a ribonucleotide; when X² has the structure of any one of Formulas XII-XV, each of X¹ and X³ is a ribonucleotide; when X³ has the structure of any one of Formulas XII-XV, each of X¹ and X² is a ribonucleotide; when X¹ and X² each have the structure of any one of Formulas XII-XV, X³ is a ribonucleotide; when X¹ and X³ each have the structure of any one of Formulas XII-XV, X² is a ribonucleotide; and when X² and X³ each have the structure of any one of Formulas XII-XV, X¹ is a ribonucleotide.

In some embodiments, X¹ includes a hypoxanthine nucleobase. In other embodiments, X¹ includes a uracil nucleobase. In some embodiments, X¹ includes a cytosine nucleobase. In other embodiments, X³ includes a hypoxanthine nucleobase. In some embodiments, X³ includes an adenine nucleobase. In other embodiments, X² includes a cytosine nucleobase. In some embodiments, X² includes a uracil nucleobase. In other embodiments, X² does not include a nucleobase. In some embodiments, X² is not a 2′-O-methyl-nucleotide. In other embodiments, X¹, X², and X³ are not 2′-O-methyl-nucleotides.

In some embodiments, [Aₘ] comprises at least one nuclease resistant nucleotide. In other embodiments, [Aₘ] comprises at least one 2′-O—C₁-C₆ alkyl-nucleotide, at least one 2′-amino-nucleotide, at least one arabino nucleic acid-nucleotide, at least one bicyclic-nucleotide, at least one 2′-F-nucleotide, at least one 2′-O-methoxyethyl-nucleotide, at least one constrained ethyl (cEt)-nucleotide, at least one LNA-nucleotide, and/or at least one DNA-nucleotide.

In some embodiments, [Aₘ] comprises at least one 2′-O-methyl-nucleotide, at least one 2′-F-nucleotide, at least one 2′-O-methoxyethyl-nucleotide, at least one cEt-nucleotide, at least one LNA-nucleotide, and/or at least one DNA-nucleotide. In other embodiments, [Aₘ] comprises at least five terminal 2′-O-methyl-nucleotides. In some embodiments, [Aₘ] comprises at least one phosphorothioate linkage. In other embodiments, [Aₘ] comprises at least four terminal phosphorothioate linkages. In some embodiments, at least one phosphorothioate linkage is stereopure.

In some embodiments, [Bₙ] comprises at least one nuclease resistant nucleotide. In other embodiments, [Bₙ] comprises at least one at least one 2′-O—C₁-C₆ alkyl-nucleotide, at least one 2′-amino-nucleotide, at least one arabino nucleic acid-nucleotide, at least one bicyclic-nucleotide, at least one 2′-F-nucleotide, at least one 2′-O-methoxyethyl-nucleotide, at least one cEt-nucleotide, at least one LNA-nucleotide, and/or at least one DNA-nucleotide.

In some embodiments, [Bₙ] comprises at least one 2′-O-methyl-nucleotide, at least one 2′-F-nucleotide, at least one 2′-O-methoxyethyl-nucleotide, at least one cEt-nucleotide, at least one LNA-nucleotide, and/or at least one DNA-nucleotide. In other embodiments, [Bₙ] comprises at least five terminal 2′-O-methyl-nucleotides. In some embodiments, [Bₙ] comprises at least one phosphorothioate linkage. In other embodiments, [B₁] comprises at least four terminal phosphorothioate linkages. In some embodiments, at least one phosphorothioate linkage is stereopure.

In some embodiments, at least 20% of the nucleotides of [Aₘ] and [Bₙ] combined are 2′-O-methyl-nucleotides.

In some embodiments, the oligonucleotide further comprises a 5′-cap structure. In other embodiments, the oligonucleotide comprises at least one alternative nucleobase. In some embodiments, the 5′-terminal nucleotide is a 2′-amino-nucleotide.

In other embodiments, A and B combined consist of 18 to 80 nucleotides. In some embodiments, m is 5 to 40. In other embodiments, n is 5 to 40.

In some embodiments, m and n are each, independently, an integer from 5 to 40; at least one of X¹, X², and X³ has the structure of Formula I, wherein R¹ is fluoro, hydroxy, or methoxy and N¹ is a nucleobase, or the structure of Formula V, wherein R⁴ is hydrogen and R⁵ is hydrogen; each of X¹, X², and X³ that does not have the structure of Formula I or Formula V is a ribonucleotide; [Aₘ] and [Bₙ] each comprise at least five terminal 2′-O-methyl-nucleotides and at least four terminal phosphorothioate linkages; and at least 20% of the nucleotides of [Aₘ] and [Bₙ] combined are 2′-O-methyl-nucleotides.

In other embodiments, m and n are each, independently, an integer from 5 to 40; at least one of X¹, X², and X³ has the structure of Formula VI, Formula VII, Formula VIII, or Formula IX, wherein N¹ is a nucleobase and each of X¹, X², and X³ that does not have the structure of Formula VI, Formula VII, Formula VIII, or Formula IX is a ribonucleotide; [Aₘ] and [Bₙ] each include at least five terminal 2′-O-methyl-nucleotides and at least four terminal phosphorothioate linkages; and at least 20% of the nucleotides of [Aₘ] and [Bₙ] combined are 2′-O-methyl-nucleotides.

In some embodiments, m and n are each, independently, an integer from 5 to 40; at least of X¹, X², and X³ has the structure of Formula XIII, wherein R⁸ and R⁹ are each hydrogen, and each of X¹, X² and X³ that does not have the structure of Formula XII is a ribonucleotide; [Aₘ] and [Bₙ] each include at least five terminal 2′-O-methyl-nucleotides and at least four terminal phosphorothioate linkages; and at least 20% of the nucleotides of [Aₘ] and [B₁] combined are 2′-O-methyl-nucleotides.

### DETAILED DESCRIPTION OF THE INVENTION

The present invention provides methods of editing a MECP2 polynucleotide, e.g., a MECP2 polynucleotide comprising a single nucleotide polymorphism (SNP) associated with Rett syndrome, and methods for treating or preventing a MECP2-associated disease, e.g., Rett syndrome, in a subject using a guide oligonucleotide capable of effecting an adenosine deaminase acting on RNA (ADAR)-mediated adenosine to inosine alteration in the target gene, e.g., an ADAR-mediated adenosine to inosine alternation of the SNP associated with Rett syndrome.

The present invention provides methods for the site specific editing of MECP2 in a cell, without the need to transduce or transfect the cell with genetically engineered editing enzymes. The design of the guide oligonucleotides of the present invention allows the recruitment of the ADAR enzyme, to the specific editing sites disclosed herein. The methods of the present invention can conveniently be used to make changes in MECP2, for example to reverse mutations that are involved in, or cause, MECP2-associated disease, thereby alleviating the symptoms of the disease. This is of great advantage when used in treating the MECP2-associated disease, e.g., Rett syndrome. Further, the guide oligonucleotides used in the methods of the present invention provide an ease of delivery and avoid any immune response, e.g., associated with viral vectors. In addition, editing of the MECP2 mRNA preserves the endogenous expression level, transcriptional control, and splice variants of the wild type gene. This is particularly important for the MECP2 gene because over and under expression of the gene can both cause a deleterious phenotype.

The following detailed description discloses methods for editing a MECP2 polynucleotide using a guide oligonucleotide capable of effecting an ADAR-mediated adenosine to inosine alteration, how to make and use compositions containing the guide oligonucleotides capable of effecting an ADAR-mediated adenosine to inosine alteration, as well as compositions, uses, and methods for treating subjects having a MECP2-associated disease that would benefit from editing the sequence of a MECP2 gene.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including, but not limited to”.

The term “or” is used herein to mean, and is used interchangeably with, the term “and/or,” unless context clearly indicates otherwise.

The term “about” is used herein to mean within the typical ranges of tolerances in the art, e.g., acceptable variation in time between doses, acceptable variation in dosage unit amount. For example, “about” can be understood as within about 2 standard deviations from the mean. In certain embodiments, about means+10%. In certain embodiments, about means+5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range.

As used herein, “central triplet” or the “triplet” is understood as the three nucleotides opposite the target adenosine in the target RNA, wherein the middle nucleotide in the central triplet is directly opposite the target adenosine. The central triplet does not have to be in the middle (in the center) of the guide oligonucleotide, it may be located more to the 3′ as well as to the 5′ end of the guide oligonucleotide, whatever is preferred for a certain target. Central in this aspect has therefore more the meaning of the triplet that is in the center of catalytic activity when it comes to chemical modifications and targeting the target adenosine. It should also be noted that the guide oligonucleotides are sometimes depicted from 3′ to 5′, especially when the target sequence is shown from 5′ to 3′. However, whenever herein the order of nucleotides within the guide oligonucleotide is discussed it is always from 5′ to 3′ of the guide oligonucleotide. The position can also be expressed in terms of a particular nucleotide within the guide oligonucleotide while still adhering to the 5′ to 3′ directionality, in which case other nucleotides 5′ of the said nucleotide are marked as negative positions and those 3′ of it as positive positions. For example, the C in the Central triplet is the nucleotide (at the 0 position) opposite the targeted adenosine and the U would in this case be the −1 nucleotide and the G would then be the +1 nucleotide, etc.

As used herein, “no more than” or “less than” is understood as the value adjacent to the phrase and logical lower values or integers, as logical from context, to zero. For example, an oligonucleotide with “no more than 5 unmodified nucleotides” has 5, 4, 3, 2, 1, or 0 unmodified nucleotides. When “no more than” is present before a series of numbers or a range, it is understood that “no more than” can modify each of the numbers in the series or range.

As used herein, a “MECP2” refers to the well-known gene and protein. MECP2 is also known as Methyl-CpG Binding Protein 2, AUTSX3, MRXS13, MRXSL, MRX16, MRX79, MeCp2, PPMX, RTS, RTT, or RS. MECP2 is an abundant chromosome-binding protein that selectively binds 5-methyl cytosine residues in symmetrically positioned CpG dinucleotides in mammalian genomes (Lewis et al., Cell. 1992 Jun. 12; 69(6):905-14). These residues are preferentially located in the promoter regions of genes that are subject to transcriptional silencing after DNA methylation. MECP2 is the molecular link between DNA methylation and transcriptional silencing by histone deacetylation (Nan et al., Nature. 1998 May 28; 393(6683):386-9.). It contains at least two functional domains: an 85 amino acid methyl-CpG-binding domain, essential for its binding to 5-methyl cytosine (Nan et al., Nucleic Acids Res. 1993 Oct. 25; 21(21):4886-92), and a 104 amino acid transcriptional repression domain that interacts with histone deacetylase and the transcriptional corepressor Sin3A. Interactions between this transcription repressor complex and chromatin-bound MECP2 leads to deacetylation of core histones, which in turn leads to transcriptional repression (Nan et al., 1998). Many of the genes that are known to be regulated by the MECP2 protein play a role in normal brain function, particularly the maintenance of synapses. Reduced expression of MECP2 in Mecp2+/− neural stem cells causes an increase in senescence, impairment of proliferative capacity and accumulation of unrepaired DNA damages (Alessio et al., 2018. Exp. Mol. Med. 50 (3): 1). MECP2 gene expression is tightly regulated, and MECP2 duplication leads to a severe neurodevelopmental phenotype. The role of MECP2 in disease is primarily associated with either a loss of function (under expression) of the MECP2 gene in Rett syndrome or a gain of function in MECP2 duplication syndrome. Many mutations associated with loss of expression of the MECP2 gene have been identified in Rett syndrome patients. In particular, high frequency missense mutations at position 255 of the MECP2 protein is associated with Rett syndrome. The sequence of a human MECP2 mRNA transcript can be found at National Center for Biotechnology Information (NCBI) RefSeq accession number NM_004992 (SEQ ID NO: 57). Additional examples of MECP2 mRNA sequences are readily available using publicly available databases, e.g., GenBank, UniProt, and OMIM.

A “MECP2-associated disease,” as used herein, is intended to include any disease associated with the MECP2 gene or protein. Such a disease may be caused, for example, by MECP2 gene mutations, by excess production of the MECP2 protein, by abnormal cleavage of the MECP2 protein, instability of MECP2, by abnormal interactions between MECP2 and other proteins or other endogenous or exogenous substances. In some embodiments, the “MECP2-associated disease” is the Rett syndrome. Rett syndrome is a severe neurodevelopmental disorder caused by loss of function mutations in MECP2, and is characterized by significant developmental impairment in females. Rett syndrome is an X-linked disease, and the affected females are mosaic with 50% of cells expressing a non-functional copy of MECP2 due to X-inactivation. It is characterized by normal early growth and development followed by a slowing of development, loss of purposeful use of the hands, distinctive hand movements, slowed brain and head growth, problems with walking, seizures, and intellectual disability. The course of Rett syndrome, including the age of onset and the severity of symptoms, varies from child to child. Before the symptoms begin, however, the child generally appears to grow and develop normally, although there are often subtle abnormalities even in early infancy, such as loss of muscle tone (hypotonia), difficulty feeding, and jerkiness in limb movements. Then, gradually, mental and physical symptoms appear. As the syndrome progresses, the child loses purposeful use of her hands and the ability to speak. Other early symptoms may include problems crawling or walking and diminished eye contact. The loss of functional use of the hands is followed by compulsive hand movements such as wringing and washing. The onset of this period of regression is sometimes sudden. Apraxia—the inability to perform motor functions—is among the most severely disabling feature of Rett syndrome, interfering with every body movement, including eye gaze and speech. Children with Rett syndrome often exhibit autistic-like behaviors in the early stages. Other symptoms may include walking on the toes, sleep problems, a wide-based gait, teeth grinding and difficulty chewing, slowed growth, seizures, cognitive disabilities, and breathing difficulties while awake such as hyperventilation, apnea (breath holding), and air swallowing (“Rett Syndrome Fact Sheet”. National Institute of Neurological Disorders and Stroke. October 2017). There are generally four stages of Rett syndrome. Stage I, called early onset, typically begins between 6 and 18 months of age. This stage is often overlooked because symptoms of the disorder may be vague, and parents and doctors may not notice the subtle slowing of development at first. This stage usually lasts for a few months but can continue for more than a year. Stage II, or the rapid destructive stage, usually begins between ages 1 and 4 and may last for weeks or months. Its onset may be rapid or gradual as the child loses purposeful hand skills and spoken language. Characteristic hand movements such as wringing, washing, clapping, or tapping, as well as repeatedly moving the hands to the mouth often begin during this stage. Breathing irregularities such as episodes of apnea and hyperventilation may occur, although breathing usually improves during sleep. Some girls also display autistic-like symptoms such as loss of social interaction and communication. Walking may be unsteady and initiating motor movements can be difficult. Slowed head growth is usually noticed during this stage. Stage III, or the plateau or pseudo-stationary stage, usually begins between ages 2 and 10 and can last for years. Apraxia, motor problems, and seizures are prominent during this stage. Stage IV, or the late motor deterioration stage, can last for years or decades. Prominent features include reduced mobility, curvature of the spine (scoliosis) and muscle weakness, rigidity, spasticity, and increased muscle tone with abnormal posturing of an arm, leg, or top part of the body. Repetitive hand movements may decrease and eye gaze usually improves. There are no existing therapies for Rett syndrome.

As used herein, the term “single nucleotide polymorphisms (SNP),” refers to a variation at a single position in a DNA sequence among individuals. If more than 1% of a population does not carry the same nucleotide at a specific position in the DNA sequence, then this variation can be classified as a SNP. If a SNP occurs within a gene, then the gene is described as having more than one allele. In these cases, SNPs may lead to variations in the amino acid sequence. For example, at a specific base position in the human genome, the C nucleotide can appear in most individuals, but in a minority of individuals, the position is occupied by an A. This means that there is a SNP at this specific position, and the two possible nucleotide variations, C or A, are the two alleles for this position.

SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant is a variation in a single nucleotide without any limitations of frequency and can arise in somatic cells. A somatic single nucleotide variation can also be called a single-nucleotide alteration.

Although a particular SNP may not cause a disorder, some SNPs are associated with certain diseases. These associations allow for the use of specific SNPs to evaluate an individual's genetic predisposition to develop a disease. In addition, if certain SNPs are known to be associated with a trait, then examination of certain stretches of DNA near these SNPs will help identify the gene or genes responsible for the trait.

As used herein, the phrase “SNP associated with Rett syndrome” refers to any SNPs that are associated with the onset or development of Rett syndrome. Mutations occur de novo in most Rett syndrome patients, and approximately 40% of patients have 1 of 4 high frequency R>X nonsense mutations. Exemplary SNPs associated with Rett syndrome may include, but are not limited to, any single nucleotide changes in the MECP2 polynucleotide resulting in a premature stop codon at positions 255, 168, 294 and/or 270 of the MECP2 protein. In some embodiments, the SNP associated with Rett syndrome is rs61749721, a SNP commonly referred to as the R255X variant (or mutation) based on the potential change from arginine (encoded by wild type allele) to a premature stop codon (encoded by the rs61749721 allele) at position 255 of the MECP2 protein. In some embodiments, the SNP associated with Rett syndrome is rs61748421, a SNP commonly referred to as the R168X variant (or mutation) based on the potential change from arginine (encoded by rs61748421(C) allele) to a premature stop codon (encoded by the rs61748421(T) allele) at position 168 of the MECP2 protein. In some embodiments, the SNP associated with Rett syndrome is rs61751362, a SNP commonly referred to as the R294X variant (or mutation) based on the potential change from arginine (encoded by rs61751362(C) allele) to a premature stop codon (encoded by the rs61751362(T) allele) at position 294 of the MECP2 protein. In some embodiments, the SNP associated with Rett syndrome is rs61750240, a SNP commonly referred to as the R270X variant (or mutation) based on the potential change from arginine (encoded by rs61750240(C) allele) to a premature stop codon (encoded by the rs61750240(T) allele) at position 270 of the MECP2 protein.

The term “pathogenic amino acid” refers to any amino acid that is not a wild-type amino acid in a protein and which leads to a pathogenesis.

The terms “pathogenic mutation”, “pathogenic variant”, “disease causing mutation”, “disease causing variant”, or “deleterious mutation”, refers to a genetic alteration or mutation that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation comprises a missense mutation. In some embodiments, the pathogenic mutation comprises a splice site mutation, e.g., a splice donor variant, or a splice acceptor variant. In some embodiments, the pathogenic mutation comprises a nonsense mutation. In some embodiments, the pathogenic mutation comprises at least one wild-type allele substituted by at least one pathogenic allele in the target gene.

As used herein, a “premature stop codon” refers to the appearance of a stop codon where there should be a codon corresponding to an amino acid.

The term “adenosine deaminase”, as used herein, refers to a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). In some embodiments, the adenosine deaminase catalyzes the hydrolytic deamination of adenine or adenosine in ribonucleic acid (RNA). The adenosine deaminases may be from any organism, such as a human, chimpanzee, gorilla, monkey, cow, dog, rat, or mouse. In some embodiments, the adenosine deaminase is from a bacterium, such as E. coli, S. aureus, S. typhi, S. putrefaciens, H. influenzae, or C. crescentus. In some embodiments, the deaminase or deaminase domain is a variant of a naturally occurring deaminase from an organism, such as a human, chimpanzee, gorilla, monkey, cow, dog, rat, or mouse. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to a naturally occurring deaminase. For example, deaminase domains are described in International PCT Application Nos. PCT/2017/045381 (WO 2018/027078) and PCT/US2016/058344 (WO 2017/070632), each of which is incorporated herein by reference for its entirety. Also see Komor, A. C., et al., Nature 533, 420-424 (2016); Gaudelli, N. M., et al., Nature 551, 464-471 (2017); Komor, A. C., et al., Science Advances 3:eaao4774 (2017), and Rees, H. A., et al., Nat Rev Genet. 2018; 19(12):770-788. doi: 10.1038/s41576-018-0059-1, the entire contents of which are hereby incorporated by reference.

As used herein, the term “Adenosine deaminases acting on RNA (ADAR)” refers to editing enzymes which can recognize certain structural motifs of double-stranded RNA (dsRNA), bind to dsRNA and convert adenosine to inosine through deamination, resulting in recoding of amino acid codons that may lead to changes to the encoded protein and its function. The nucleobases surrounding the editing site, especially the one immediately 5′ of the editing site and one immediately 3′ to the editing site, which together with the editing site are termed the triplet, play an important role in the deamination of adenosine. A preference for U at the 5′ position and G at the 3′ position relative to the editing site, was revealed from the analysis of yeast RNAs efficiently edited by overexpressed human ADAR2 and ADAR1. (See Wang et al., (2018) Biochemistry, 57: 1640-1651; Eifler et al., (2013) Biochemistry, 52: 7857-7869, and Eggington et al., (2011) Nat. Commun., 319: 1-9.) There are three known ADAR proteins expressed in humans, ADAR1, ADAR2, and ADAR3. ADAR1 and ADAR2 are expressed throughout the body, whereas ADAR3 is expressed only in the brain. ADAR1 and ADAR2 are catalytically active, while ADAR3 is thought to be inactive. Recruiting ADAR to specific sites of selected transcripts and deamination of adenosine regardless of neighboring bases holds great promise for the treatment of disease.

As used herein, the term “ADAR-recruiting domain” refers to nucleotide sequences that may be part of the oligonucleotides of the instant invention and which are able to recruit an ADAR enzyme. For example, such recruiting domains may form stem-loop structures that act as recruitment and binding regions for the ADAR enzyme. Oligonucleotides including such ADAR-recruiting domains may be referred to as “axiomer AONs” or “self-looping AONs.” The ADAR-recruiting domain portion may act to recruit an endogenous ADAR enzyme present in the cell. Such ADAR-recruiting domains do not require conjugated entities or presence of modified recombinant ADAR enzymes. Alternatively, the ADAR-recruiting portion may act to recruit a recombinant ADAR fusion protein that has been delivered to a cell or to a subject via an expression vector construct including a polynucleotide encoding an ADAR fusion protein. Such ADAR-fusion proteins may include the deaminase domain of ADAR1 or ADAR2 enzymes fused to another protein, e.g., to the MS2 bacteriophage coat protein. An ADAR-recruiting domain may be a nucleotide sequence based on a natural substrate (e.g., the GluR2 receptor pre-mRNA; such as a GluR2 ADAR-recruiting domain), a Z-DNA structure, or a domain known to recruit another protein which is part of an ADAR fusion protein, e.g., an MS2 ADAR-recruiting domain known to be recognized by the dsRNA binding regions of ADAR. A stem-loop structure of an ADAR-recruiting domain can be an intermolecular stem-loop structure, formed by two separate nucleic acid strands, or an intramolecular stem loop structure, formed within a single nucleic acid strand.

As used herein, the term “Z-DNA” refers to a left-handed conformation of the DNA double helix or RNA stem loop structures. Such DNA or dsRNA helices wind to the left in a zigzag pattern (as opposed to the right, like the more commonly found B-DNA form). Z-DNA is a known high-affinity ADAR binding substrate and has been shown to bind to human ADAR1 enzyme.

“G,” “C,” “A,” “T,” and “U” each generally stand for a naturally-occurring nucleotide that contains guanine, cytosine, adenine, thymidine, and uracil as a base, respectively. However, it will be understood that the term “nucleotide” can also refer to an alternative nucleotide, as further detailed below, or a surrogate replacement moiety. The skilled person is well aware that guanine, cytosine, adenine, and uracil can be replaced by other moieties without substantially altering the base pairing properties of an oligonucleotide including a nucleotide bearing such replacement moiety. For example, without limitation, a nucleotide including hypoxanthine as its base can base pair with nucleotides containing adenine, cytosine, or uracil. Hence, nucleotides containing uracil, guanine, or adenine can be replaced in the nucleotide sequences of oligonucleotides featured in the invention by a nucleotide containing, for example, hypoxanthine. In another example, adenine and cytosine anywhere in the oligonucleotide can be replaced with guanine and uracil, respectively to form G-U wobble base pairing with the target mRNA. Sequences containing such replacement moieties are suitable for the compositions and methods featured in the invention.

The terms “nucleobase” and “base” include the purine (e.g., adenine and guanine) and pyrimidine (e.g., uracil, thymine, and cytosine) moiety present in nucleosides and nucleotides which form hydrogen bonds in nucleic acid hybridization. In the context of the present invention, the term nucleobase also encompasses alternative nucleobases which may differ from naturally-occurring nucleobases but are functional during nucleic acid hybridization. In this context “nucleobase” refers to both naturally occurring nucleobases such as adenine, guanine, cytosine, thymidine, uracil, xanthine, and hypoxanthine, as well as alternative nucleobases. Such variants are, for example, described in Hirao et al (2012) Accounts of Chemical Research vol 45, page 2055 and Bergstrom (2009) Current Protocols in Nucleic Acid Chemistry Suppl. 37 Chapter 1, unit 4.1.

In a some embodiments the nucleobase moiety is modified by changing the purine or pyrimidine into a modified purine or pyrimidine, such as substituted purine or substituted pyrimidine, such as an “alternative nucleobase” selected from isocytosine, pseudoisocytosine, 5-methylcytosine, 5-thiozolo-cytosine, 5-propynyl-cytosine, 5-propynyl-uracil, 5-bromouracil, 5-thiazolo-uracil, 2-thio-uracil, pseudouracil, 1-methylpseudouracil, 5-methoxyuracil, 2′-thio-thymine, hypoxanthine, diaminopurine, 6-aminopurine, 2-aminopurine, 2,6-diaminopurine, and 2-chloro-6-aminopurine.

The nucleobase moieties may be indicated by the letter code for each corresponding nucleobase, e.g. A, T, G, C, or U, wherein each letter may optionally include alternative nucleobases of equivalent function.

A “sugar” or “sugar moiety,” includes naturally occurring sugars having a furanose ring. A sugar also includes an “alternative sugar,” defined as a structure that is capable of replacing the furanose ring of a nucleoside. In certain embodiments, alternative sugars are non-furanose (or 4′-substituted furanose) rings or ring systems or open systems. Such structures include simple changes relative to the natural furanose ring, such as a six-membered ring, or may be more complicated as is the case with the non-ring system used in peptide nucleic acid. Alternative sugars may also include sugar surrogates wherein the furanose ring has been replaced with another ring system such as, for example, a morpholino or hexitol ring system. Sugar moieties useful in the preparation of oligonucleotides having motifs include, without limitation, β-D-ribose, β-D-2′-deoxyribose, substituted sugars (such as 2′, 5′ and bis substituted sugars), 4′-S-sugars (such as 4′-S-ribose, 4′-S-2′-deoxyribose and 4′-S-2′-substituted ribose), bicyclic alternative sugars (such as the 2′-O—CH₂-4′ or 2′-O—(CH₂)₂-4′ bridged ribose derived bicyclic sugars) and sugar surrogates (such as when the ribose ring has been replaced with a morpholino or a hexitol ring system). The type of heterocyclic base and internucleoside linkage used at each position is variable and is not a factor in determining the motif. In most nucleosides having an alternative sugar moiety, the heterocyclic nucleobase is generally maintained to permit hybridization.

A “nucleotide,” as used herein refers to a monomeric unit of an oligonucleotide or polynucleotide that includes a nucleoside and an internucleoside linkage. The internucleoside linkage may or may not include a phosphate linkage. Similarly, “linked nucleosides” may or may not be linked by phosphate linkages. Many “alternative internucleoside linkages” are known in the art, including, but not limited to, phosphorothioate and boronophosphate linkages. Alternative nucleosides include bicyclic nucleosides (BNAs) (e.g., locked nucleosides (LNAs) and constrained ethyl (cEt) nucleosides), peptide nucleosides (PNAs), phosphotriesters, phosphorothionates, phosphoramidates, and other variants of the phosphate backbone of native nucleoside, including those described herein.

An “alternative nucleotide” as used herein, refers to a nucleotide having an alternative nucleobase or an alternative sugar, and an internucleoside linkage, which may include alternative nucleoside linkages.

The term “nucleoside” refers to a monomeric unit of an oligonucleotide or a polynucleotide having a nucleobase and a sugar moiety. A nucleoside may include those that are naturally-occurring as well as alternative nucleosides, such as those described herein. The nucleobase of a nucleoside may be a naturally-occurring nucleobase or an alternative nucleobase. Similarly, the sugar moiety of a nucleoside may be a naturally-occurring sugar or an alternative sugar.

The term “alternative nucleoside” refers to a nucleoside having an alternative sugar or an alternative nucleobase, such as those described herein.

The term “nuclease resistant nucleotide” as used herein refers to nucleotides which limit nuclease degradation of oligonucleotides. Nuclease resistant nucleotides generally increase stability of oligonucleotides by being poor substrates for the nucleases. Nuclease resistant nucleotides are known in the art, e.g., 2′-O-methyl-nucleotides and 2′-fluoro-nucleotides.

The terms “oligonucleotide” and “polynucleotide” as used herein, are defined as it is generally understood by the skilled person as a molecule including two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. The oligonucleotide of the invention may be man-made, and is chemically synthesized, and is typically purified or isolated. Oligonucleotide is also intended to include (i) compounds that have one or more furanose moieties that are replaced by furanose derivatives or by any structure, cyclic or acyclic, that may be used as a point of covalent attachment for the base moiety, (ii) compounds that have one or more phosphodiester linkages that are either modified, as in the case of phosphoramidate or phosphorothioate linkages, or completely replaced by a suitable linking moiety as in the case of formacetal or riboacetal linkages, and/or (iii) compounds that have one or more linked furanose-phosphodiester linkage moieties replaced by any structure, cyclic or acyclic, that may be used as a point of covalent attachment for the base moiety. The oligonucleotide of the invention may include one or more alternative nucleosides or nucleotides (e.g., including those described herein). It is also understood that oligonucleotide includes compositions lacking a sugar moiety or nucleobase but is still capable of forming a pairing with or hybridizing to a target sequence.

“Oligonucleotide” refers to a short polynucleotide (e.g., of 100 or fewer linked nucleosides).

The phrases “an oligonucleotide that is capable of effecting an adenosine deaminase acting on RNA (ADAR)-mediated adenosine to inosine alteration” or “a guide oligonucleotide that is capable of effecting an ADAR-mediated adenosine to inosine alteration” refer to an oligonucleotide that is specific for a target sequence and is capable to be utilized for the deamination reaction of a specific adenosine in a target sequence through an ADAR-mediated pathway. The oligonucleotide may comprise a nucleic acid sequence complementary to a target sequence, e.g., a MECP2 mRNA sequence comprising the SNP associated with Rett syndrome. In some embodiments, the oligonucleotides may comprise a nucleic acid sequence complementary to target mRNA with the exception of at least one mismatch. The oligonucleotide includes a mismatch opposite the target adenosine. In some embodiments, the oligonucleotides for use in the methods of the present invention do not include those used by any other gene editing technologies known in the art, e.g., CRISPR.

The oligonucleotide may be of any length, and may range from about 10-100 bases in length, e.g., about 15-100 bases in length or about 18-100 bases in length, for example, about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 bases in length, such as about 15-50, 15-49, 15-48, 15-47, 15-46, 15-45, 15-44, 15-43, 15-42, 15-41, 15-40, 15-39, 15-38, 15-37, 15-36, 15-35, 15-34, 15-33, 15-32, 15-31, 15-31, 15-30, 18-50, 18-49, 18-48, 18-47, 18-46, 18-45, 18-44, 18-43, 18-42, 18-41, 18-40, 18-39, 18-38, 18-37, 18-36, 18-35, 18-34, 18-33, 18-32, 18-31, 18-31, 18-30, 19-50, 19-49, 19-48, 19-47, 19-46, 19-45, 19-44, 19-43, 19-42, 19-41, 19-40, 19-39, 19-38, 19-37, 19-36, 19-35, 19-34, 19-33, 19-32, 19-31, 19-31, 19-30, 20-50, 20-49, 20-48, 20-47, 20-46, 20-45, 20-44,20-43, 20-42, 20-41, 20-40, 20-39, 20-38, 20-37, 20-36, 20-35, 20-34, 20-33, 20-32, 20-31, 20-31, 20-30, 21-50, 21-49, 21-48, 21-47, 21-46, 21-45, 21-44, 21-43, 21-42, 21-41, 21-40, 21-39, 21-38, 21-37, 21-36, 21-35, 21-34, 21-33, 21-32, 21-31, 21-31, or 21-30 bases in length. Ranges and lengths intermediate to the above recited ranges and lengths are also contemplated to be part of the invention.

The term “linker” or “linking group” is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety to an oligonucleotide (e.g. the termini of region A or C). In some embodiments of the invention the conjugate or oligonucleotide conjugate of the invention may optionally, include a linker region which is positioned between the oligonucleotide and the conjugate moiety. In some embodiments, the linker between the conjugate and oligonucleotide is biocleavable. Phosphodiester containing biocleavable linkers are described in more detail in WO 2014/076195 (herein incorporated by reference).

“Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. Complementary sequences between an oligonucleotide and a target sequence as described herein, include base-pairing of the oligonucleotide or polynucleotide including a first nucleotide sequence to an oligonucleotide or polynucleotide including a second nucleotide sequence over the entire length of one or both nucleotide sequences. Such sequences can be referred to as “fully complementary” with respect to each other herein. However, where a first sequence is referred to as “substantially complementary” with respect to a second sequence herein, the two sequences can be fully complementary, or they can form one or more, but generally no more than 5, 4, 3 or 2 mismatched base pairs upon hybridization for a duplex up to 30 base pairs, while retaining the ability to hybridize under the conditions most relevant to their ultimate application, e.g., deamination of an adenosine. “Substantially complementary” can also refer to a polynucleotide that is substantially complementary to a contiguous portion of the mRNA of interest (e.g., an mRNA having a target adenosine). For example, a polynucleotide is complementary to at least a part of the mRNA of interest if the sequence is substantially complementary to a non-interrupted portion of the mRNA of interest. In some embodiments, the oligonucleotide, as described herein, is at least 50%, at least 55%, at least 60%, at least 65%, at least 70%, at least 75% at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% complementary to the target sequence.

As used herein, and unless otherwise indicated, the term “complementary,” when used to describe a first nucleotide or nucleoside sequence in relation to a second nucleotide or nucleoside sequence, refers to the ability of an oligonucleotide or polynucleotide including the first nucleotide or nucleoside sequence to hybridize and form a duplex structure under certain conditions with an oligonucleotide or polynucleotide including the second nucleotide sequence, as will be understood by the skilled person. Such conditions can, for example, be stringent conditions, where stringent conditions can include: 400 mM NaCl, 40 mM PIPES pH 6.4, 1 mM EDTA, 50° C., or 70° C., for 12-16 hours followed by washing (see, e.g., “Molecular Cloning: A Laboratory Manual, Sambrook, et al. (1989) Cold Spring Harbor Laboratory Press). Other conditions, such as physiologically relevant conditions as can be encountered inside an organism, can apply. The skilled person will be able to determine the set of conditions most appropriate for a test of complementarity of two sequences in accordance with the ultimate application of the hybridized nucleotides or nucleosides.

As used herein, the terms “variant” and “derivative” are used interchangeably and refer to naturally-occurring, synthetic, and semi-synthetic analogues of a compound, peptide, protein, or other substance described herein. A variant or derivative of a compound, peptide, protein, or other substance described herein may retain or improve upon the biological activity of the original material.

The term“mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4th ed., Cold Spring Harbor Laboratory Press, Cold Spring Harbor, N.Y. (2012)). In some embodiments, the presently disclosed compositions can efficiently generate an“intended mutation”, such as a point mutation, in a nucleic acid (e.g., a nucleic acid within a genome of a subject) without generating a significant number of unintended mutations, such as unintended point mutations. In some embodiments, an intended mutation is a mutation that is generated by a specific guide oligonucleotide, specifically designed to generate the intended mutation. In general, mutations made or identified in a sequence (e.g., an amino acid sequence as described herein) are numbered in relation to a reference (or wild type) sequence, i.e., a sequence that does not contain the mutations. The skilled practitioner in the art would readily understand how to determine the position of mutations in amino acid and nucleic acid sequences relative to a reference sequence.

The term “contacting,” as used herein, includes contacting a target gene, e.g., MECP2 by any means. In some embodiments, a target gene is contacted with a guide oligonucleotide in a cell. Contacting a MECP2 polynucleotide in a cell with a guide oligonucleotide includes contacting the MECP2 polynucleotide in a cell in vitro with the guide oligonucleotide or contacting the MECP2 polynucleotide in a cell in vivo with the guide oligonucleotide.

Contacting a cell in vitro may be done, for example, by incubating the cell with the guide oligonucleotide. Contacting a cell in vivo may be done, for example, by injecting the guide oligonucleotide into or near the tissue where the cell is located, or by injecting the guide oligonucleotide agent into another area, e.g., the bloodstream or the subcutaneous space, such that the agent will subsequently reach the tissue where the cell to be contacted is located. For example, the guide oligonucleotide may contain and/or be coupled to a ligand that directs the oligonucleotide to a site of interest. Combinations of in vitro and in vivo methods of contacting are also possible. For example, a cell may also be contacted in vitro with a guide oligonucleotide and subsequently transplanted into a subject.

In one embodiment, contacting a cell with a guide oligonucleotide includes “introducing” or “delivering the oligonucleotide into the cell” by facilitating or effecting uptake or absorption into the cell. Absorption or uptake of a guide oligonucleotide can occur through unaided diffusive or active cellular processes, or by auxiliary agents or devices. Introducing a guide oligonucleotide into a cell may be in vitro and/or in vivo. For example, for in vivo introduction, oligonucleotides can be injected into a tissue site or administered systemically. In vitro introduction into a cell includes methods known in the art such as electroporation and lipofection. Further approaches are described herein below and/or are known in the art.

As used herein, “lipid nanoparticle” or “LNP” is a vesicle including a lipid layer encapsulating a pharmaceutically active molecule, such as a nucleic acid molecule, e.g., an oligonucleotide. LNP refers to a stable nucleic acid-lipid particle. LNPs typically contain a cationic, ionizable lipid, a non-cationic lipid, and a lipid that prevents aggregation of the particle (e.g., a PEG-lipid conjugate). LNPs are described in, for example, U.S. Pat. Nos. 6,858,225; 6,815,432; 8,158,601; and 8,058,069, the entire contents of which are hereby incorporated herein by reference.

As used herein, the term “liposome” refers to a vesicle composed of amphiphilic lipids arranged in at least one bilayer, e.g., one bilayer or a plurality of bilayers. Liposomes include unilamellar and multilamellar vesicles that have a membrane formed from a lipophilic material and an aqueous interior. The aqueous portion contains the oligonucleotide composition. The lipophilic material isolates the aqueous interior from an aqueous exterior, which typically does not include the oligonucleotide composition, although in some examples, it may. Liposomes also include “sterically stabilized” liposomes, a term which, as used herein, refers to liposomes including one or more specialized lipids that, when incorporated into liposomes, result in enhanced circulation lifetimes relative to liposomes lacking such specialized lipids.

“Micelles” are defined herein as a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

By “level” is meant a level or activity of a protein, or mRNA encoding the protein, as compared to a reference. The reference can be any useful reference, as defined herein. By a “decreased level” or an “increased level” of a protein is meant a decrease or increase in protein level, as compared to a reference (e.g., a decrease or an increase by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 150%, about 200%, about 300%, about 400%, about 500%, or more; a decrease or an increase of more than about 10%, about 15%, about 20%, about 50%, about 75%, about 100%, or about 200%, as compared to a reference; a decrease or an increase by less than about 0.01-fold, about 0.02-fold, about 0.1-fold, about 0.3-fold, about 0.5-fold, about 0.8-fold, or less; or an increase by more than about 1.2-fold, about 1.4-fold, about 1.5-fold, about 1.8-fold, about 2-fold, about 3-fold, about 3.5-fold, about 4.5-fold, about 5-fold, about 10-fold, about 15-fold, about 20-fold, about 30-fold, about 40-fold, about 50-fold, about 100-fold, about 1000-fold, or more). A level of a protein may be expressed in mass/vol (e.g., g/dL, mg/mL, g/mL, ng/mL) or percentage relative to total protein or mRNA in a sample.

The term “pharmaceutical composition,” as used herein, represents a composition containing a compound described herein formulated with a pharmaceutically acceptable excipient, and preferably manufactured or sold with the approval of a governmental regulatory agency as part of a therapeutic regimen for the treatment of disease in a mammal. Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); for intrathecal injection; for intracerebroventricular injections; for intraparenchymal injection; or in any other pharmaceutically acceptable formulation.

A “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

As used herein, the term “pharmaceutically acceptable salt” means any pharmaceutically acceptable salt of the compound of any of the compounds described herein. For example, pharmaceutically acceptable salts of any of the compounds described herein include those that are within the scope of sound medical judgment, suitable for use in contact with the tissues of humans and animals without undue toxicity, irritation, allergic response and are commensurate with a reasonable benefit/risk ratio. Pharmaceutically acceptable salts are well known in the art. For example, pharmaceutically acceptable salts are described in: Berge et al., J. Pharmaceutical Sciences 66:1-19, 1977 and in Pharmaceutical Salts: Properties, Selection, and Use, (Eds. P. H. Stahl and C. G. Wermuth), Wiley-VCH, 2008. The salts can be prepared in situ during the final isolation and purification of the compounds described herein or separately by reacting a free base group with a suitable organic acid.

The compounds described herein may have ionizable groups so as to be capable of preparation as pharmaceutically acceptable salts. These salts may be acid addition salts involving inorganic or organic acids or the salts may, in the case of acidic forms of the compounds described herein, be prepared from inorganic or organic bases. Frequently, the compounds are prepared or used as pharmaceutically acceptable salts prepared as addition products of pharmaceutically acceptable acids or bases. Suitable pharmaceutically acceptable acids and bases and methods for preparation of the appropriate salts are well-known in the art. Salts may be prepared from pharmaceutically acceptable non-toxic acids and bases including inorganic and organic acids and bases. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration. By “reference standard or level” is meant a value or number derived from a reference sample. A “normal control value” is a pre-determined value indicative of non-disease state, e.g., a value expected in a healthy control subject. Typically, a normal control value is expressed as a range (“between X and Y”), a high threshold (“no higher than X”), or a low threshold (“no lower than X”). A subject having a measured value within the normal control value for a particular biomarker is typically referred to as “within normal limits” for that biomarker. A normal reference standard or level can be a value or number derived from a normal subject not having a disease or disorder; a subject that has been treated with a compound described herein. In preferred embodiments, the reference sample, standard, or level is matched to the sample subject sample by at least one of the following criteria: age, weight, sex, disease stage, and overall health. A standard curve of levels of a purified protein, e.g., any described herein, within the normal reference range can also be used as a reference.

As used herein, the term “subject” refers to any organism to which a composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the term “administration” refers to the administration of a composition (e.g., a compound or a preparation that includes a compound as described herein) to a subject or system. Administration to an animal subject (e.g., to a human) may be by any appropriate route, such as the one described herein.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the terms “effective amount,” “therapeutically effective amount,” and “a “sufficient amount” of an agent that results in a therapeutic effect (e.g., in a cell or a subject) described herein refer to a quantity sufficient to, when administered to the subject, including a human, effect beneficial or desired results, including clinical results, and, as such, an “effective amount” or synonym thereto depends on the context in which it is being applied. For example, in the context of treating a disorder, it is an amount of the agent that is sufficient to achieve a treatment response as compared to the response obtained without administration. The amount of a given agent will vary depending upon various factors, such as the given agent, the pharmaceutical formulation, the route of administration, the type of disease or disorder, the identity of the subject (e.g., age, sex, and/or weight) or host being treated, and the like, but can nevertheless be routinely determined by one of skill in the art. Also, as used herein, a “therapeutically effective amount” of an agent is an amount which results in a beneficial or desired result in a subject as compared to a control. As defined herein, a therapeutically effective amount of an agent may be readily determined by one of ordinary skill by routine methods known in the art. Dosage regimen may be adjusted to provide the optimum therapeutic response.

“Prophylactically effective amount,” as used herein, is intended to include the amount of an oligonucleotide that, when administered to a subject having or predisposed to have a disorder, is sufficient to prevent or ameliorate the disease or one or more symptoms of the disease. Ameliorating the disease includes slowing the course of the disease or reducing the severity of later-developing disease. The “prophylactically effective amount” may vary depending on the oligonucleotide, how the agent is administered, the degree of risk of disease, and the history, age, weight, family history, genetic makeup, the types of preceding or concomitant treatments, if any, and other individual characteristics of the patient to be treated.

A “therapeutically-effective amount” or “prophylactically effective amount” also includes an amount (either administered in a single or in multiple doses) of an oligonucleotide that produces some desired local or systemic effect at a reasonable benefit/risk ratio applicable to any treatment. Oligonucleotides employed in the methods of the present invention may be administered in a sufficient amount to produce a reasonable benefit/risk ratio applicable to such treatment.

A prophylactically effective amount may also refer to, for example, an amount sufficient to, when administered to the subject, including a human, to delay the onset of one or more of the disorders described herein by at least 120 days, for example, at least 6 months, at least 12 months, at least 2 years, at least 3 years, at least 4 years, at least 5 years, at least 10 years or more, when compared with the predicted onset.

For any of the following chemical definitions, a number following an atomic symbol indicates that total number of atoms of that element that are present in a particular chemical moiety. As will be understood, other atoms, such as H atoms, or substituent groups, as described herein, may be present, as necessary, to satisfy the valences of the atoms. For example, an unsubstituted C₂ alkyl group has the formula —CH₂CH₃. When used with the groups defined herein, a reference to the number of carbon atoms includes the divalent carbon in acetal and ketal groups but does not include the carbonyl carbon in acyl, ester, carbonate, or carbamate groups. A reference to the number of oxygen, nitrogen, or sulfur atoms in a heteroaryl group only includes those atoms that form a part of a heterocyclic ring.

When a particular substituent may be present multiple times in the same structure, each instance of the substituent may be independently selected from the list of possible definitions for that substituent.

The term “alkyl,” as used herein, refers to a branched or straight-chain monovalent saturated aliphatic hydrocarbon radical of 1 to 20 carbon atoms (e.g., 1 to 16 carbon atoms, 1 to 10 carbon atoms, 1 to 6 carbon atoms, or 1 to 3 carbon atoms).

An alkylene is a divalent alkyl group. The term “alkenyl,” as used herein, alone or in combination with other groups, refers to a straight chain or branched hydrocarbon residue having a carbon-carbon double bond and having 2 to 20 carbon atoms (e.g., 2 to 16 carbon atoms, 2 to 10 carbon atoms, 2 to 6 carbon atoms, or 2 carbon atoms).

The term “halogen,” as used herein, means a fluorine (fluoro), chlorine (chloro), bromine (bromo), or iodine (iodo) radical.

The term “heteroalkyl,” as used herein, refers to an alkyl group, as defined herein, in which one or more of the constituent carbon atoms have been replaced by nitrogen, oxygen, or sulfur. In some embodiments, the heteroalkyl group can be further substituted with 1, 2, 3, or 4 substituent groups as described herein for alkyl groups. Examples of heteroalkyl groups are an “alkoxy” which, as used herein, refers alkyl-O— (e.g., methoxy and ethoxy). A heteroalkylene is a divalent heteroalkyl group. The term “heteroalkenyl,” as used herein, refers to an alkenyl group, as defined herein, in which one or more of the constituent carbon atoms have been replaced by nitrogen, oxygen, or sulfur. In some embodiments, the heteroalkenyl group can be further substituted with 1, 2, 3, or 4 substituent groups as described herein for alkenyl groups. Examples of heteroalkenyl groups are an “alkenoxy” which, as used herein, refers alkenyl-O—. A heteroalkenylene is a divalent heteroalkenyl group. The term “heteroalkynyl,” as used herein, refers to an alkynyl group, as defined herein, in which one or more of the constituent carbon atoms have been replaced by nitrogen, oxygen, or sulfur. In some embodiments, the heteroalkynyl group can be further substituted with 1, 2, 3, or 4 substituent groups as described herein for alkynyl groups. Examples of heteroalkynyl groups are an “alkynoxy” which, as used herein, refers alkynyl-O—. A heteroalkynylene is a divalent heteroalkynyl group.

The term “hydroxy,” as used herein, represents an —OH group.

The alkyl, heteroalkyl groups may be substituted or unsubstituted. When substituted, there will generally be 1 to 4 substituents present, unless otherwise specified. Substituents include, for example: alkyl (e.g., unsubstituted and substituted, where the substituents include any group described herein, e.g., aryl, halo, hydroxy), aryl (e.g., substituted and unsubstituted phenyl), carbocyclyl (e.g., substituted and unsubstituted cycloalkyl), halo (e.g., fluoro), hydroxyl, heteroalkyl (e.g., substituted and unsubstituted methoxy, ethoxy, or thioalkoxy), heteroaryl, heterocyclyl, amino (e.g., NH₂ or mono- or dialkyl amino), azido, cyano, nitro, or thiol. Aryl, carbocyclyl (e.g., cycloalkyl), heteroaryl, and heterocyclyl groups may also be substituted with alkyl (unsubstituted and substituted such as arylalkyl (e.g., substituted and unsubstituted benzyl)).

Compounds of the invention can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates, or mixtures of diastereoisomeric racemates. The optically active forms can be obtained for example by resolution of the racemates, by asymmetric synthesis or asymmetric chromatography (chromatography with a chiral adsorbent or eluant). That is, certain of the disclosed compounds may exist in various stereoisomeric forms. Stereoisomers are compounds that differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms and represent the configuration of substituents around one or more chiral carbon atoms. Enantiomers of a compound can be prepared, for example, by separating an enantiomer from a racemate using one or more well-known techniques and methods, such as, for example, chiral chromatography and separation methods based thereon. The appropriate technique and/or method for separating an enantiomer of a compound described herein from a racemic mixture can be readily determined by those of skill in the art. “Racemate” or “racemic mixture” means a compound containing two enantiomers, wherein such mixtures exhibit no optical activity; i.e., they do not rotate the plane of polarized light. “Geometric isomer” means isomers that differ in the orientation of substituent atoms in relationship to a carbon-carbon double bond, to a cycloalkyl ring, or to a bridged bicyclic system. Atoms (other than H) on each side of a carbon-carbon double bond may be in an E (substituents are on 25 opposite sides of the carbon-carbon double bond) or Z (substituents are oriented on the same side) configuration. “R,” “S,” “S*,” “R*,” “E,” “Z,” “cis,” and “trans,” indicate configurations relative to the core molecule. Certain of the disclosed compounds may exist in atropisomeric forms. Atropisomers are stereoisomers resulting from hindered rotation about single bonds where the steric strain barrier to rotation is high enough to allow for the isolation of the conformers. The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide 35 of each of the isomers of an isomeric pair using an optically pure acid, amine or alcohol (followed by chromatographic separation and removal of the chiral auxiliary), or resolving an isomeric mixture of either a starting material or a final product using various well known chromatographic methods. When the stereochemistry of a disclosed compound is named or depicted by structure, the named or depicted stereoisomer is at least 60%, 70%, 80%, 90%, 99%, or 99.9% by weight relative to the other stereoisomers. When a single enantiomer is named or depicted by structure, the depicted or named enantiomer is at least 60%, 70%, 80%, 90%, 99%, or 99.9% by weight optically pure. When a single diastereomer is named or depicted by structure, the depicted or named diastereomer is at least 60%, 70%, 80%, 90%, 99%, or 99.9% by weight pure. Percent optical purity is the ratio of the weight of the enantiomer or over the weight of the enantiomer plus the weight of its optical isomer. Diastereomeric purity by weight is the ratio of the weight of one diastereomer or over the weight of all the diastereomers. When the stereochemistry of a disclosed compound is named or depicted by structure, the named or depicted stereoisomer is at least 60%, 70%, 80%, 90%, 99%, or 99.9% by mole fraction pure relative to the other stereoisomers. When a single enantiomer is named or depicted by structure, the depicted or named enantiomer is at least 60%, 70%, 80%, 90%, 99%, or 99.9% by mole fraction pure. When a single diastereomer is named or depicted by structure, the depicted or named diastereomer is at least 60%, 70%, 80%, 90%, 99%, or 99.9% by mole fraction pure. Percent purity by mole fraction is the ratio of the moles of the enantiomer or over the moles of the enantiomer plus the moles of its optical isomer. Similarly, percent purity by moles fraction is the ratio of the moles of the diastereomer or over the moles of the diastereomer plus the moles of its isomer. When a disclosed compound is named or depicted by structure without indicating the stereochemistry, and the compound has at least one chiral center, it is to be understood that the name or structure encompasses either enantiomer of the compound free from the corresponding optical isomer, a racemic mixture of the compound, or mixtures enriched in one enantiomer relative to its corresponding optical isomer. When a disclosed compound is named or depicted by structure without indicating the stereochemistry and has two or more chiral centers, it is to be understood that the name or structure encompasses a diastereomer free of other diastereomers, a number of diastereomers free from other diastereomeric pairs, mixtures of diastereomers, mixtures of diastereomeric pairs, mixtures of diastereomers in which one diastereomer is enriched relative to the other diastereomer(s), or mixtures of diastereomers in which one or more diastereomer is enriched relative to the other diastereomers. The invention embraces all of these forms.

Unless otherwise defined, all technical and scientific terms used herein have the same meaning as commonly understood by one of ordinary skill in the art to which this invention belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control

The details of one or more embodiments of the invention are set forth in the description below. Other features, objects, and advantages of the invention will be apparent from the description and from the claims.

### II. Methods of the Invention

The present invention provides methods of editing a MECP2 polynucleotide, e.g., a MECP2 polynucleotide comprising a single nucleotide polymorphism (SNP) associated with Rett syndrome, and methods for treating or preventing a MECP2-associated disease, e.g., Rett syndrome, in a subject. The methods include contacting the MECP2 polynucleotide with a guide oligonucleotide capable of effecting an adenosine deaminase acting on RNA (ADAR)-mediated adenosine to inosine alteration of the SNP associated with Rett syndrome.

The invention is used to make desired changes in a target sequence, e.g., a MECP2 polynucleotide comprising a SNP associated with Rett syndrome, in a cell or a subject by site-directed editing of nucleotides through the use of an oligonucleotide that is capable of effecting an adenosine deaminase acting on RNA (ADAR)-mediated adenosine to inosine alteration of the SNP. As a result, the target sequence is edited through an adenosine deamination reaction mediated by ADAR, converting adenosines into inosine.

The changes may be in 5′ or 3′ untranslated regions of a target RNA, in splice sites, in exons (changing amino acids in protein translated from the target RNA, changing codon usage or splicing behavior by changing exonic splicing silencers or enhancers, and/or introducing or removing start or stop codons), in introns (changing splicing by altering intronic splicing silencers or intronic splicing enhancers, branch points) and in general in any region affecting RNA stability, structure or functioning. The target RNA sequence may comprise a mutation that one may wish to correct or alter, such as a transition or a transversion.

RNA editing enzymes are known in the art. In some embodiments, the RNA editing enzyme is the adenosine deaminase acting on RNA (ADARs), such as hADARI and hADAR2 in humans or human cells.

Adenosine deaminases acting on RNA (ADARs) catalyze adenosine (A) to inosine (I) editing of RNA that possesses double-stranded (ds) structure. A-to-I RNA editing results in nucleotide substitution, because I is recognized as G instead of A both by ribosomes and by RNA polymerases. A-to-I substitution can also cause dsRNA destabilization, as I:U mismatch base pairs are less stable than A:U base pairs. A-to-I editing occurs with both viral and cellular RNAs, and affects a broad range of biological processes. These include virus growth and persistence, apoptosis and embryogenesis, neurotransmitter receptor and ion channel function, pancreatic cell function, and post-transcriptional gene regulation by microRNAs. Biochemical processes that provide a framework for understanding the physiologic changes following ADAR-catalyzed A-to-I (=G) editing events include mRNA translation by changing codons and hence the amino acid sequence of proteins; pre-mRNA splicing by altering splice site recognition sequences; RNA stability by changing sequences involved in nuclease recognition; genetic stability in the case of RNA virus genomes by changing sequences during viral RNA replication; and RNA-structure-dependent activities such as microRNA production or targeting or protein-RNA interactions.

Three human ADAR genes are known, of which two encode active deaminases (ADAR1 and ADAR2). Human ADAR3 (hADAR3) has been described in the prior art, but reportedly has no deaminase activity. Alternative promoters together with alternative splicing give rise to two protein size forms of ADAR1: an interferon-inducible ADAR1-p150 deaminase that binds dsRNA and Z-DNA, and a constitutively expressed ADAR1-p110 deaminase. ADAR2, like ADAR1-p110, is constitutively expressed and binds dsRNA. It is known that only the longer isoform of ADAR1 is capable of binding to the Z-DNA structure that can be comprised in the recruiting portion of the oligonucleotide construct according to the invention. Consequently, the level of the 150 kDa isoform present in the cell may be influenced by interferon, particularly interferon-gamma (IFN-gamma). hADARI is also inducible by TNF-alpha. This provides an opportunity to develop combination therapy, whereby interferon-gamma or TNF-alpha and oligonucleotide constructs comprising Z-DNA as recruiting portion according to the invention are administered to a patient either as a combination product, or as separate products, either simultaneously or subsequently, in any order. Certain disease conditions may already coincide with increased IFN-gamma or TNF-alpha levels in certain tissues of a patient, creating further opportunities to make editing more specific for diseased tissues.

Recruiting ADAR to specific sites of selected transcripts and deamination of adenosine regardless of neighboring bases holds great promise for the treatment of disease. In some embodiments, the oligonucleotide that is capable of effecting an adenosine deaminase acting on RNA (ADAR)-mediated adenosine to inosine alteration of the SNP, e.g., a guide oligonucleotide as described herein, further comprises an ADAR-recruiting domain. In some embodiments, the ADAR-recruiting domain comprises nucleotide sequences that may be covalently linked to the oligonucleotides for use in the methods of the instant invention and may form stem-loop structures that act as recruitment and binding regions for the ADAR enzyme. Oligonucleotides including such ADAR-recruiting domains may be referred to as “axiomer AONs” or “self-looping AONs.” The ADAR-recruiting domain portion may act to recruit an endogenous ADAR enzyme present in the cell. Such ADAR-recruiting domains do not require conjugated entities or presence of modified recombinant ADAR enzymes. Alternatively, the ADAR-recruiting portion may act to recruit a recombinant ADAR fusion protein that has been delivered to a cell or to a subject via an expression vector construct including a polynucleotide encoding an ADAR fusion protein. Such ADAR-fusion proteins may include the deaminase domain of ADAR1 or ADAR2 enzymes fused to another protein, e.g., to the MS2 bacteriophage coat protein. An ADAR-recruiting domain may be a nucleotide sequence based on a natural substrate (e.g., the GluR2 receptor pre-mRNA; such as a GluR2 ADAR-recruiting domain), a Z-DNA structure, or a domain known to recruit another protein which is part of an ADAR fusion protein, e.g., an MS2 ADAR-recruiting domain known to be recognized by the dsRNA binding regions of ADAR. A stem-loop structure of an ADAR-recruiting domain can be an intermolecular stem-loop structure, formed by two separate nucleic acid strands, or an intramolecular stem loop structure, formed within a single nucleic acid strand.

In some embodiments, the ADAR is endogenously expressed in a cell. The cell is selected from the group consisting of a bacterial cell, a eukaryotic cell, a mammalian cell, and a human cell. In principle the invention can be used with cells from any mammalian species, but it is preferably used with a human cell.

The oligonucleotide capable of effecting an adenosine deaminase acting on RNA (ADAR)-mediated adenosine to inosine alteration of the SNP, e.g., a guide oligonucleotide as described herein, comprises a nucleic acid sequence complementary to the MECP2 mRNA encoding the SNP associated with Rett syndrome. In some embodiments, the guide oligonucleotides are complementary to target mRNA with the exception of at least one mismatch. The oligonucleotide includes a mismatch opposite the target adenosine.

Once the oligonucleotide hybridizes to the target mRNA sequence, it forms a double-stranded RNA structure, which can be recognized by ADAR, and facilitates the recruitment of ADAR to the target sequence. As a result, ADAR can catalyze the deamination reaction of the specific adenosine in the SNP associated with Rett syndrome into an inosine.

As used herein, the phrase “SNP associated with Rett syndrome” refers to any SNPs that are associated with the onset or development of Rett syndrome. Exemplary SNPs associated with Rett syndrome may include, but are not limited to, any single nucleotide change in the MECP2 polynucleotide resulting in a pathogenic amino acid at positions 255, 168, 294 and/or 270 of the MECP2 protein. These mutations have been demonstrated to be pathogenic and are associated with the late-onset of Rett syndrome.

In some embodiments, the SNP associated with Rett syndrome is rs61749721, a SNP commonly referred to as the R255X variant (or mutation) based on the potential change from arginine (encoded by wild type allele) to a premature stop codon (encoded by the rs61749721 allele) at position 255 of the MECP2 protein. This SNP is also referred to as the R255X variant (or, mutation) based on the change from arginine to a premature stop codon at position 255 of the MECP2 protein. Upon successful editing by the methods of the invention, the allele is deaminated by ADAR, and this ADAR-mediated adenosine to inosine alteration substitutes the pathogenic amino acid, a premature stop codon, at position 255 of the MECP2 protein with a tryptophan, thereby removing the pathogenic or disease causing mutation in MECP2 protein.

In some embodiments, the SNP associated with Rett syndrome is rs61748421, a SNP commonly referred to as the R168X variant (or mutation) based on the potential change from arginine (encoded by rs61748421(C) allele) to a premature stop codon (encoded by the rs61748421(T) allele) at position 168 of the MECP2 protein. Upon successful editing by the methods of the invention, the rs61748421(A) allele is deaminated and converted to the rs61748421(G) allele by ADAR, and this ADAR-mediated adenosine to inosine alteration substitutes the pathogenic amino acid, a premature stop codon, at position 168 of the MECP2 protein with a tryptophan, thereby removing the pathogenic or disease causing mutation in MECP2 protein.

In some embodiments, the SNP associated with Rett syndrome is rs61751362, a SNP commonly referred to as the R294X variant (or mutation) based on the potential change from arginine (encoded by rs61751362(C) allele) to a premature stop codon (encoded by the rs61751362(T) allele) at position 294 of the MECP2 protein. Upon successful editing by the methods of the invention, the rs61751362(A) allele) is deaminated and converted to the rs61751362(G) allele by ADAR, and this ADAR-mediated adenosine to inosine alteration substitutes the pathogenic amino acid, a premature stop codon, at position 294 of the MECP2 protein with a tryptophan, thereby removing the pathogenic or disease causing mutation in MECP2 protein.

In some embodiments, the SNP associated with Rett syndrome is rs61750240, a SNP commonly referred to as the R270X variant (or mutation) based on the potential change from arginine (encoded by rs61750240(C) allele) to a premature stop codon (encoded by the rs61750240(T) allele) at position 270 of the MECP2 protein. Upon successful editing by the methods of the invention, the rs61748421(A) allele) is deaminated and converted to the rs61748421(G) allele by ADAR, and this ADAR-mediated adenosine to inosine alteration substitutes the pathogenic amino acid, a premature stop codon, at position 270 of the MECP2 protein with a tryptophan, thereby removing the pathogenic or disease causing mutation in MECP2 protein.

The methods of the present invention can be used with cells from any organ, e.g. skin, lung, heart, kidney, liver, pancreas, gut, muscle, gland, eye, brain, blood and the like. The invention is particularly suitable for modifying sequences in cells, tissues or organs implicated in a diseased state of a (human) subject. Such cells include but are not limited to brain cells or neurons, e.g., neurons near the base of the brain (i.e., midbrain or the substantia nigra), neurons from the brainstem, neurons in the striatum, neurons in the cortex, or neurons in the olfactory bulb.

The methods of the invention can also be used with mammalian cells which are not naturally present in an organism e.g. with a cell line or with an embryonic stem (ES) cell. The methods of the invention can be used with various types of stem cells, including pluripotent stem cells, totipotent stem cells, embryonic stem cells, induced pluripotent stem cells, etc.

The cells can be located in vitro or in vivo. One advantage of the invention is that it can be used with cells in situ in a living organism, but it can also be used with cells in culture. In some embodiments cells are treated ex vivo and are then introduced into a living organism (e.g. re-introduced into an organism from whom they were originally derived). In some embodiments, the cell is contacted in vivo. In other embodiments, the cell is ex vivo.

The methods of invention can also be used to edit target RNA sequences in cells within a so-called organoid. Organoids are self-organized three-dimensional tissue structures derived from stem cells. Such cultures can be crafted to replicate much of the complexity of an organ, or to express selected aspects of it like producing only certain types of cells (Lancaster & Knoblich, Science 2014, vol. 345 no. 6194 1247125). In a therapeutic setting they are useful because they can be derived in vitro from a patient's cells, and the organoids can then be re-introduced to the patient as autologous material which is less likely to be rejected than a normal transplant. Thus, according to another preferred embodiment, the invention may be practised on organoids grown from tissue samples taken from a patient (e.g. from their gastrointestinal tract; see Sala et al. J Surg Res. 2009; 156(2):205-12, and Sato et al. Gastroenterology 201 1; 141: 1762-72). Upon RNA editing in accordance with the invention, the organoids, or stem cells residing within the organoids, may be used to transplant back into the patient to ameliorate organ function.

In some embodiments, the cells to be treated have a genetic mutation. The mutation may be heterozygous or homozygous. The invention can be used to modify point mutations, for example, to correct a G to A mutation. In other embodiments, the cells to be treated do not have a genetic mutation. The invention can be used to create point mutations, for example, to generate a A to G mutation.

Accordingly, the invention is not limited to correcting mutations, as it may instead be useful to change a wild-type sequence into a mutated sequence by applying oligonucleotides according to the invention. One example where it may be advantageous to modify a wild-type adenosine is to bring about skipping of an exon, for example by modifying an adenosine that happens to be a branch site required for splicing of said exon. Another example is where the adenosine defines or is part of a recognition sequence for protein binding, or is involved in secondary structure defining the stability of the mRNA. In some embodiments, however, the invention is used in the opposite way by introducing a disease-associated mutation into a cell line or an animal, in order to provide a useful research tool for the disease in question. As an example of creating a disease model for research purposes, an oligonucleotide sequence described herein provides for the recruitment of editing activity in a human cell to create a mutation in MECP2, e.g., a R255X, R168X, R294X, and/or R270X mutation, that forms the basis for the onset of Rett syndrome. As a result, the invention can be used to provide research tools for diseases, to introduce new mutations which are less deleterious than an existing mutation.

A mutation to be reverted through RNA editing may have arisen on the level of the chromosome or some other form of DNA, such as mitochondrial DNA, or RNA, including pre-mRNA, ribosomal RNA or mitochondrial RNA. A change to be made may be in a target RNA of a pathogen, including fungi, yeasts, parasites, kinetoplastids, bacteria, phages, viruses etc, with which the cell or subject has been infected. Subsequently, the editing may take place on the RNA level on a target sequence inside such cell, subject or pathogen. Certain pathogens, such as viruses, release their nucleic acid, DNA or RNA into the cell of the infected host (cell). Other pathogens reside or circulate in the infected host. The oligonucleotide constructs of the invention may be used to edit target RNA sequences residing in a cell of the infected eukaryotic host, or to edit a RNA sequence inside the cell of a pathogen residing or circulating in the eukaryotic host, as long as the cells where the editing is to take place contain an editing entity compatible with the oligonucleotide construct administered thereto.

Without wishing to be bound be theory, the RNA editing through ADAR1 and ADAR2 is thought to take place on pre-mRNAs in the nucleus, during transcription or splicing. Editing of mitochondrial RNA codons or non-coding sequences in mature mRNAs is not excluded.

Deamination of an adenosine using the oligonucleotides disclosed herein includes any level of adenosine deamination, e.g., at least 1 deaminated adenosine within a target sequence (e.g., at least, 1, 2, 3, or more deaminated adenosines in a target sequence).

Adenosine deamination may be assessed by a decrease in an absolute or relative level of adenosines within a target sequence compared with a control level. The control level may be any type of control level that is utilized in the art, e.g., pre-dose baseline level, or a level determined from a similar subject, cell, or sample that is untreated or treated with a control (such as, e.g., buffer only control or inactive agent control).

Because the enzymatic activity of ADAR converts adenosines to inosines, adenosine deamination can alternatively be assessed by an increase in an absolute or relative level of inosines within a target sequence compared with a control level. Similarly, the control level may be any type of control level that is utilized in the art, e.g., pre-dose baseline level, or a level determined from a similar subject, cell, or sample that is untreated or treated with a control (such as, e.g., buffer only control or inactive agent control).

The levels of adenosines and/or inosines within a target sequence can be assessed using any of the methods known in the art for determining the nucleotide composition of a polynucleotide sequence. For example, the relative or absolute levels of adenosines or inosines within a target sequence can be assessed using nucleic acid sequencing technologies including but not limited to Sanger sequencing methods, Next Generation Sequencing (NGS; e.g., pyrosequencing, sequencing by reversible terminator chemistry, sequencing by ligation, and real-time sequencing) such as those offered on commercially available platforms (e.g., Illumina, Qiagen, Pacific Biosciences, Thermo Fisher, Roche, and Oxford Nanopore Technologies). Clonal amplification of target sequences for NGS may be performed using real-time polymerase chain reaction (also known as qPCR) on commercially available platforms from Applied Biosystems, Roche, Stratagene, Cepheid, Eppendorf, or Bio-Rad Laboratories. Additionally or alternatively, emulsion PCR methods can be used for amplification of target sequences using commercially available platforms such as Droplet Digital PCR by Bio-Rad Laboratories.

In certain embodiments, surrogate markers can be used to detect adenosine deamination within a target sequence. For example, effective treatment of a subject having a genetic disorder involving G-to-A mutations with an oligonucleotide of the present disclosure, as demonstrated by an acceptable diagnostic and monitoring criteria can be understood to demonstrate a clinically relevant adenosine deamination. In certain embodiments, the methods include a clinically relevant adenosine deamination, e.g., as demonstrated by a clinically relevant outcome after treatment of a subject with an oligonucleotide of the present disclosure.

Adenosine deamination in a gene of interest may be manifested by an increase or decrease in the levels of mRNA expressed by a first cell or group of cells (such cells may be present, for example, in a sample derived from a subject) in which a gene of interest is transcribed and which has or have been treated (e.g., by contacting the cell or cells with an oligonucleotide of the present disclosure, or by administering an oligonucleotide of the invention to a subject in which the cells are or were present) such that the expression of the gene of interest is increased or decreased, as compared to a second cell or group of cells substantially identical to the first cell or group of cells but which has not or have not been so treated (control cell(s) not treated with an oligonucleotide or not treated with an oligonucleotide targeted to the gene of interest). The degree of increase or decrease in the levels of mRNA of a gene of interest may be expressed in terms of:

$$\frac{( mRNA in control cells ) - ( mRNA in treated cells )}{( mRNA in control cells )} \times 100 \%$$

In other embodiments, change in the levels of a gene may be assessed in terms of a reduction of a parameter that is functionally linked to the expression of a gene of interest, e.g., protein expression of the gene of interest or signaling downstream of the protein. A change in the levels of the gene of interest may be determined in any cell expressing the gene of interest, either endogenous or heterologous from an expression construct, and by any assay known in the art.

A change in the level of expression of a gene of interest may be manifested by an increase or decrease in the level of the protein produced by the gene of interest that is expressed by a cell or group of cells (e.g., the level of protein expressed in a sample derived from a subject). As explained above, for the assessment of mRNA suppression, the change in the level of protein expression in a treated cell or group of cells may similarly be expressed as a percentage of the level of protein in a control cell or group of cells.

A control cell or group of cells that may be used to assess the change in the expression of a gene of interest includes a cell or group of cells that has not yet been contacted with an oligonucleotide of the present disclosure. For example, the control cell or group of cells may be derived from an individual subject (e.g., a human or animal subject) prior to treatment of the subject with an oligonucleotide.

The level of mRNA of a gene of interest that is expressed by a cell or group of cells may be determined using any method known in the art for assessing mRNA expression. In one embodiment, the level of expression of a gene of interest in a sample is determined by detecting a transcribed polynucleotide, or portion thereof, e.g., mRNA of the gene of interest. RNA may be extracted from cells using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating mRNA of the gene of interest may be detected using methods the described in PCT Publication WO2012/177906, the entire contents of which are hereby incorporated herein by reference. In some embodiments, the level of expression of the gene of interest is determined using a nucleic acid probe. The term “probe,” as used herein, refers to any molecule that is capable of selectively binding to a specific sequence, e.g. to an mRNA or polypeptide. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, Southern or northern analyses, polymerase chain reaction (PCR) analyses, and probe arrays. One method for the determination of mRNA levels involves contacting the isolated mRNA with a nucleic acid molecule (probe) that can hybridize to the mRNA of a gene of interest. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an AFFYMETRIX gene chip array. A skilled artisan can readily adapt known mRNA detection methods for use in determining the level of mRNA of a gene of interest.

An alternative method for determining the level of expression of a gene of interest in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the invention, the level of expression of a gene of interest is determined by quantitative fluorogenic RT-PCR (i.e., the TAQMAN™ System) or the DUAL-GLO® Luciferase assay.

The expression levels of mRNA of a gene of interest may be monitored using a membrane blot (such as used in hybridization analysis such as northern, Southern, dot, and the like), or microwells, sample tubes, gels, beads or fibers (or any solid support including bound nucleic acids). See U.S. Pat. Nos. 5,770,722; 5,874,219; 5,744,305; 5,677,195; and 5,445,934, which are incorporated herein by reference. The determination of gene expression level may also include using nucleic acid probes in solution.

In some embodiments, the level of mRNA expression is assessed using branched DNA (bDNA) assays or real time PCR (qPCR). The use of this PCR method is described and exemplified in the Examples presented herein. Such methods can also be used for the detection of nucleic acids of the gene of interest.

The level of protein produced by the expression of a gene of interest may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of proteins produced by the gene of interest. Additionally, the above assays may be used to report a change in the mRNA sequence of interest that results in the recovery or change in protein function thereby providing a therapeutic effect and benefit to the subject, treating a disorder in a subject, and/or reducing of symptoms of a disorder in the subject.

### Methods of Treatment

The present invention also include methods of treating or preventing a MECP2-associated disease or disorder, e.g., Rett syndrome. For example, the methods of the invention may be used to treat or prevent any MECP2-associated disorders which may be caused by a guanosine to adenosine mutation, the introduction of a premature stop codon, or expression of an undesired protein. In some embodiments, the oligonucleotides for use in the methods of the invention, when introduced to a cell or a subject, can result in correction of a guanosine to adenosine mutation. In some embodiments, the oligonucleotides for use in the methods of the invention can result in turning off of a premature stop codon so that a desired protein is expressed. In some embodiments, the oligonucleotides for use in the methods of the invention can result in inhibition of expression of an undesired protein.

In one aspect, the present invention is directed to a method of treating Rett syndrome in a subject in need thereof. The method comprises identifying a subject with a single nucleotide polymorphism (SNP) associated with Rett syndrome in a MECP2 polynucleotide; and contacting the MECP2 polynucleotide in a cell of the subject with a guide oligonucleotide capable of effecting an adenosine deaminase acting on RNA (ADAR)-mediated adenosine to inosine alteration of the SNP associated with Rett syndrome, thereby treating the subject.

In another aspect, the present invention is directed to a method of treating Rett syndrome in a subject in need thereof. The method comprises identifying a subject with a single nucleotide polymorphism (SNP) associated with Rett syndrome in a MECP2 polynucleotide; contacting the MECP2 polynucleotide in a cell with a guide oligonucleotide capable of effecting an adenosine deaminase acting on RNA (ADAR)-mediated adenosine to inosine alteration of the SNP associated with Rett syndrome, and administering the cell to the subject, thereby treating the subject.

In some embodiments, the subject is a human subject.

The methods of the invention thus may include a step of identifying a subject with a single nucleotide polymorphism (SNP) associated with Rett syndrome in a MECP2 polynucleotide. Specifically, the methods of the invention include a step of identifying the presence of the desired nucleotide change or SNPs in the target RNA sequence, thereby verifying that the target RNA sequence has the disease causing mutations to be corrected or edited. This step will typically involve sequencing of the relevant part of the target RNA sequence, or a cDNA copy thereof (or a cDNA copy of a splicing product thereof, in case the target RNA is a pre-mRNA), and the sequence change can thus be easily verified. Alternatively the modifications may be assessed on the level of the protein (length, glycosylation, function or the like), or by some functional read-out.

The methods disclosed herein also include contacting the MECP2 polynucleotides with a single nucleotide polymorphism (SNP) associated with Rett syndrome in a cell or a subject (including a subject identified as being in need of such treatment, or a subject suspected of being at risk of disease and in need of such treatment) with a guide oligonucleotide capable of effecting an adenosine deaminase acting on RNA (ADAR)-mediated adenosine to inosine alteration of the SNP associated with Rett syndrome, as described herein.

The guide oligonucleotides for use in the methods of the invention are designed to specifically target the MECP2 gene of a subject (e.g., a human patient) in need thereof, and are capable of effecting an ADAR-mediated adenosine to inosine alteration in the SNPs associated with Rett syndrome in the MECP2 gene. In some embodiments, the guide oligonucleotides are capable of recruiting the ADAR to the target mRNA, which then catalyze deamination of target adenosines in the target mRNA. Such treatment will be suitably introduced to a subject, particularly a human subject, suffering from, having, susceptible to, or at risk for developing Rett syndrome. The compositions disclosed herein may be also used in the treatment of any other disorders in which Rett syndrome may be implicated.

In one embodiment, the invention provides a method of monitoring treatment progress. The method includes the step of determining a level of diagnostic marker (Marker) (e.g., SNP associated with Rett syndrome) or diagnostic measurement (e.g., screen, assay) in a subject suffering from or susceptible to developing Rett syndrome, or symptoms associated with Rett syndrome in which the subject has been administered a therapeutic amount of a composition disclosed herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment. Other methods of diagnostic measurement include, but are not limited to, observing one or more of the signs and symptoms of Rett syndrome disclosed herein, during the child's early growth and development, and conducting ongoing evaluations of the child's physical and neurological status. Examples of main diagnostic criteria or symptoms include partial or complete loss of acquired purposeful hand skills, partial or complete loss of acquired spoken language, repetitive hand movements (such has hand wringing or squeezing, clapping or rubbing), and gait abnormalities, including toe-walking or an unsteady, wide-based, stiff-legged walk. Supportive criteria are not required for a diagnosis of Rett syndrome but may occur in some individuals. In addition, these symptoms which vary in severity from child to child may not be observed in very young girls but may develop with age. A child with supportive criteria but none of the essential criteria does not have Rett syndrome. Supportive criteria include scoliosis, teeth-grinding, small cold hands and feet in relation to height, abnormal sleep patterns, abnormal muscle tone, inappropriate laughing or screaming, intense eye communication, and diminished response to pain. In addition to the main diagnostic criteria, a number of specific conditions help rule out a diagnosis of Rett syndrome. These are referred to as exclusion criteria. Children with any one of the following criteria do not have Rett syndrome: brain injury secondary to trauma, neurometabolic disease, severe infection that causes neurological problems; and grossly abnormal psychomotor development in the first 6 months of life.

In some embodiments, cells are obtained from the subject and contacted with an oligonucleotide composition of the invention as provided herein. In some embodiments, the cell is autologous, allogenic, or xenogenic to the subject. In some embodiments, cells removed from a subject and contacted ex vivo with an oligonucleotide composition of the invention are re-introduced into the subject, optionally after the desired genomic modification has been effected or detected in the cells.

In some embodiments, the oligonucleotide for use in the methods of the present disclosure is introduced to a subject such that the oligonucleotide is delivered to a specific site within the subject. The change in the expression of the gene of interest may be assessed using measurements of the level or change in the level of mRNA or protein produced by the gene of interest in a sample derived from a specific site within the subject.

In other embodiments, the oligonucleotide is introduced into the cell or the subject in an amount and for a time effective to result in one of (or more, e.g., two or more, three or more, four or more of: (a) decrease the number of adenosines within a target sequence of the gene of interest, (b) decrease the number of pathogenic mutations in the target protein, e.g., MECP2, (c) delayed onset of Rett syndrome, (d) increased survival of subject, (e) recovery or change in protein function, and (f) reduction in one or more of symptoms related to Rett syndrome, such as loss of the purposeful use of hands and the ability to speak, problems crawling or walking and diminished eye contact, compulsive hand movements such as wringing and washing, apraxia, autistic-like behaviors, walking on the toes, sleep problems, a wide-based gait, teeth grinding and difficulty chewing, slowed growth, seizures, cognitive disabilities, and breathing difficulties while awake such as hyperventilation, apnea (breath holding), and air swallowing.

Treating disorders associated with G-to-A mutations can also result in a decrease in the mortality rate of a population of treated subjects in comparison to an untreated population. For example, the mortality rate is decreased by more than 2% (e.g., more than 5%, 10%, or 25%). A decrease in the mortality rate of a population of treated subjects may be measured by any reproducible means, for example, by calculating for a population the average number of disease-related deaths per unit time following initiation of treatment with a compound or pharmaceutically acceptable salt of a compound described herein. A decrease in the mortality rate of a population may also be measured, for example, by calculating for a population the average number of disease-related deaths per unit time following completion of a first round of treatment with a compound or pharmaceutically acceptable salt of a compound described herein.

A. Methods of Administration

The delivery of an oligonucleotide for use in the methods of the invention to a cell e.g., a cell within a subject, such as a human subject (e.g., a subject in need thereof, such as a subject having a Rett syndrome) can be achieved in a number of different ways. For example, delivery may be performed by contacting a cell with an oligonucleotide of the invention either in vitro or in vivo. In vivo delivery may also be performed directly by administering a composition including an oligonucleotide to a subject. Alternatively, in vivo delivery may be performed indirectly by administering one or more vectors that encode and direct the expression of the oligonucleotide. Combinations of in vitro and in vivo methods of contacting a cell are also possible. Contacting a cell may be direct or indirect. Furthermore, contacting a cell may be accomplished via a targeting ligand, including any ligand described herein or known in the art. In some embodiments, the targeting ligand is a carbohydrate moiety, e.g., a GalNAc₃ ligand, or any other ligand that directs the oligonucleotide to a site of interest, for example, the central nervous system, such as, the midbrain, e.g., the substantia nigra; the striatum; the brainstem; the cortex; or the forebrain, e.g., the olfactory bulb.

Contacting of a cell with an oligonucleotide may be done in vitro or in vivo. Known methods can be adapted for use with an oligonucleotide of the invention (see e.g., Akhtar S. and Julian R L., (1992) Trends Cell. Biol. 2(5):139-144 and WO94/02595, which are incorporated herein by reference in their entireties). For in vivo delivery, factors to consider in order to deliver an oligonucleotide molecule include, for example, biological stability of the delivered molecule, prevention of non-specific effects, and accumulation of the delivered molecule in the target tissue. The non-specific effects of an oligonucleotide can be minimized by local administration, for example, by direct injection or implantation into a tissue or topically administering the preparation. Local administration to a treatment site maximizes local concentration of the agent, limits the exposure of the agent to systemic tissues that can otherwise be harmed by the agent or that can degrade the agent, and permits a lower total dose of the oligonucleotide molecule to be administered.

For administering an oligonucleotide systemically for the treatment of a disease, the oligonucleotide can include alternative nucleobases, alternative sugar moieties, and/or alternative internucleoside linkages, or alternatively delivered using a drug delivery system; both methods act to prevent the rapid degradation of the oligonucleotide by endo- and exo-nucleases in vivo. Modification of the oligonucleotide or the pharmaceutical carrier can also permit targeting of the oligonucleotide composition to the target tissue and avoid undesirable off-target effects. Oligonucleotide molecules can be modified by chemical conjugation to lipophilic groups such as cholesterol to enhance cellular uptake and prevent degradation. In an alternative embodiment, the oligonucleotide can be delivered using drug delivery systems such as a nanoparticle, a lipid nanoparticle, a polyplex nanoparticle, a lipoplex nanoparticle, a dendrimer, a polymer, liposomes, or a cationic delivery system. Positively charged cationic delivery systems facilitate binding of an oligonucleotide molecule (negatively charged) and also enhance interactions at the negatively charged cell membrane to permit efficient uptake of an oligonucleotide by the cell. Cationic lipids, dendrimers, or polymers can either be bound to an oligonucleotide, or induced to form a vesicle or micelle that encases an oligonucleotide. The formation of vesicles or micelles further prevents degradation of the oligonucleotide when administered systemically. In general, any methods of delivery of nucleic acids known in the art may be adaptable to the delivery of the oligonucleotides of the invention. Methods for making and administering cationic oligonucleotide complexes are well within the abilities of one skilled in the art (see e.g., Sorensen, D R., et al. (2003) J. Mol. Biol 327:761-766; Verma, U N. et al., (2003) Clin. Cancer Res. 9:1291-1300; Arnold, A S et al., (2007) J. Hypertens. 25:197-205, which are incorporated herein by reference in their entirety). Some non-limiting examples of drug delivery systems useful for systemic delivery of oligonucleotides include DOTAP (Sorensen, D R., et al (2003), supra; Verma, U N. et al., (2003), supra), Oligofectamine, “solid nucleic acid lipid particles” (Zimmermann, T S. et al., (2006) Nature 441:111-114), cardiolipin (Chien, P Y. et al., (2005) Cancer Gene Ther. 12:321-328; Pal, A. et al., (2005) Int J. Oncol. 26:1087-1091), polyethyleneimine (Bonnet M E. et al., (2008) Pharm. Res. August 16 Epub ahead of print; Aigner, A. (2006) J. Biomed. Biotechnol. 71659), Arg-Gly-Asp (RGD) peptides (Liu, S. (2006) Mol. Pharm. 3:472-487), and polyamidoamines (Tomalia, D A. et al., (2007) Biochem. Soc. Trans. 35:61-67; Yoo, H. et al., (1999) Pharm. Res. 16:1799-1804). In some embodiments, an oligonucleotide forms a complex with cyclodextrin for systemic administration. Methods for administration and pharmaceutical compositions of oligonucleotides and cyclodextrins can be found in U.S. Pat. No. 7,427,605, which is herein incorporated by reference in its entirety. In some embodiments the oligonucleotides of the invention are delivered by polyplex or lipoplex nanoparticles. Methods for administration and pharmaceutical compositions of oligonucleotides and polyplex nanoparticles and lipoplex nanoparticles can be found in U.S. Patent Application Nos. 2017/0121454; 2016/0369269; 2016/0279256; 2016/0251478; 2016/0230189; 2015/0335764; 2015/0307554; 2015/0174549; 2014/0342003; 2014/0135376; and 2013/0317086, which are herein incorporated by reference in their entirety.

i. Membranous Molecular Assembly Delivery Methods

Oligonucleotides for use in the methods of the invention can also be delivered using a variety of membranous molecular assembly delivery methods including polymeric, biodegradable microparticle, or microcapsule delivery devices known in the art. For example, a colloidal dispersion system may be used for targeted delivery an oligonucleotide agent described herein. Colloidal dispersion systems include macromolecule complexes, nanocapsules, microspheres, beads, and lipid-based systems including oil-in-water emulsions, micelles, mixed micelles, and liposomes. Liposomes are artificial membrane vesicles that are useful as delivery vehicles in vitro and in vivo. It has been shown that large unilamellar vesicles (LUV), which range in size from 0.2-4.0 μm can encapsulate a substantial percentage of an aqueous buffer containing large macromolecules. Liposomes are useful for the transfer and delivery of active ingredients to the site of action. Because the liposomal membrane is structurally similar to biological membranes, when liposomes are applied to a tissue, the liposomal bilayer fuses with bilayer of the cellular membranes. As the merging of the liposome and cell progresses, the internal aqueous contents that include the oligonucleotide are delivered into the cell where the oligonucleotide can specifically bind to a target RNA and can mediate ADAR-mediated RNA editing. In some cases, the liposomes are also specifically targeted, e.g., to direct the oligonucleotide to particular cell types. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

A liposome containing an oligonucleotide can be prepared by a variety of methods. In one example, the lipid component of a liposome is dissolved in a detergent so that micelles are formed with the lipid component. For example, the lipid component can be an amphipathic cationic lipid or lipid conjugate. The detergent can have a high critical micelle concentration and may be nonionic. Exemplary detergents include cholate, CHAPS, octylglucoside, deoxycholate, and lauroyl sarcosine. The oligonucleotide preparation is then added to the micelles that include the lipid component. The cationic groups on the lipid interact with the oligonucleotide and condense around the oligonucleotide to form a liposome. After condensation, the detergent is removed, e.g., by dialysis, to yield a liposomal preparation of oligonucleotide.

If necessary, a carrier compound that assists in condensation can be added during the condensation reaction, e.g., by controlled addition. For example, the carrier compound can be a polymer other than a nucleic acid (e.g., spermine or spermidine). The pH can also be adjusted to favor condensation.

Methods for producing stable polynucleotide delivery vehicles, which incorporate a polynucleotide/cationic lipid complex as a structural component of the delivery vehicle, are further described in, e.g., WO 96/37194, the entire contents of which are incorporated herein by reference. Liposome formation can also include one or more aspects of exemplary methods described in Feigner, P. L. et al., (1987) Proc. Natl. Acad. Sci. USA 8:7413-7417; U.S. Pat. Nos. 4,897,355; 5,171,678; Bangham et al., (1965) M. Mol. Biol. 23:238; Olson et al., (1979) Biochim. Biophys. Acta 557:9; Szoka et al., (1978) Proc. Natl. Acad. Sci. 75: 4194; Mayhew et al., (1984) Biochim. Biophys. Acta 775:169; Kim et al., (1983) Biochim. Biophys. Acta 728:339; and Fukunaga et al., (1984) Endocrinol. 115:757. Commonly used techniques for preparing lipid aggregates of appropriate size for use as delivery vehicles include sonication and freeze-thaw plus extrusion (see, e.g., Mayer et al., (1986) Biochim. Biophys. Acta 858:161. Microfluidization can be used when consistently small (50 to 200 nm) and relatively uniform aggregates are desired (Mayhew et al., (1984) Biochim. Biophys. Acta 775:169. These methods are readily adapted to packaging oligonucleotide preparations into liposomes.

Liposomes fall into two broad classes. Cationic liposomes are positively charged liposomes which interact with the negatively charged nucleic acid molecules to form a stable complex. The positively charged nucleic acid/liposome complex binds to the negatively charged cell surface and is internalized in an endosome. Due to the acidic pH within the endosome, the liposomes are ruptured, releasing their contents into the cell cytoplasm (Wang et al. (1987) Biochem. Biophys. Res. Commun., 147:980-985).

Liposomes, which are pH-sensitive or negatively charged, entrap nucleic acids rather than complex with them. Since both the nucleic acid and the lipid are similarly charged, repulsion rather than complex formation occurs. Nevertheless, some nucleic acid is entrapped within the aqueous interior of these liposomes. pH sensitive liposomes have been used to deliver nucleic acids encoding the thymidine kinase gene to cell monolayers in culture. Expression of the exogenous gene was detected in the target cells (Zhou et al. (1992) Journal of Controlled Release, 19:269-274).

One major type of liposomal composition includes phospholipids other than naturally-derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Examples of other methods to introduce liposomes into cells in vitro and in vivo include U.S. Pat. Nos. 5,283,185; 5,171,678; WO 94/00569; WO 93/24640; WO 91/16024; Feigner, (1994) J. Biol. Chem. 269:2550; Nabel, (1993) Proc. Natl. Acad. Sci. 90:11307; Nabel, (1992) Human Gene Ther. 3:649; Gershon, (1993) Biochem. 32:7143; and Strauss, (1992) EMBO J. 11:417.

Non-ionic liposomal systems have also been examined to determine their utility in the delivery of drugs to the skin, in particular systems including non-ionic surfactant and cholesterol. Non-ionic liposomal formulations including NOVASOME™ I (glyceryl dilaurate/cholesterol/polyoxyethylene-10-stearyl ether) and NOVASOME™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporine A into different layers of the skin (Hu et al., (1994) S.T.P. Pharma. Sci., 4(6):466).

Liposomes may also be sterically stabilized liposomes, including one or more specialized lipids that result in enhanced circulation lifetimes relative to liposomes lacking such specialized lipids. Examples of sterically stabilized liposomes are those in which part of the vesicle-forming lipid portion of the liposome (A) includes one or more glycolipids, such as monosialoganglioside G_{M1}, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., (1987) FEBS Letters, 223:42; Wu et al., (1993) Cancer Research, 53:3765).

Various liposomes including one or more glycolipids are known in the art. Papahadjopoulos et al. (Ann. N.Y. Acad. Sci., (1987), 507:64) reported the ability of monosialoganglio side G^{M1}, galactocerebroside sulfate, and phosphatidylinositol to improve blood half-lives of liposomes. These findings were expounded upon by Gabizon et al. (Proc. Natl. Acad. Sci. U.S.A., (1988), 85:6949). U.S. Pat. No. 4,837,028 and WO 88/04924, both to Allen et al., disclose liposomes including (1) sphingomyelin and (2) the ganglioside G_{M1} or a galactocerebroside sulfate ester. U.S. Pat. No. 5,543,152 (Webb et al.) discloses liposomes including sphingomyelin. Liposomes including 1,2-sn-dimyristoylphosphatidylcholine are disclosed in WO 97/13499 (Lim et al).

In one embodiment, cationic liposomes are used. Cationic liposomes possess the advantage of being able to fuse to the cell membrane. Non-cationic liposomes, although not able to fuse as efficiently with the plasma membrane, are taken up by macrophages in vivo and can be used to deliver oligonucleotides to macrophages.

Further advantages of liposomes include: liposomes obtained from natural phospholipids are biocompatible and biodegradable; liposomes can incorporate a wide range of water and lipid soluble drugs; liposomes can protect encapsulated oligonucleotides in their internal compartments from metabolism and degradation (Rosoff, in “Pharmaceutical Dosage Forms,” Lieberman, Rieger and Banker (Eds.), 1988, volume 1, p. 245). Important considerations in the preparation of liposome formulations are the lipid surface charge, vesicle size and the aqueous volume of the liposomes.

A positively charged synthetic cationic lipid, N-[1-(2,3-dioleyloxy)propyl]-N,N,N-trimethylammonium chloride (DOTMA) can be used to form small liposomes that interact spontaneously with nucleic acid to form lipid-nucleic acid complexes which are capable of fusing with the negatively charged lipids of the cell membranes of tissue culture cells, resulting in delivery of oligonucleotides (see, e.g., Feigner, P. L. et al., (1987) Proc. Natl. Acad. Sci. USA 8:7413-7417, and U.S. Pat. No. 4,897,355 for a description of DOTMA and its use with DNA).

A DOTMA analogue, 1,2-bis(oleoyloxy)-3-(trimethylammonia)propane (DOTAP) can be used in combination with a phospholipid to form DNA-complexing vesicles. LIPOFECTIN™ Bethesda Research Laboratories, Gaithersburg, Md.) is an effective agent for the delivery of highly anionic nucleic acids into living tissue culture cells that include positively charged DOTMA liposomes which interact spontaneously with negatively charged polynucleotides to form complexes. When enough positively charged liposomes are used, the net charge on the resulting complexes is also positive. Positively charged complexes prepared in this way spontaneously attach to negatively charged cell surfaces, fuse with the plasma membrane, and efficiently deliver functional nucleic acids into, for example, tissue culture cells. Another commercially available cationic lipid, 1,2-bis(oleoyloxy)-3,3-(trimethylammonia)propane (“DOTAP”) (Boehringer Mannheim, Indianapolis, id.) differs from DOTMA in that the oleoyl moieties are linked by ester, rather than ether linkages.

Other reported cationic lipid compounds include those that have been conjugated to a variety of moieties including, for example, carboxyspermine which has been conjugated to one of two types of lipids and includes compounds such as 5-carboxyspermylglycine dioctaoleoylamide (“DOGS”) (TRANSFECTAM™, Promega, Madison, Wis.) and dipalmitoylphosphatidylethanolamine 5-carboxyspermyl-amide (“DPPES”) (see, e.g., U.S. Pat. No. 5,171,678).

Another cationic lipid conjugate includes derivatization of the lipid with cholesterol (“DC-Chol”) which has been formulated into liposomes in combination with DOPE (See, Gao, X. and Huang, L., (1991) Biochim. Biophys. Res. Commun. 179:280). Lipopolylysine, made by conjugating polylysine to DOPE, has been reported to be effective for transfection in the presence of serum (Zhou, X. et al., (1991) Biochim. Biophys. Acta 1065:8). For certain cell lines, these liposomes containing conjugated cationic lipids, are said to exhibit lower toxicity and provide more efficient transfection than the DOTMA-containing compositions. Other commercially available cationic lipid products include DMRIE and DMRIE-HP (Vical, La Jolla, Calif.) and Lipofectamine (DOSPA) (Life Technology, Inc., Gaithersburg, Md.). Other cationic lipids suitable for the delivery of oligonucleotides are described in WO 98/39359 and WO 96/37194.

Liposomal formulations are particularly suited for topical administration, liposomes present several advantages over other formulations. Such advantages include reduced side effects related to high systemic absorption of the administered drug, increased accumulation of the administered drug at the desired target, and the ability to administer oligonucleotides into the skin. In some implementations, liposomes are used for delivering oligonucleotides to epidermal cells and also to enhance the penetration of oligonucleotides into dermal tissues, e.g., into skin. For example, the liposomes can be applied topically. Topical delivery of drugs formulated as liposomes to the skin has been documented (see, e.g., Weiner et al., (1992) Journal of Drug Targeting, vol. 2, 405-410 and du Plessis et al., (1992) Antiviral Research, 18:259-265; Mannino, R. J. and Fould-Fogerite, S., (1998) Biotechniques 6:682-690; Itani, T. et al., (1987) Gene 56:267-276; Nicolau, C. et al. (1987) Meth. Enzymol. 149:157-176; Straubinger, R. M. and Papahadjopoulos, D. (1983) Meth. Enzymol. 101:512-527; Wang, C. Y. and Huang, L., (1987) Proc. Natl. Acad. Sci. USA 84:7851-7855).

Non-ionic liposomal systems have also been examined to determine their utility in the delivery of drugs to the skin, in particular systems including non-ionic surfactant and cholesterol. Non-ionic liposomal formulations including Novasome I (glyceryl dilaurate/cholesterol/polyoxyethylene-10-stearyl ether) and Novasome II (glyceryl distearate/cholesterol/polyoxyethylene-10-stearyl ether) were used to deliver a drug into the dermis of mouse skin. Such formulations with oligonucleotide are useful for treating a dermatological disorder.

The targeting of liposomes is also possible based on, for example, organ-specificity, cell-specificity, and organelle-specificity and is known in the art. In the case of a liposomal targeted delivery system, lipid groups can be incorporated into the lipid bilayer of the liposome in order to maintain the targeting ligand in stable association with the liposomal bilayer. Various linking groups can be used for joining the lipid chains to the targeting ligand. Additional methods are known in the art and are described, for example in U.S. Patent Application Publication No. 20060058255, the linking groups of which are herein incorporated by reference.

Liposomes that include oligonucleotides can be made highly deformable. Such deformability can enable the liposomes to penetrate through pore that are smaller than the average radius of the liposome. For example, transfersomes are yet another type of liposomes, and are highly deformable lipid aggregates which are attractive candidates for drug delivery vehicles. Transfersomes can be described as lipid droplets which are so highly deformable that they are easily able to penetrate through pores which are smaller than the droplet. Transfersomes can be made by adding surface edge activators, usually surfactants, to a standard liposomal composition. Transfersomes that include oligonucleotides can be delivered, for example, subcutaneously by infection in order to deliver oligonucleotides to keratinocytes in the skin. In order to cross intact mammalian skin, lipid vesicles must pass through a series of fine pores, each with a diameter less than 50 nm, under the influence of a suitable transdermal gradient. In addition, due to the lipid properties, these transfersomes can be self-optimizing (adaptive to the shape of pores, e.g., in the skin), self-repairing, and can frequently reach their targets without fragmenting, and often self-loading. Transfersomes have been used to deliver serum albumin to the skin. The transfersome-mediated delivery of serum albumin has been shown to be as effective as subcutaneous injection of a solution containing serum albumin.

Other formulations amenable to the present invention are described in U.S. provisional application Ser. No. 61/018,616, filed Jan. 2, 2008; 61/018,611, filed Jan. 2, 2008; 61/039,748, filed Mar. 26, 2008; 61/047,087, filed Apr. 22, 2008 and 61/051,528, filed May 8, 2008. PCT application No. PCT/US2007/080331, filed Oct. 3, 2007 also describes formulations that are amenable to the present invention.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285).

If the surfactant molecule is not ionized, it is classified as a nonionic surfactant. Nonionic surfactants find wide application in pharmaceutical and cosmetic products and are usable over a wide range of pH values. In general, their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285).

The oligonucleotide for use in the methods of the invention can also be provided as micellar formulations. Micelles are a particular type of molecular assembly in which amphipathic molecules are arranged in a spherical structure such that all the hydrophobic portions of the molecules are directed inward, leaving the hydrophilic portions in contact with the surrounding aqueous phase. The converse arrangement exists if the environment is hydrophobic.

ii. Lipid Nanoparticle-Based Delivery Methods

Oligonucleotides for use in the methods of in the invention may be fully encapsulated in a lipid formulation, e.g., a lipid nanoparticle (LNP), or other nucleic acid-lipid particles. LNPs are extremely useful for systemic applications, as they exhibit extended circulation lifetimes following intravenous (i.v.) injection and accumulate at distal sites (e.g., sites physically separated from the administration site). LNPs include “pSPLP,” which include an encapsulated condensing agent-nucleic acid complex as set forth in PCT Publication No. WO 00/03683. The particles of the present invention typically have a mean diameter of about 50 nm to about 150 nm, more typically about 60 nm to about 130 nm, more typically about 70 nm to about 110 nm, most typically about 70 nm to about 90 nm, and are substantially nontoxic. In addition, the nucleic acids when present in the nucleic acid-lipid particles of the present invention are resistant in aqueous solution to degradation with a nuclease. Nucleic acid-lipid particles and their method of preparation are disclosed in, e.g., U.S. Pat. Nos. 5,976,567; 5,981,501; 6,534,484; 6,586,410; 6,815,432; U.S. Publication No. 2010/0324120 and PCT Publication No. WO 96/40964.

In one embodiment, the lipid to drug ratio (mass/mass ratio) (e.g., lipid to oligonucleotide ratio) will be in the range of from about 1:1 to about 50:1, from about 1:1 to about 25:1, from about 3:1 to about 15:1, from about 4:1 to about 10:1, from about 5:1 to about 9:1, or about 6:1 to about 9:1. Ranges intermediate to the above recited ranges are also contemplated to be part of the invention.

Non-limiting examples of cationic lipid include N,N-dioleyl-N,N-dimethylammonium chloride (DODAC), N,N-distearyl-N,N-dimethylammonium bromide (DDAB), N-(I-(2,3-dioleoyloxy)propyl)-N,N,N-trimethylammonium chloride (DOTAP), N-(I-(2,3-dioleyloxy)propyl)-N,N,N-trimethylammonium chloride (DOTMA), N,N-dimethyl-2,3-dioleyloxy)propylamine (DODMA), 1,2-DiLinoleyloxy-N,N-dimethylaminopropane (DLinDMA), 1,2-Dilinolenyloxy-N,N-dimethylaminopropane (DLenDMA), 1,2-Dilinoleylcarbamoyloxy-3-dimethylaminopropane (DLin-C-DAP), 1,2-Dilinoleyoxy-3-(dimethylamino)acetoxypropane (DLin-DAC), 1,2-Dilinoleyoxy-3-morpholinopropane (DLin-MA), 1,2-Dilinoleoyl-3-dimethylaminopropane (DLinDAP), 1,2-Dilinoleylthio-3-dimethylaminopropane (DLin-S-DMA), 1-Linoleoyl-2-linoleyloxy-3-dimethylaminopropane (DLin-2-DMAP), 1,2-Dilinoleyloxy-3-trimethylaminopropane chloride salt (DLin-TMA.Cl), 1,2-Dilinoleoyl-3-trimethylaminopropane chloride salt (DLin-TAP.Cl), 1,2-Dilinoleyloxy-3-(N-methylpiperazino)propane (DLin-MPZ), or 3-(N,N-Dilinoleylamino)-1,2-propanediol (DLinAP), 3-(N,N-Dioleylamino)-1,2-propanedio (DOAP), 1,2-Dilinoleyloxo-3-(2-N,N-dimethylamino)ethoxypropane (DLin-EG-DMA), 1,2-Dilinolenyloxy-N,N-dimethylaminopropane (DLinDMA), 2,2-Dilinoleyl-4-dimethylaminomethyl-[1,3]-dioxolane (DLin-K-DMA) or analogs thereof, (3aR,5s,6aS)-N,N-dimethyl-2,2-di((9Z,12Z)-octadeca-9,12-dienyetetrahydro-3aH-cyclopenta[d][1,3]dioxol-5-amine (ALN100), (6Z,9Z,28Z,31Z)-heptatriaconta-6,9,28,31-tetraen-19-yl4-(dimethylamino)bu-tanoate (MC3), 1,1′-(2-(4-(2-((2-(bis(2-hydroxydodecyl)amino)ethyl)(2-hydroxydodecyl)ami-no)ethyl)piperazin-1-yeethylazanediyedidodecan-2-ol (Tech G1), or a mixture thereof. The cationic lipid can include, for example, from about 20 mol % to about 50 mol % or about 40 mol % of the total lipid present in the particle.

The ionizable/non-cationic lipid can be an anionic lipid or a neutral lipid including, but not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, or a mixture thereof. The non-cationic lipid can be, for example, from about 5 mol % to about 90 mol %, about 10 mol %, or about 58 mol % if cholesterol is included, of the total lipid present in the particle.

The conjugated lipid that inhibits aggregation of particles can be, for example, a polyethyleneglycol (PEG)-lipid including, without limitation, a PEG-diacylglycerol (DAG), a PEG-dialkyloxypropyl (DAA), a PEG-phospholipid, a PEG-ceramide (Cer), or a mixture thereof. The PEG-DAA conjugate can be, for example, a PEG-dilauryloxypropyl (Ci₂), a PEG-dimyristyloxypropyl (Ci₄), a PEG-dipalmityloxypropyl (Ci₆), or a PEG-distearyloxypropyl (C]₈). The conjugated lipid that prevents aggregation of particles can be, for example, from 0 mol % to about 20 mol % or about 2 mol % of the total lipid present in the particle.

In some embodiments, the nucleic acid-lipid particle further includes cholesterol at, e.g., about 10 mol % to about 60 mol % or about 50 mol % of the total lipid present in the particle.

B. Combination Therapies

A method of the invention can be used alone or in combination with an additional therapeutic agent, e.g., other agents that treat the same disorder, e.g., Rett syndrome, or symptoms associated therewith, or in combination with other types of therapies to the disorder. In combination treatments, the dosages of one or more of the therapeutic compounds may be reduced from standard dosages when administered alone. For example, doses may be determined empirically from drug combinations and permutations or may be deduced by isobolographic analysis. Dosages of the compounds when combined should provide a therapeutic effect.

In some embodiments, the second therapeutic agent is medication to address abnormal heart rhythm, or medication to control seizures, e.g., triheptanoin, a mitochondria activity boosting drug, ketamine, an NMDA receptor antagonist, sarizotan, a serotonin/dopamine receptor agonist, or cannabidiol.

The second agent may also be a therapeutic agent which is a non-drug treatment. For example, the second agent may be physical therapy, or speech therapy.]

In any of the combination embodiments described herein, the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

### III. Compositions for Use in the Methods of the Invention

The compositions for use in the methods of the present invention, i.e., methods of editing a MECP2 polynucleotide, e.g., a MECP2 polynucleotide comprising a single nucleotide polymorphism (SNP) associated with Rett syndrome, and methods for treating or preventing a MECP2-associated disease, e.g., Rett syndrome, in a subject, include a guide oligonucleotide capable of effecting an adenosine deaminase acting on RNA (ADAR)-mediated adenosine to inosine alteration of the SNP associated with Rett syndrome.

The oligonucleotides, or guide oligonucleotides, for use in the methods of the invention may be utilized to deaminate target adenosines on a specific mRNA, e.g., an adenosine which may be deaminated to produce a therapeutic result, e.g., in a subject in need thereof.

Examples of modifications resulting from deamination of target adenosines within a target codon are provided in Tables 1 and 2 below.

TABLE 1

| Target | Amino Acid Encoded | Modified | Amino Acid Encoded |
|---|---|---|---|
| Codon | by Target Codon | Codon | by Modified Codon |
| AAA | Lys | IAA | Glu |
| | | AIA | Arg |
| | | IIA | Gly |
| | | AII | Arg |
| | | IAI | Glu |
| | | III | Gly |
| AAC | Asn | IAC | Asp |
| | | AIC | Ser |
| | | IIC | Gly |
| AAG | Lys | IAG | Glu |
| | | AIG | Arg |
| | | IIG | Gly |
| AAU | Arg | IAU | Asp |
| | | AIU | Ser |
| | | IIU | Gly |
| ACA | Thr | ICA | Ala |
| | | ICI | Ala |
| ACC | Thr | ICC | Ala |
| ACG | Thr | ICG | Ala |
| ACU | Thr | ICU | Ala |
| AGA | Arg | IGA | Gly |
| | | IGI | Gly |
| AGC | Ser | IGC | Gly |
| AGG | Arg | IGG | Gly |
| AGU | Ser | IGU | Gly |
| AUA | Ile | IUA | Asp |
| | | AUI | Met |
| | | IUI | Val |
| AUC | Ile | IUC | Val |
| AUG | Met | IUG | Val |
| AUU | Ile | IUU | Val |
| CAA | Gln | CIA | Arg |
| | | CII | Arg |
| CAC | His | CIC | Arg |
| CAG | Gln | CIG | Arg |
| CAU | His | CIU | Arg |
| GAA | Glu | GIA | Gly |
| | | GII | Gly |
| GAC | Asp | GIC | Gly |
| GAG | Glu | GIG | Gly |
| GAU | Asp | GIU | Gly |
| UAA | Stop | UII | Trp |
| UGA | Stop | UGI | Trp |
| UAC | Tyr | UIC | Cys |
| UAG | Stop | UIG | Trp |
| UAU | Tyr | UIU | Cys |

TABLE 2
Target Codon Base Composition and Resulting Modified Codon

| Target Codon | Modified Codon |
|---|---|
| AAA | AIA |
| AAC | AIC |
| AAG | AIG |
| AAU | AIU |
| CAA | CIA |
| CAC | CIC |
| CAG | CIG |
| CAU | CIU |
| GAA | GIA |
| GAC | GIC |
| GAG | GIG |
| GAU | GIU |
| UAA | UIA |
| UAC | UIC |
| UAG | UIG |
| UAU | UIU |

Because the deamination of the adenosine to an inosine may result in a protein that no longer bears the mutated A at the target position, the identification of the deamination into inosine may be a functional read-out, for instance an assessment on whether a functional protein is present, or even the assessment that a disease that is caused by the presence of the adenosine is (partly) reversed. The functional assessment for each of the diseases mentioned herein will generally be according to methods known to the skilled person. When the presence of a target adenosine causes aberrant splicing, the read-out may be the assessment of whether the aberrant splicing is still taking place, or not, or less. On the other hand, when the deamination of a target adenosine is wanted to introduce a splice site, then similar approaches can be used to check whether the required type of splicing is indeed taking place. A very suitable manner to identify the presence of an inosine after deamination of the target adenosine is of course RT-PCR and sequencing, using methods that are well-known to the person skilled in the art.

In general, mutations in any target RNA that can be reversed using oligonucleotide constructs according to the invention are G-to-A mutations, and oligonucleotide constructs can be designed accordingly. Mutations that may be targeted using oligonucleotide constructs according to the invention also include C to A, U to A (T to A on the DNA level) in the case of recruiting adenosine deaminases. Although RNA editing in the latter circumstances may not necessarily revert the mutation to wild-type, the edited nucleotide may give rise to an improvement over the original mutation. For example, a mutation that causes an in frame stop codon—giving rise to a truncated protein, upon translation—may be changed into a codon coding for an amino acid that may not be the original amino acid in that position, but that gives rise to a (full length) protein with at least some functionality, at least more functionality than the truncated protein.

### Oligonucleotide Agents

The oligonucleotides for use in the methods of the present invention are complementary to target mRNA sequence, e.g., MECP2, comprising the SNP associated with a disease, e.g., Rett syndrome. In some embodiments, the guide oligonucleotides are complementary to target mRNA with the exception of at least one mismatch. The oligonucleotide includes a mismatch opposite the target adenosine.

The guide oligonucleotides are also capable of recruiting adenosine deaminase acting on RNA (ADAR) enzymes to deaminate selected adenosines on the target mRNA. In some embodiments, the oligonucleotide further comprises one or more ADAR-recruiting domains. In some embodiments, only one adenosine is deaminated. In some embodiments, 1, 2, or 3 adenosines are deaminated.

The oligonucleotides for use in the methods of the invention may further include modifications (e.g., alternative nucleotides) to increase stability and/or increase deamination efficiency.

Whenever reference is made to nucleotides in the guide oligonucleotide, such as cytosine, 5-methylcytosine, 5-hydroxymethylcytosine, Pyrrolocytidine, and -D-Glucosyl-5-hydroxy-methylcytosine are included; when reference is made to adenine, 2-aminopurine, 2,6-diaminopurine, 3-deazaadenosine, 7-deazaadenosine, 8-azidoadenosine, 8-methyladenosine, 7-aminomethyl-7-deazaguanosine, 7-deazaguanosine, N6-Methyladenine and 7-methyladenine are included; when reference is made to uracil, 5-methoxyuracil, 5-methyluracil, dihydrouracil, pseudouracil, and thienouracil, dihydrouracil, 4-thiouracil and 5-hydroxymethyluracil are included; when reference is made to guanosine, 7-methylguanosine, 8-aza-7-deazaguanosine, thienoguanosine and 1-methylguanosine are included.

Whenever reference is made to nucleosides or nucleotides, ribofuranose derivatives, such as 2′-deoxy, 2′-hydroxy, 2-fluororibose and 2′-O-substituted variants, such as 2′-O-methyl, are included, as well as other modifications, including 2′-4′ bridged variants.

Whenever reference is made to oligonucleotides, linkages between two mono-nucleotides may be phosphodiester linkages as well as modifications thereof, including, phosphodiester, phosphotriester, phosphoro(di)thioate, methylphosphonate, phosphor-amidate linkers, and the like.

### Modifications

A guide oligonucleotide according to the present invention may be chemically modified almost in its entirety, for example by modifying all nucleotides with a 2′-O-methylated sugar moiety (2′-OMe). Various chemistries and modifications are known in the field of oligonucleotides that can be readily used in accordance with the invention. The regular internucleosidic linkages between the nucleotides may be altered by mono- or di-thioation of the phosphodiester bonds to yield phosphorothioate esters or phosphorodithioate esters, respectively.

Other modifications of the internucleosidic linkages are possible, including amidation and peptide linkers. In some embodiments, the guide oligonucleotides of the present invention have one, two, three, four or more phosphorothioate linkages. It will be understood by the skilled person that the number of such linkages may vary on each end, depending on the target sequence, or based on other aspects, such as toxicity.

The ribose sugar may be modified by substitution of the 2′-O moiety with a lower alkyl (C1-4, such as 2′-O-methyl), alkenyl (C2-4), alkynyl (C2-4), methoxyethyl (2′-O-MOE), —H (as in DNA) or other substituent. Preferred substituents of the 2′-OH group are a methyl, methoxyethyl or 3,3′-dimethylallyl group. The latter is known for its property to inhibit nuclease sensitivity due to its bulkiness, while improving efficiency of hybridization (Angus & Sproat. 1993. FEBS Vol. 325, no. 1, 2, 123-7). Alternatively, locked nucleic acid sequences (LNAs), comprising a 2′-4′ intramolecular bridge (usually a methylene bridge between the 2′ oxygen and 4′ carbon) linkage inside the ribose ring, or 2′-fluoroarabinonucleosides (FANA), may be applied. Purine nucleobases and/or pyrimidine nucleobases may be modified to alter their properties, for example by amination or deamination of the heterocyclic rings. The exact chemistries and formats may vary from oligonucleotide construct to oligonucleotide construct and from application to application. It is believed that 4 or more consecutive DNA nucleotides (4 consecutive deoxyriboses) in an oligonucleotide create so-called gapmers that—when annealed to their RNA cognate sequences—induce cleavage of the target RNA by RNaseH. According to the present invention, RNaseH cleavage of the target RNA is generally to be avoided as much as possible.

Examples of chemical modifications in the guide oligonucleotides of the present invention are modifications of the sugar moiety, including by cross-linking substituents within the sugar (ribose) moiety (e.g., as in locked nucleic acids: LNA), by substitution of the 2′-O atom with alkyl (e.g. 2′-O-methyl), alkynyl (2′-O-alkynyl), alkenyl (2′-O-alkenyl), alkoxyalkyl (e.g. methoxyethyl: 2′-O-MOE) groups, having a length as specified above, and the like. In addition, the phosphodiester group of the backbone may be modified by thioation, dithioation, amidation and the like to yield phosphorothioate, phosphorodithioate, phosphoramidate, etc., internucleosidic linkages. The internucleotidic linkages may be replaced in full or in part by peptidic linkages to yield in peptidonucleic acid sequences and the like. Alternatively, or in addition, the nucleobases may be modified by (de)amination, to yield inosine or 2′6′-diaminopurines and the like. A further modification may be methylation of the C5 in the cytidine moiety of the nucleotide, to reduce potential immunogenic properties known to be associated with CpG sequences

The inventors of the present invention surprisingly discovered that relative to the editing observed for a guide oligonucleotide that is fully 2′-OMe modified with a DNA triplet, generally editing is higher for guide oligonucleotides comprising a FANA triplet.

### Mismatches

The inventors of the present invention have discovered that mismatches, wobbles and/or out-looping bulges (caused by nucleotides in the guide oligonucleotide that do not form perfect base pairs with the target RNA according to the Watson-Crick base pairing rules) are generally tolerated and may improve editing activity of the target RNA sequence. The number of mismatches, wobbles or bulges in the guide oligonucleotide of the present invention (when it hybridizes to its RNA target sequence) may be one (which may be the one mismatch formed at the target adenosine position, when a cytosine is the opposite nucleoside, or some other position in the guide oligonucleotide) or more (either including or not including the mismatch at the target adenosine), depending on the length of the guide oligonucleotide. Additional mismatches, wobbles or bulges may be upstream as well as downstream of the target adenosine. In some embodiments, a mismatch or wobble is present at position 12 nucleotides upstream (towards the 5′ end) from the targeted adenosine. In some embodiments, a mismatch or wobble is present at position 16 nucleotides upstream (towards the 5′ end) from the targeted adenosine. In some embodiments, a mismatch or wobble is present at position 17 nucleotides upstream (towards the 5′ end) from the targeted adenosine. In some embodiments, a mismatch or wobble is present at position 21 nucleotides upstream (towards the 5′ end) from the targeted adenosine. The bulges or mismatches may be at a single position (caused by one mismatching, wobble or bulge base pair) or a series of nucleotides that are not fully complementary (caused by more than one consecutive mismatching or wobble base pair or bulge, preferably two or three consecutive mismatching and/or wobble base pairs and/or bulges).

A. Alternative Oligonucleotides

In one embodiment, one or more of the nucleotides of the oligonucleotide of the invention, is naturally-occurring, and does not include, e.g., chemical modifications and/or conjugations known in the art and described herein. In another embodiment, one or more of the nucleotides of an oligonucleotide of the invention, is chemically modified to enhance stability or other beneficial characteristics (e.g., alternative nucleotides). Without being bound by theory, it is believed that certain modification can increase nuclease resistance and/or serum stability, or decrease immunogenicity. For example, polynucleotides of the invention may contain nucleotides found to occur naturally in DNA or RNA (e.g., adenine, thymidine, guanosine, cytidine, uridine, or inosine) or may contain nucleotides which have one or more chemical modifications to one or more components of the nucleotide (e.g., the nucleobase, sugar, or phospho-linker moiety). Oligonucleotides of the invention may be linked to one another through naturally-occurring phosphodiester bonds, or may be modified to be covalently linked through phosphorothiorate, 3′-methylenephosphonate, 5′-methylenephosphonate, 3′-phosphoamidate, 2′-5′ phosphodiester, guanidinium, S-methylthiourea, or peptide bonds.

In some embodiments, one or more of the nucleotides of the oligonucleotide of the invention has the structure of any one of Formula I-V:

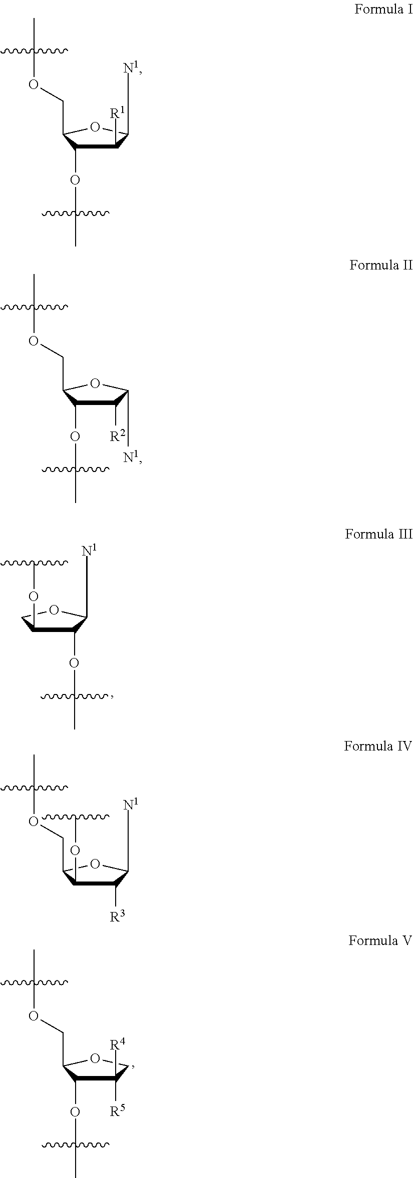

In some embodiments, one or more of the nucleotides of the oligonucleotide of the invention has the structure of any one of Formula I, e.g., has the structure:

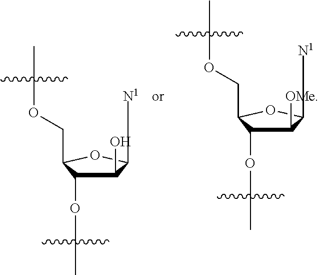

In some embodiments, one or more of the nucleotides of the oligonucleotide of the invention has the structure of any one of Formula II, e.g., has the structure:

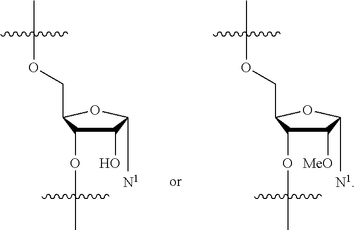

In some embodiments, one or more of the nucleotides of the oligonucleotide of the invention has the structure of any one of Formula III.

In some embodiments, one or more of the nucleotides of the oligonucleotide of the invention has the structure of any one of Formula IV, e.g., has the structure:

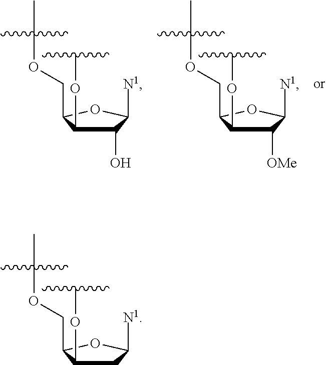

In some embodiments, one or more of the nucleotides of the oligonucleotide of the invention has the structure of any one of Formula V, e.g., has the structure:

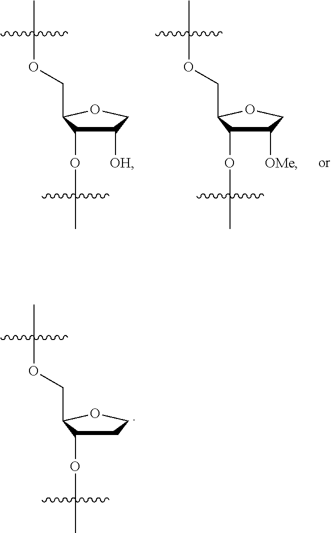

In certain embodiments of the invention, substantially all of the nucleotides of an oligonucleotide of the invention are alternative nucleotides. In other embodiments of the invention, all of the nucleotides of an oligonucleotide of the invention are alternative nucleotides. Oligonucleotides of the invention in which “substantially all of the nucleotides are alternative nucleotides” are largely but not wholly modified and can include no more than 5, 4, 3, 2, or 1 naturally-occurring nucleotides. In still other embodiments of the invention, oligonucleotides of the invention can include no more than 5, 4, 3, 2, or 1 alternative nucleotides.

In some embodiments, the oligonucleotides of the instant invention include the structure:

[Aₘ]-X¹-X²-X³—[Bₙ]

wherein each of A and B is a nucleotide; m and n are each, independently, an integer from 5 to 40; at least one of X¹, X², and X³ has the structure of Formula I, wherein R¹ is fluoro, hydroxy, or methoxy and N¹ is a nucleobase, or the structure of Formula V, wherein R⁴ is hydrogen and R⁵ is hydrogen; each of X¹, X², and X³ that does not have the structure of Formula I is a ribonucleotide; [Aₘ] and [Bₙ] each include at least five terminal 2′-O-methyl-nucleotides; at least four terminal phosphorothioate linkages, and at least 20% of the nucleotides of [Aₘ] and [Bₙ] combined are 2′-O-methyl-nucleotides. In some embodiments, X¹ includes an adenine nucleobase, X² includes a cytosine, 5-methylcytosine, uracil, or thymine nucleobase or does not include a nucleobase, and X³ includes an adenine nucleobase; X¹ includes an adenine nucleobase, X² includes a cytosine, 5-methylcytosine, uracil, or thymine nucleobase or does not include a nucleobase, and X³ includes a guanine or hypoxanthine nucleobase; X¹ includes an adenine nucleobase, X² includes a cytosine, 5-methylcytosine, uracil, or thymine nucleobase or does not include a nucleobase, and X³ includes a uracil or thymine nucleobase; X¹ includes an adenine nucleobase, X² includes a cytosine, 5-methylcytosine, uracil, or thymine nucleobase or does not include a nucleobase, and X³ includes a cytosine or 5-methylcytosine nucleobase; X¹ includes a guanine or hypoxanthine nucleobase, X² includes a cytosine, 5-methylcytosine, uracil, or thymine nucleobase or does not include a nucleobase, and X³ includes an adenine nucleobase; X¹ includes a guanine or hypoxanthine nucleobase, X² includes a cytosine, 5-methylcytosine, uracil, or thymine nucleobase or does not include a nucleobase, and X³ includes a guanine or hypoxanthine nucleobase; X¹ includes a guanine or hypoxanthine nucleobase, X² includes a cytosine, 5-methylcytosine, uracil, or thymine nucleobase or does not include a nucleobase, and X³ includes a uracil or thymine nucleobase; X¹ includes a guanine or hypoxanthine nucleobase, X² includes a cytosine, 5-methylcytosine, uracil, or thymine nucleobase or does not include a nucleobase, and X³ includes a cytosine or 5-methylcytosine nucleobase; X¹ includes a uracil or thymine nucleobase, X² includes a cytosine, 5-methylcytosine, uracil, or thymine nucleobase or does not include a nucleobase, and X³ includes an adenine nucleobase; X¹ includes a uracil or thymine nucleobase, X² includes a cytosine, 5-methylcytosine, uracil, or thymine nucleobase or does not include a nucleobase, and X³ includes a guanine or hypoxanthine nucleobase; X¹ includes a uracil or thymine nucleobase, X² includes a cytosine, 5-methylcytosine, uracil, or thymine nucleobase or does not include a nucleobase, and X³ includes a uracil or thymine nucleobase; X¹ includes a uracil or thymine nucleobase, X² includes a cytosine, 5-methylcytosine, uracil, or thymine nucleobase or does not include a nucleobase, and X³ includes a cytosine or 5-methylcytosine nucleobase; X¹ includes a cytosine or 5-methylcytosine nucleobase, X² includes a cytosine, 5-methylcytosine, uracil, or thymine nucleobase or does not include a nucleobase, and X³ includes an adenine nucleobase; X¹ includes a cytosine or 5-methylcytosine nucleobase, X² includes a cytosine, 5-methylcytosine, uracil, or thymine nucleobase or does not include a nucleobase, and X³ includes a guanine or hypoxanthine nucleobase; X¹ includes a cytosine or 5-methylcytosine nucleobase, X² includes a cytosine, 5-methylcytosine, uracil, or thymine nucleobase or does not include a nucleobase, and X³ includes a uracil or thymine nucleobase; or X¹ includes a cytosine or 5-methylcytosine nucleobase, X² includes a cytosine, 5-methylcytosine, uracil, or thymine nucleobase or does not include a nucleobase, and X³ includes a cytosine or 5-methylcytosine nucleobase.

In some embodiments, one or more of the nucleotides of the oligonucleotide of the invention has the structure of any one of Formula VI-XI:

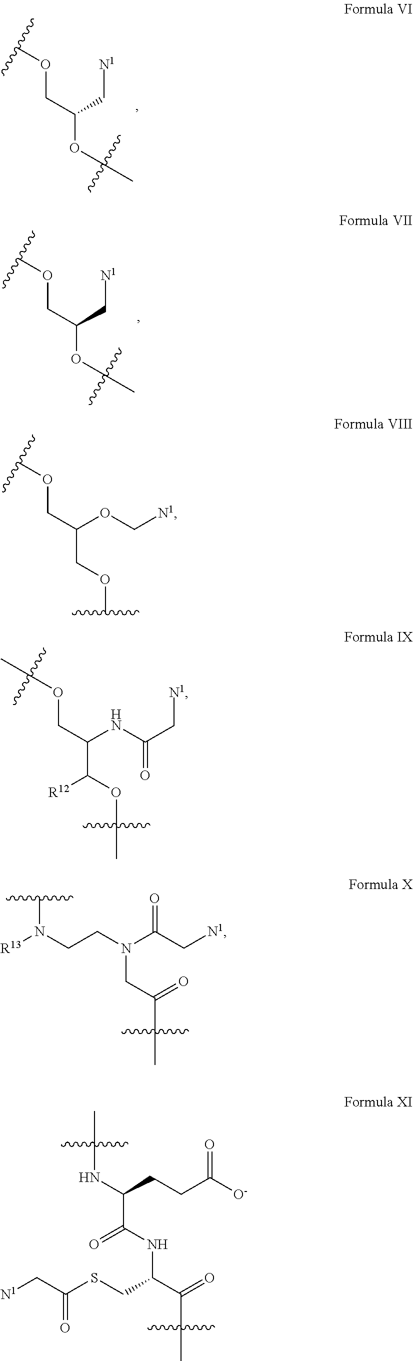

In some embodiments, one or more of the nucleotides of the oligonucleotide of the invention has the structure of any one of Formula VI.

In some embodiments, one or more of the nucleotides of the oligonucleotide of the invention has the structure of any one of Formula VII.

In some embodiments, one or more of the nucleotides of the oligonucleotide of the invention has the structure of any one of Formula VIII.

In some embodiments, one or more of the nucleotides of the oligonucleotide of the invention has the structure of any one of Formula IX, e.g., has the structure:

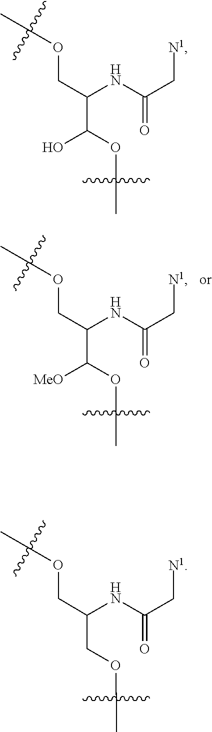

In some embodiments, one or more of the nucleotides of the oligonucleotide of the invention has the structure of any one of Formula X, e.g., has the structure:

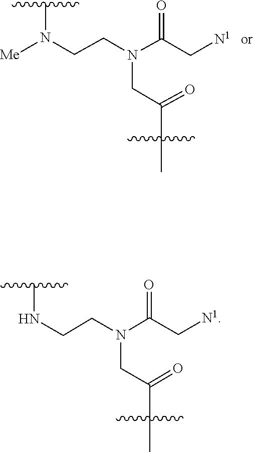

In some embodiments, one or more of the nucleotides of the oligonucleotide of the invention has the structure of any one of Formula XI, e.g., has the structure:

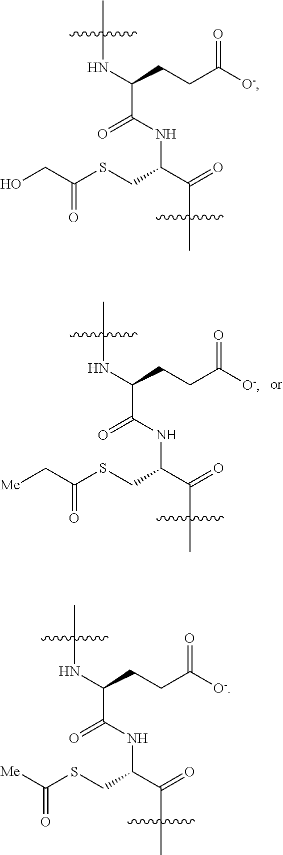

In certain embodiments of the invention, substantially all of the nucleotides of an oligonucleotide of the invention are alternative nucleotides. In other embodiments of the invention, all of the nucleotides of an oligonucleotide of the invention are alternative nucleotides. Oligonucleotides of the invention in which “substantially all of the nucleotides are alternative nucleotides” are largely but not wholly modified and can include no more than 5, 4, 3, 2, or 1 naturally-occurring nucleotides. In still other embodiments of the invention, oligonucleotides of the invention can include no more than 5, 4, 3, 2, or 1 alternative nucleotides.

In some embodiments of the invention, the oligonucleotides of the instant invention include the structure:

[Aₘ]-X¹-X²-X³—[Bₙ]

wherein each of A and B is a nucleotide; m and n are each, independently, an integer from 5 to 40; at least one of X¹, X², and X³ has the structure of Formula VI, Formula VII, Formula VIII, or Formula IX, wherein N¹ is a nucleobase and each of X¹, X², and X³ that does not have the structure of Formula VI, Formula VII, Formula VIII, or Formula IX is a ribonucleotide; [Aₘ] and [Bₙ] each include at least five terminal 2′-O-methyl-nucleotides and at least four terminal phosphorothioate linkages; and at least 20% of the nucleotides of [Aₘ] and [Bₙ] combined are 2′-O-methyl-nucleotides. In some embodiments, X¹ includes an adenine nucleobase, X² includes a cytosine, 5-methylcytosine, uracil, or thymine nucleobase or does not include a nucleobase, and X³ includes an adenine nucleobase; X¹ includes an adenine nucleobase, X² includes a cytosine, 5-methylcytosine, uracil, or thymine nucleobase or does not include a nucleobase, and X³ includes a guanine or hypoxanthine nucleobase; X¹ includes an adenine nucleobase, X² includes a cytosine, 5-methylcytosine, uracil, or thymine nucleobase or does not include a nucleobase, and X³ includes a uracil or thymine nucleobase; X¹ includes an adenine nucleobase, X² includes a cytosine, 5-methylcytosine, uracil, or thymine nucleobase or does not include a nucleobase, and X³ includes a cytosine or 5-methylcytosine nucleobase; X¹ includes a guanine or hypoxanthine nucleobase, X² includes a cytosine, 5-methylcytosine, uracil, or thymine nucleobase or does not include a nucleobase, and X³ includes an adenine nucleobase; X¹ includes a guanine or hypoxanthine nucleobase, X² includes a cytosine, 5-methylcytosine, uracil, or thymine nucleobase or does not include a nucleobase, and X³ includes a guanine or hypoxanthine nucleobase; X¹ includes a guanine or hypoxanthine nucleobase, X² includes a cytosine, 5-methylcytosine, uracil, or thymine nucleobase or does not include a nucleobase, and X³ includes a uracil or thymine nucleobase; X¹ includes a guanine or hypoxanthine nucleobase, X² includes a cytosine, 5-methylcytosine, uracil, or thymine nucleobase or does not include a nucleobase, and X³ includes a cytosine or 5-methylcytosine nucleobase; X¹ includes a uracil or thymine nucleobase, X² includes a cytosine, 5-methylcytosine, uracil, or thymine nucleobase or does not include a nucleobase, and X³ includes an adenine nucleobase; X¹ includes a uracil or thymine nucleobase, X² includes a cytosine, 5-methylcytosine, uracil, or thymine nucleobase or does not include a nucleobase, and X³ includes a guanine or hypoxanthine nucleobase; X¹ includes a uracil or thymine nucleobase, X² includes a cytosine, 5-methylcytosine, uracil, or thymine nucleobase or does not include a nucleobase, and X³ includes a uracil or thymine nucleobase; X¹ includes a uracil or thymine nucleobase, X² includes a cytosine, 5-methylcytosine, uracil, or thymine nucleobase or does not include a nucleobase, and X³ includes a cytosine or 5-methylcytosine nucleobase; X¹ includes a cytosine or 5-methylcytosine nucleobase, X² includes a cytosine, 5-methylcytosine, uracil, or thymine nucleobase or does not include a nucleobase, and X³ includes an adenine nucleobase; X¹ includes a cytosine or 5-methylcytosine nucleobase, X² includes a cytosine, 5-methylcytosine, uracil, or thymine nucleobase or does not include a nucleobase, and X³ includes a guanine or hypoxanthine nucleobase; X¹ includes a cytosine or 5-methylcytosine nucleobase, X² includes a cytosine, 5-methylcytosine, uracil, or thymine nucleobase or does not include a nucleobase, and X³ includes a uracil or thymine nucleobase; or X¹ includes a cytosine or 5-methylcytosine nucleobase, X² includes a cytosine, 5-methylcytosine, uracil, or thymine nucleobase or does not include a nucleobase, and X³ includes a cytosine or 5-methylcytosine nucleobase.

In some embodiments, one or more of the nucleotides of the oligonucleotide of the invention has the structure of any one of Formula XII-XV:

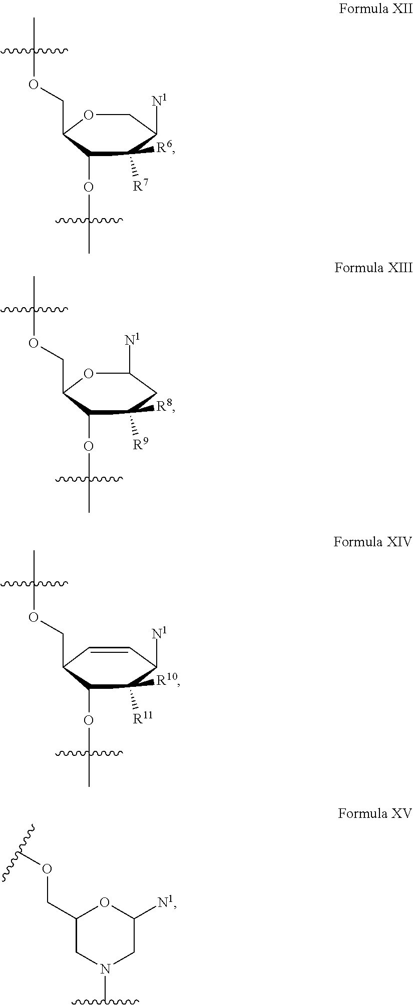

In some embodiments, one or more of the nucleotides of the oligonucleotide of the invention has the structure of any one of Formula XII, e.g., has the structure:

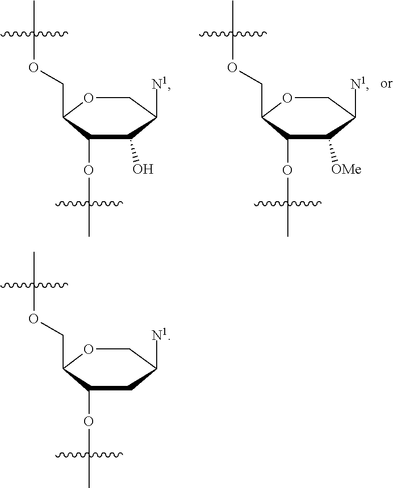

In some embodiments, one or more of the nucleotides of the oligonucleotide of the invention has the structure of any one of Formula XIII, e.g., has the structure:

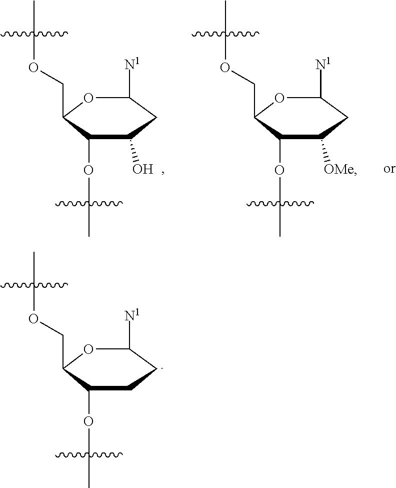

In some embodiments, one or more of the nucleotides of the oligonucleotide of the invention has the structure of any one of Formula XIV, e.g., has the structure:

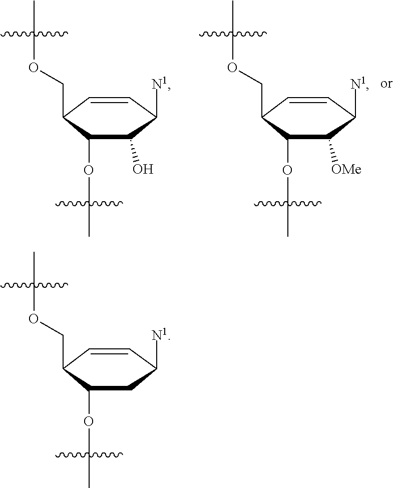

In some embodiments, one or more of the nucleotides of the oligonucleotide of the invention has the structure of any one of Formula XV.

In certain embodiments of the invention, substantially all of the nucleotides of an oligonucleotide of the invention are alternative nucleotides. In other embodiments of the invention, all of the nucleotides of an oligonucleotide of the invention are alternative nucleotides. Oligonucleotides of the invention in which “substantially all of the nucleotides are alternative nucleotides” are largely but not wholly modified and can include no more than 5, 4, 3, 2, or 1 naturally-occurring nucleotides. In still other embodiments of the invention, oligonucleotides of the invention can include no more than 5, 4, 3, 2, or 1 alternative nucleotides.

In some embodiments, the oligonucleotides of the instant invention include the structure:

[Aₘ]-X¹-X²-X³—[Bₙ]

wherein each of A and B is a nucleotide; m and n are each, independently, an integer from 5 to 40; at least of X¹, X², and X³ has the structure of Formula XIII, wherein R⁸ and R⁹ are each hydrogen, and each of X¹, X² and X³ that does not have the structure of Formula XIII is a ribonucleotide; [Aₘ] and [Bₙ] each include at least five terminal 2′-O-methyl-nucleotides and at least four terminal phosphorothioate linkages; and at least 20% of the nucleotides of [Aₘ] and [Bₙ] combined are 2′-O-methyl-nucleotides. In some embodiments, X¹ includes an adenine nucleobase, X² includes a cytosine, 5-methylcytosine, uracil, or thymine nucleobase or does not include a nucleobase, and X³ includes an adenine nucleobase; X¹ includes an adenine nucleobase, X² includes a cytosine, 5-methylcytosine, uracil, or thymine nucleobase or does not include a nucleobase, and X³ includes a guanine or hypoxanthine nucleobase; X¹ includes an adenine nucleobase, X² includes a cytosine, 5-methylcytosine, uracil, or thymine nucleobase or does not include a nucleobase, and X³ includes a uracil or thymine nucleobase; X¹ includes an adenine nucleobase, X² includes a cytosine, 5-methylcytosine, uracil, or thymine nucleobase or does not include a nucleobase, and X³ includes a cytosine or 5-methylcytosine nucleobase; X¹ includes a guanine or hypoxanthine nucleobase, X² includes a cytosine, 5-methylcytosine, uracil, or thymine nucleobase or does not include a nucleobase, and X³ includes an adenine nucleobase; X¹ includes a guanine or hypoxanthine nucleobase, X² includes a cytosine, 5-methylcytosine, uracil, or thymine nucleobase or does not include a nucleobase, and X³ includes a guanine or hypoxanthine nucleobase; X¹ includes a guanine or hypoxanthine nucleobase, X² includes a cytosine, 5-methylcytosine, uracil, or thymine nucleobase or does not include a nucleobase, and X³ includes a uracil or thymine nucleobase; X¹ includes a guanine or hypoxanthine nucleobase, X² includes a cytosine, 5-methylcytosine, uracil, or thymine nucleobase or does not include a nucleobase, and X³ includes a cytosine or 5-methylcytosine nucleobase; X¹ includes a uracil or thymine nucleobase, X² includes a cytosine, 5-methylcytosine, uracil, or thymine nucleobase or does not include a nucleobase, and X³ includes an adenine nucleobase; X¹ includes a uracil or thymine nucleobase, X² includes a cytosine, 5-methylcytosine, uracil, or thymine nucleobase or does not include a nucleobase, and X³ includes a guanine or hypoxanthine nucleobase; X¹ includes a uracil or thymine nucleobase, X² includes a cytosine, 5-methylcytosine, uracil, or thymine nucleobase or does not include a nucleobase, and X³ includes a uracil or thymine nucleobase; X¹ includes a uracil or thymine nucleobase, X² includes a cytosine, 5-methylcytosine, uracil, or thymine nucleobase or does not include a nucleobase, and X³ includes a cytosine or 5-methylcytosine nucleobase; X¹ includes a cytosine or 5-methylcytosine nucleobase, X² includes a cytosine, 5-methylcytosine, uracil, or thymine nucleobase or does not include a nucleobase, and X³ includes an adenine nucleobase; X¹ includes a cytosine or 5-methylcytosine nucleobase, X² includes a cytosine, 5-methylcytosine, uracil, or thymine nucleobase or does not include a nucleobase, and X³ includes a guanine or hypoxanthine nucleobase; X¹ includes a cytosine or 5-methylcytosine nucleobase, X² includes a cytosine, 5-methylcytosine, uracil, or thymine nucleobase or does not include a nucleobase, and X³ includes a uracil or thymine nucleobase; or X¹ includes a cytosine or 5-methylcytosine nucleobase, X² includes a cytosine, 5-methylcytosine, uracil, or thymine nucleobase or does not include a nucleobase, and X³ includes a cytosine or 5-methylcytosine nucleobase.

In some embodiments, the oligonucleotides for use in the methods of the instant invention include a recruitment domain for the ADAR enzyme (e.g., an ADAR-recruiting domain). In some embodiments, the ADAR-recruiting domain is a stem-loop structure. Such oligonucleotides may be referred to as “axiomer AONs” or “self-looping AONs.” The recruitment portion acts in recruiting a natural ADAR enzyme present in the cell to the dsRNA formed by hybridization of the target sequence with the targeting portion. The recruitment portion may be a stem-loop structure mimicking either a natural substrate (e.g. the glutamate ionotropic receptor AMPA type subunit 2 (GluR2) receptor; such as a GluR2 ADAR-recruiting domain) or a Z-DNA structure known to be recognized by the dsRNA binding regions of ADAR enzymes (e.g., a Z-DNA ADAR-recruiting domain). As GluR2 and Z-DNA ADAR-recruiting domains are high affinity binding partners to ADAR, there is no need for conjugated entities or presence of modified recombinant ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion may be a step loop structure described in WO 2016/097212, US 2018/0208924, Merkle et al. Nature Biotechnology, 37: 133-8 (2019), Katrekar et al. Nature Methods, 16(3): 239-42 (2019), Fukuda et al. Scientific Reports, 7: 41478 (2017), the stem-loop structures of the ADAR recruitment portion of which are herein incorporated by reference. In some embodiments, the oligonucleotides include one or more ADAR-recruiting domains (e.g., 1 or 2 ADAR-recruiting domains). In some embodiments, the ADAR-recruiting domain is at the 5′ end of the oligonucleotide. In other embodiments, the ADAR-recruiting domain is at the 3′ end of said oligonucleotide. In some embodiments, the oligonucleotide includes a first ADAR-recruiting domain and a second ADAR-recruiting domain. the first ADAR-recruiting domain is at the 5′ end of said oligonucleotide, and the second ADAR-recruiting domain is at the 3′ end of said oligonucleotide.

In some embodiments, the oligonucleotide includes the structure of Formula XVI:

C-L₁-D-L₂-[Aₘ]-X¹-X²-X³—[Bₙ] Formula XVI,

wherein [Aₘ]-X¹-X²-X³—[Bₙ] is the oligonucleotide of any one of formulas I-XV; C is a single-stranded oligonucleotide of 10-50 linked nucleosides in length; L₁ is a loop region; and D is a single-stranded oligonucleotide of 10-50 linked nucleosides in length; L₂ is an optional linker; wherein the oligonucleotide includes a duplex structure formed by C and D of between 10-50 linked nucleosides in length, wherein the duplex structure includes at least one mismatch between nucleotides of C and nucleotides of D, and wherein C or D includes at least one alternative nucleobase.

In some embodiments, C and D include at least one alternative nucleobase. In other embodiments, L₁ includes linked nucleosides. In yet another embodiment, L₁ consists of linked nucleosides. In some embodiments, L₁ includes at least one alternative nucleobase, at least one alternative internucleoside linkage, and/or at least one alternative sugar moiety. In some embodiments, C or D includes at least one alternative internucleoside linkage and/or at least one alternative sugar moiety. In some embodiments, C and D each independently includes at least one alternative internucleoside linkage and/or at least one alternative sugar moiety.

In some embodiments, the oligonucleotide includes the structure of Formula XVII:

C-L₁-D-L₂-[Aₘ]-X¹-X²-X³—[Bₙ] Formula XVII,

wherein [Aₘ]-X¹—X²-X³—[Bₙ] is the oligonucleotide of any one of Formulas I-XV; C is a single-stranded oligonucleotide of 10-50 linked nucleosides in length; L₁ is a loop region that does not consist of linked nucleosides; and D is a single-stranded oligonucleotide of 10-50 linked nucleosides in length; L₂ is an optional linker, wherein the oligonucleotide includes a duplex structure formed by C and D of between 10-50 linked nucleosides in length, and wherein the duplex structure includes at least one mismatch between nucleotides of C and nucleotides of D.

In some embodiments, L₁ has the structure of Formula XVIII:

F¹-(G¹)ⱼ-(H¹)ₖ-(G²)ₘ-(I)-(G³)ₙ-(H²)ₚ-(G⁴)_{q}-F² Formula XVIII,

wherein F¹ is a bond between the loop region and C; F² is a bond between D and [Aₘ] or between D and, optionally, the linker; G¹, G², G³, and G⁴ each, independently, is selected from optionally substituted C₁-C₂ alkyl, optionally substituted C1-C3 heteroalkyl, O, S, and NR^{N}; R^{N} is hydrogen, optionally substituted C₁₋₄ alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; j, k, m, n, p, and q are each, independently, 0 or 1; and I is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking F¹-(G¹)ⱼ-(H¹)k-(G²)ₘ-(I)-(G³)ₙ-(H²)ₚ-(G⁴)_{q}-F².

In some embodiments, L₁ includes a carbohydrate-containing linking moiety.

In some embodiments, C or D each includes at least one alternative nucleobase, at least one alternative internucleoside linkage, and/or at least one alternative sugar moiety. In some embodiments, C and D each includes at least one alternative nucleobase, at least one alternative internucleoside linkage, and/or at least one alternative sugar moiety.

In some embodiments, the oligonucleotide includes the structure of Formula XIX:

C-L₁-D-L₂-[Aₘ]-X¹-X²-X³—[Bₙ] Formula XIX,

wherein [Aₘ]-X¹-X²-X³—[Bₙ] is the oligonucleotide of any one of formulas I to XV; C is a single-stranded oligonucleotide of 10-50 linked nucleosides in length; L₁ is a loop region including at least one alternative nucleobase or at least one alternative internucleoside linkage; and D is a single-stranded oligonucleotide of 10-50 linked nucleosides in length; L₂ is an optional linker, wherein the oligonucleotide includes a duplex structure formed by C and D of between 10-50 linked nucleosides in length, and wherein the duplex structure includes at least one mismatch between nucleotides of C and nucleotides of D.

In some embodiments, L₁ includes at least one alternative nucleobase and at least one alternative internucleoside linkage.

In some embodiments, the oligonucleotide includes the structure of Formula XX:

C-L₁-D-L₂-[Aₘ]-X¹-X²-X³—[Bₙ] Formula XX,

wherein [Aₘ]-X¹-X²-X³—[Bₙ] is the oligonucleotide of any one of formulas I to XV; C is a single-stranded oligonucleotide of 10-50 linked nucleosides in length; L₁ is a loop region including at least one alternative sugar moiety, wherein the alternative sugar moiety is selected from the group consisting of a 2′-O—C₁-C₆ alkyl-sugar moiety, a 2′-amino-sugar moiety, a 2′-fluoro-sugar moiety, a 2′-O-MOE sugar moiety, an arabino nucleic acid (ANA) sugar moiety, a deoxyribose sugar moiety, and a bicyclic nucleic acid; D is a single-stranded oligonucleotide of 10-50 linked nucleosides in length; and L₂ is an optional linker, wherein the oligonucleotide includes a duplex structure formed by C and D of between 10-50 linked nucleosides in length, and wherein the duplex structure includes at least one mismatch between nucleotides of C and nucleotides of D.

In some embodiments, the bicyclic sugar moiety is selected from an oxy-LNA sugar moiety (also referred to as an “LNA sugar moiety”), a thio-LNA sugar moiety, an amino-LNA sugar moiety, a cEt sugar moiety, and an ethylene-bridged (ENA) sugar moiety. In some embodiments, the ANA sugar moiety is a 2′-fluoro-ANA sugar moiety.

In some embodiments, C or D includes at least one alternative nucleobase, at least one alternative internucleoside linkage, and/or at least one alternative sugar moiety. In some embodiments, C and D each includes at least one alternative nucleobase, at least one alternative internucleoside linkage, and/or at least one alternative sugar moiety. In some embodiments, C is complementary to at least 5 contiguous nucleobases of D. In some embodiments, at least 80% (e.g., at least 85%, at least 90%, at least 95%) of the nucleobases of C are complementary to the nucleobases of D.

In some embodiments, C includes a nucleobase sequence having at least 80% sequence identity to a nucleobase sequence set forth in any one of SEQ ID NO. 1, 4, 7, 10, 13, 16, 19, 22, 25, 28, 31, and 34.

In some embodiments, D includes a nucleobase sequence having at least 80% sequence identity to a nucleobase sequence set forth in any one of SEQ ID NOs. 2, 5, 8, 11, 14, 17, 20, 23, 26, 29, 32, and 35.

In some embodiments, C-L₁-D includes a nucleobase sequence having at least 80% sequence identity to a nucleobase sequence set forth in any one of SEQ ID NOs. 3, 6, 9, 12, 15, 18, 21, 24, 27, 30, 33, and 36.

In some embodiments, the at least one alternative nucleobase is selected from the group consisting of 5-methylcytosine, 5-hydroxycytosine, 5-methoxycytosine, N4-methylcytosine, N3-Methylcytosine, N4-ethylcytosine, pseudoisocytosine, 5-fluorocytosine, 5-bromocytosine, 5-iodocytosine, 5-aminocytosine, 5-ethynylcytosine, 5-propynylcytosine, pyrrolocytosine, 5-aminomethylcytosine, 5-hydroxymethylcytosine, naphthyridine, 5-methoxyuracil, pseudouracil, dihydrouracil, 2-thiouracil, 4-thiouracil, 2-thiothymine, 4-thiothymine, 5,6-dihydrothymine, 5-halouracil, 5-propynyluracil, 5-aminomethyluracil, 5-hydroxymethyluracil, hypoxanthine, 7-deazaguanine, 8-aza-7-deazaguanine, 7-aza-2,6-diaminopurine, thienoguanine, N1-methylguanine, N2-methylguanine, 6-thioguanine, 8-methoxyguanine, 8-allyloxyguanine, 7-aminomethyl-7-deazaguanine, 7-methylguanine, imidazopyridopyrimidine, 7-deazaadenine, 3-deazaadenine, 8-aza-7-deazaadenine, 8-aza-7-deazaadenine, N1-methyladenine, 2-methyladenine, N6-methyladenine, 7-methyladenine, 8-methyladenine, or 8-azidoadenine.

In some embodiments, the at least one alternative nucleobase is selected from the group consisting of 2-amino-purine, 2,6-diamino-purine, 3-deaza-adenine, 7-deaza-adenine, 7-methyl-adenine, 8-azido-adenine, 8-methyl-adenine, 5-hydroxymethyl-cytosine, 5-methyl-cytosine, pyrrolo-cytosine, 7-aminomethyl-7-deaza-guanine, 7-deaza-guanine, 7-methyl-guanine, 8-aza-7-deaza-guanine, thieno-guanine, hypoxanthine, 4-thio-uracil, 5-methoxy-uracil, dihydro-uracil, or pseudouracil.

In some embodiments, the at least one alternative internucleoside linkage is selected from the group consisting of a phosphorothioate internucleoside linkage, a 2′-alkoxy internucleoside linkage, and an alkyl phosphate internucleoside linkage. In some embodiments, the at least one alternative internucleoside linkage is at least one phosphorothioate internucleoside linkage.

In some embodiments, the at least one alternative sugar moiety is selected from the group consisting of a 2′-O-alkyl-sugar moiety, a 2′-O-methyl-sugar moiety, a 2′-amino-sugar moiety, a 2′-fluoro-sugar moiety, a 2′-O-MOE sugar moiety, an ANA sugar moiety deoxyribose sugar moiety, and a bicyclic nucleic acid. In some embodiments, the bicyclic sugar moiety is selected from an oxy-LNA sugar moiety, a thio-LNA sugar moiety, an amino-LNA sugar moiety, a cEt sugar moiety, and an ethylene-bridged (ENA) sugar moiety. In some embodiments, the ANA sugar moiety is a 2′-fluoro-ANA sugar moiety. In some embodiments, the at least one alternative sugar moiety is a 2′-O-methyl-sugar moiety, a 2′-fluoro-sugar moiety, or a 2′-O-MOE sugar moiety.

In some embodiments, the at least one mismatch is a paired A to C mismatch, a paired G to G mismatch, or a paired C to A mismatch. In some embodiments, the oligonucleotide includes at least two mismatches between nucleotides of C and nucleotides of D.

In some embodiments, the at least two mismatches are separated by at least three linked nucleosides. In some embodiments, the at least two mismatches are separated by three linked nucleosides.

In some embodiments, the at least one mismatch includes a nucleoside having an alternative nucleobase. In some embodiments, the alternative nucleobase has the structure:

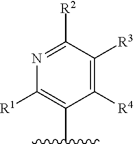

wherein R¹ is hydrogen, trifluoromethyl, optionally substituted amino, hydroxyl, or optionally substituted C₁-C₆ alkoxy; R² is hydrogen, optionally substituted amino, or optionally substituted C₁-C₆ alkyl; and R³ and R⁴ are, independently, hydrogen, halogen, or optionally substituted C₁-C₆alkyl, or a salt thereof.

In one embodiment, the oligonucleotides of the invention include those including an ADAR-recruiting domain having a structure of Formula XXXIV:

C-L₁-D, Formula XXXIV,

wherein C is a single-stranded oligonucleotide of about 10-50 linked nucleosides in length (e.g., about 10, 15, 20, 25, 30, 35, 40, 45, 46, 47, 48, 49, or 50 linked nucleosides in length), L₁ is a loop region, and D is a single-stranded oligonucleotide of about 10-50 linked nucleosides in length (e.g., about 10, 15, 20, 25, 30, 35, 40, 45, 46, 47, 48, 49, or 50 linked nucleosides in length).

In some embodiments, C includes a region that is complementary to D such that the two strands hybridize and form a duplex under suitable conditions. Generally, the duplex structure is between 5 and 50 linked nucleosides in length, e.g., between, 5-49, 5-45, 5-40, 5-35, 5-30, 5-25, 5-20, 5-15, 5-10, 5-6, 8-50, 8-45, 8-40, 8-35, 8-30, 8-25, 8-20, 8-15, 8-10, 15-50, 15-45, 15-40, 15-35, 15-30, 15-25, 15-20, 15-16, 20-50, 20-45, 20-40, 20-35, 20-30, 20-25, 25-50, 25-45, 25-40, 25-35, or 25-30 linked nucleosides in length. Ranges and lengths intermediate to the above-recited ranges and lengths are also contemplated to be part of the invention. In some embodiments, C is complementary to at least 5 contiguous nucleobases (e.g., 5, 10, 15, 20, 25, 30, or more contiguous nucleobases) of D, and the oligonucleotide forms a duplex structure of between 10-50 linked nucleosides in length (e.g., at least 10, 15, 20, 25, 30, 35, 40, 45, 46, 47, 48, 49, or 50 linked nucleosides in length).

In some embodiments, the duplex structure includes at least one mismatch between nucleotides of C and nucleotides of D (e.g., at least 1, 2, 3, 4, or 5 mismatches). In some embodiments, the mismatch is a paired A to C mismatch. In some embodiments, the A nucleoside of the A to C mismatch is on the C strand and the C nucleoside of the A to C mismatch is on the D strand. In some embodiments, the A nucleoside of the A to C mismatch is on the D strand and the C nucleoside of the A to C mismatch is on the C strand. In other embodiments, the mismatch is a paired G-to-G mismatch. In still yet other embodiments, the mismatch is a paired C to A mismatch. In some embodiments, the C nucleoside of the C to A mismatch is on the C strand and the A nucleoside of the C to A mismatch is on the D strand. In some embodiments, the C nucleoside of the C to A mismatch is on the D strand and the A nucleoside of the C to A mismatch is on the C strand. In some embodiments, the mismatch is a paired I to I mismatch. In some embodiments, the mismatch is a paired I to G mismatch. In some embodiments, the I nucleoside of the I to G mismatch is on the C strand and the G nucleoside of the I to G mismatch is on the D strand. In some embodiments, the I nucleoside of the I to G mismatch is on the D strand and the G nucleoside of the I to G mismatch is on the C strand. In some embodiments, the mismatch is a paired G to I mismatch. In some embodiments, the G nucleoside of the G to I mismatch is on the C strand and the I nucleoside of the G to I mismatch is on the D strand. In some embodiments, the G nucleoside of the G to I mismatch is on the D strand and the I nucleoside of the G to I mismatch is on the C strand. In some embodiments, the mismatch includes a nucleoside having an alternative nucleobase. In some embodiments, the alternative nucleobase has the structure:

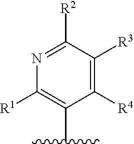

wherein R¹ is hydrogen, trifluoromethyl, optionally substituted amino, hydroxyl, or optionally substituted C₁-C₆ alkoxy; R² is hydrogen, optionally substituted amino, or optionally substituted C₁-C₆ alkyl; and R³ and R⁴ are, independently, hydrogen, halogen, or optionally substituted C₁-C₆ alkyl, or a salt thereof. In some embodiments, R¹ is a hydrogen bond donor group (e.g., a hydroxyl group, an amino group). In some embodiments, R¹ is a hydrogen bond accepting group (e.g., an alkoxy group).

In some embodiments, the duplex structure includes two mismatches. In some embodiments, the mismatches are at least three linked nucleosides apart. For example, when mismatches are “separated by 3 nucleotides,” the oligonucleotide includes the structure M₁-N₁N₂-N₃-M₂, where M₁ is the first mismatch, N₁, N₂, and N₃ are paired nucleobases, and M₂ is the second mismatch. In some embodiments M₁ is a paired A to C mismatch and M₂ is a paired G-to-G mismatch.

In some embodiments, the loop region, Li, includes linked nucleosides. In some embodiments, L₁ includes at least one alternative nucleobase, at least one alternative internucleoside linkage, and/or at least one alternative sugar moiety.

In other embodiments, the loop region has the structure of Formula XVIII:

F¹-(G¹)ⱼ-(H¹)ₖ-(G²)ₘ-(I)-(G³)ₙ-(H²)ₚ-(G⁴)_{q}-F² Formula XVIII,

wherein F¹ is a bond between the loop region and C; F² is a bond between D and a nucleotide or between D and, optionally, a linker; G¹, G², G³, and G⁴ each, independently, is selected from optionally substituted C₁-C₂ alkyl, optionally substituted C₁-C₃ heteroalkyl, O, S, and NR^{N}; R^{N} is hydrogen, optionally substituted C₁-4 alkyl, optionally substituted C₂₋₄ alkenyl, optionally substituted C₂₋₄ alkynyl, optionally substituted C₂₋₆ heterocyclyl, optionally substituted C₆₋₁₂ aryl, or optionally substituted C₁₋₇ heteroalkyl; C¹ and C² are each, independently, selected from carbonyl, thiocarbonyl, sulphonyl, or phosphoryl; j, k, m, n, p, and q are each, independently, 0 or 1; and I is optionally substituted C₁₋₁₀ alkyl, optionally substituted C₂₋₁₀ alkenyl, optionally substituted C₂₋₁₀ alkynyl, optionally substituted C₂₋₆ heterocyclyl, optionally substituted C₆₋₁₂ aryl, optionally substituted C₂-C₁₀ polyethylene glycol, or optionally substituted C₁₋₁₀ heteroalkyl, or a chemical bond linking F¹-(G¹)ⱼ-(H¹)ₖ-(G²)ₘ-(I)-(G³)ₙ-(H²)ₚ-(G⁴)_{q}-F². In some embodiments, the linker is optional.

In some embodiments, the loop region, L₁ includes a carbohydrate-containing linking moiety.

In one embodiment, one or more of the nucleotides of the oligonucleotides of the invention, is naturally-occurring, and does not include, e.g., chemical modifications and/or conjugations known in the art and described herein. In another embodiment, one or more of the nucleotides of an oligonucleotide of the invention is chemically modified to enhance stability or other beneficial characteristics (e.g., alternative nucleotides). Without being bound by theory, it is believed that certain modification can increase nuclease resistance and/or serum stability, or decrease immunogenicity. For example, polynucleotides of the invention may contain nucleotides found to occur naturally in DNA or RNA (e.g., adenine, thymidine, guanosine, cytidine, uridine, or inosine) or may contain nucleotides which have one or more chemical modifications to one or more components of the nucleotide (e.g., the nucleobase, sugar, or phospho-linker moiety). Oligonucleotides of the invention may be linked to one another through naturally-occurring phosphodiester bonds, or may be modified to be covalently linked through phosphorothiorate, 3′-methylenephosphonate, 5′-methylenephosphonate, 3′-phosphoamidate, 2′-5′ phosphodiester, guanidinium, S-methylthiourea, or peptide bonds.

In some embodiments, C includes at least one alternative nucleobase, at least one alternative internucleoside linkage, and/or at least one alternative sugar moiety. In other embodiments, D includes at least one alternative nucleobase, at least one alternative internucleoside linkage, and/or at least one alternative sugar moiety. In some embodiments, both C and D each include at least one alternative nucleobase, at least one alternative internucleoside linkage, and/or at least one alternative sugar moiety.

In certain embodiments of the invention, substantially all of the nucleotides of an oligonucleotide of the invention are alternative nucleotides. In other embodiments of the invention, all of the nucleotides of an oligonucleotide of the invention are alternative nucleotides. Oligonucleotides of the invention in which “substantially all of the nucleotides are alternative nucleotides” are largely but not wholly modified and can include no more than 5, 4, 3, 2, or 1 naturally-occurring nucleotides. In still other embodiments of the invention, an oligonucleotide of the invention can include no more than 5, 4, 3, 2, or 1 alternative nucleotides.

In one embodiment, the oligonucleotides of the invention include an ADAR-recruiting domain having the structure of Formula XXXIV, wherein C is a single-stranded oligonucleotide of 10-50 linked nucleosides in length, L₁ is a loop region, and D is a single-stranded oligonucleotide of 10-50 linked nucleosides in length. In some embodiments, C is complementary to at least 5 contiguous nucleobases of D, and the oligonucleotide includes a duplex structure formed by C and D of between 10-50 linked nucleosides in length. In some embodiments, the duplex structure includes at least one mismatch. In some embodiments, C or D includes at least one alternative nucleobase. In some embodiments, C and D each include at least one alternative nucleobase. In some embodiments, C and/or D, independently, further include at least one alternative internucleoside linkage and/or at least one alternative sugar moiety. In some embodiments, L₁ includes linked nucleotides. In other embodiments, Li consists of linked nucleosides. In some embodiments, L₁ includes at least one alternative nucleobase, at least one alternative internucleoside linkage, and/or at least one alternative sugar moiety.

In another embodiment, the oligonucleotides of the invention include an ADAR-recruiting domain having the structure of Formula XXXIV, wherein C is a single-stranded oligonucleotide of 10-50 linked nucleosides in length, L₁ is a loop region that does not consist of linked nucleosides, and D is a single-stranded oligonucleotide of 10-50 linked nucleosides in length. In some embodiments, C is complementary to at least 5 contiguous nucleobases of D, and the oligonucleotide includes a duplex structure formed by C and D of between 10-50 linked nucleosides in length. In some embodiments, the duplex structure includes at least one mismatch. In some embodiments, L₁ has the structure of Formula VIII, as described herein. In some embodiments, L₁ includes a carbohydrate-containing linking moiety. In some embodiments, C and/or D, independently, include at least one alternative nucleobase, at least one alternative internucleoside linkage, and/or at least one alternative sugar moiety.

In another embodiment, the oligonucleotides of the invention include an ADAR-recruiting domain having the structure of Formula XXXIV, wherein C is a single-stranded oligonucleotide of 10-50 linked nucleosides in length, L₁ is a loop region including at least one alternative nucleobase or at least one alternative internucleoside linkage, and D is a single-stranded oligonucleotide of 10-50 linked nucleosides in length. In some embodiments, C is complementary to at least 5 contiguous nucleobases of D, and the oligonucleotide includes a duplex structure formed by C and D of between 10-50 linked nucleosides in length. In some embodiments, the duplex structure includes at least one mismatch. In some embodiments, Li includes at least one alternative nucleobase and at least one alternative internucleoside linkage.

In another embodiment, the oligonucleotides of the invention include an ADAR-recruiting domain having the structure of Formula XXXIV, wherein C is a single-stranded oligonucleotide of 10-50 linked nucleosides in length, L₁ is a loop region including, at least one alternative sugar moiety that is not a 2′-O-methyl sugar moiety (e.g., the alternative sugar moiety is selected from the group consisting of a 2′-O—C₁-C₆ alkyl-sugar moiety, a 2′-amino-sugar moiety, a 2′-fluoro-sugar moiety, a 2′-O-MOE sugar moiety, an LNA sugar moiety, an arabino nucleic acid (ANA) sugar moiety, a 2′-fluoro-ANA sugar moiety, a deoxyribose sugar moiety, and a bicyclic nucleic acid), and D is a single-stranded oligonucleotide of 10-50 linked nucleosides in length. In some embodiments, C is complementary to at least 5 contiguous nucleobases of D, and the oligonucleotide includes a duplex structure formed by C and D of between 10-50 linked nucleosides in length. In some embodiments, the duplex structure includes at least one mismatch. In some embodiments, C and/or D, independently, include at least one alternative nucleobase, at least one alternative internucleoside linkage, and/or at least one alternative sugar moiety.

In some embodiments, C includes a nucleobase sequence having at least 50% sequence identity (e.g., at least 50%, at least 60%, at least 70%, at least 80%, at least 85%, at least 90%, at least 95%, at least 96%, at least 97%, at least 98%, at least 99%, or 100% sequence identity) to a nucleobase sequence set forth in of any one of SEQ ID NOs. 1, 4, 7, 10, 13, 16, 19, 22, 25, 28, 31, and 34, and D includes a nucleobase sequence complementary to the nucleobase sequence of C, wherein the sequence includes at least one mismatch as described herein. In other embodiments, D includes a nucleobase sequence having at least 50% sequence identity (e.g., at least 50%, at least 60%, at least 70%, at least 80%, at least 85%, at least 90%, at least 95%, at least 96%, at least 97%, at least 98%, at least 99%, or 100% sequence identity) to a nucleobase sequence set forth in of any one of SEQ ID NOs. 2, 5, 8, 11, 14, 17, 20, 23, 26, 29, 32, and 35, and C includes a nucleobase sequence complementary to the nucleobase sequence of C, wherein the sequence includes at least one mismatch as described herein. In some embodiments, C-L₁-D includes a nucleobase sequence having at least 50% sequence identity (e.g., at least 50%, at least 60%, at least 70%, at least 80%, at least 85%, at least 90%, at least 95%, at least 96%, at least 97%, at least 98%, at least 99%, or 100% sequence identity) to a nucleobase sequence set forth in of any one of SEQ ID NOs. 3, 6, 9, 12, 15, 18, 21, 24, 27, 30, 33, and 36, wherein the sequence includes at least one mismatch as described herein.

Nucleobase sequences of SEQ ID NOs. 1-36 are provided below:

TABLE 3

| | |
|---|---|
| GGUGAAUAGUAUAACAAUAU | SEQ ID NO. 1 |
| AUGUUGUUAUAGUAUCCACC | SEQ ID NO. 2 |
| GGUGAAUAGUAUAACAAUAUGCUAAAUGUUGUUAU | SEQ ID NO. 3 |
| AGUAUCCACC | |
| GGUGAAGAGGAGAACAAUAU | SEQ ID NO. 4 |
| AUGUUGUUCUCGUCUCCACC | SEQ ID NO. 5 |
| GGUGAAGAGGAGAACAAUAUGCUAAAUGUUGUUCU | SEQ ID NO. 6 |
| CGUCUCCACC | |
| GGUGUCGAGAAGAGGAGAACAAUAU | SEQ ID NO. 7 |
| AUGUUGUUCUCGUCUCCUCGACACC | SEQ ID NO. 8 |
| GGUGUCGAGAAGAGGAGAACAAUAUGCUAAAUGUU | SEQ ID NO. 9 |
| GUUCUCGUCUCCUCGACACC | |
| GGGUGGAAUAGUAUAACAAUAU | SEQ ID NO. 10 |
| AUGUUGUUAUAGUAUCCCACCU | SEQ ID NO. 11 |
| GGGUGGAAUAGUAUAACAAUAUGCUAAAUGUUGUU | SEQ ID NO. 12 |
| AUAGUAUCCCACCU | |
| GUGGAAUAGUAUAACAAUAU | SEQ ID NO. 13 |
| AUGUUGUUAUAGUAUCCCAC | SEQ ID NO. 14 |
| GUGGAAUAGUAUAACAAUAUGCUAAAUGUUGUUAU | SEQ ID NO. 15 |
| AGUAUCCCAC | |
| GGUGUCGAGAAUAGUAUAACAAUAU | SEQ ID NO. 16 |
| AUGUUGUUAUAGUAUCCUCGACACC | SEQ ID NO. 17 |
| GGUGUCGAGAAUAGUAUAACAAUAUGCUAAAUGUU | SEQ ID NO. 18 |
| GUUAUAGUAUCCUCGACACC | |
| GGGUGGAAUAGUAUAACAAUAU | SEQ ID NO. 19 |
| AUGUUGUUAUAGUAUCCCACCU | SEQ ID NO. 20 |
| GGGUGGAAUAGUAUAACAAUAUGCUAAAUGUUGUU | SEQ ID NO. 21 |
| AUAGUAUCCCACCU | |
| GGGUGGAAUAGUAUACCA | SEQ ID NO. 22 |
| UGGUAUAGUAUCCCACCU | SEQ ID NO. 23 |
| GGGUGGAAUAGUAUACCAUUCGUGGUAUAGUAUCC | SEQ ID NO. 24 |
| CACCU | |
| GUGGGUGGAAUAGUAUACCA | SEQ ID NO. 25 |
| UGGUAUAGUAUCCCACCUAC | SEQ ID NO. 26 |
| GUGGGUGGAAUAGUAUACCAUUCGUGGUAUAGUAU | SEQ ID NO. 27 |
| CCCACCUAC | |
| UGGGUGGAAUAGUAUACCA | SEQ ID NO. 28 |
| UGGUAUAGUAUCCCACCUA | SEQ ID NO. 29 |
| UGGGUGGAAUAGUAUACCAUUCGUGGUAUAGUAUC | SEQ ID NO. 30 |
| CCACCUA | |
| GGUGGAAUAGUAUACCA | SEQ ID NO. 31 |
| UGGUAUAGUAUCCCACC | SEQ ID NO. 32 |
| GGUGGAAUAGUAUACCAUUCGUGGUAUAGUAUCCC | SEQ ID NO. 33 |
| ACC | |
| GUGGAAUAGUAUACCA | SEQ ID NO. 34 |
| UGGUAUAGUAUCCCAC | SEQ ID NO. 35 |
| GUGGAAUAGUAUACCAUUCGUGGUAUAGUAUCCCA | SEQ ID NO. 36 |
| C | |

It will be understood that, although the sequences in SEQ ID NOs. 1-36 are described as unmodified and/or un-conjugated sequences, the RNA of the oligonucleotides of the invention may include any one of the sequences set forth in SEQ ID NOs. 1-36 that is an alternative nucleoside and/or conjugated as described in detail below.

In some embodiments, the oligonucleotide of the invention may further include a 5′ cap structure. In some embodiments, the 5′ cap structure is a 2,2,7-trimethylguanosine cap.

An oligonucleotide of the invention can be synthesized by standard methods known in the art as further discussed below, e.g., by use of an automated DNA synthesizer, such as are commercially available from, for example, Biosearch, Applied Biosystems, Inc.

The oligonucleotide compound can be prepared using solution-phase or solid-phase organic synthesis or both. Organic synthesis offers the advantage that the oligonucleotide including unnatural or alternative nucleotides can be easily prepared. Single-stranded oligonucleotides of the invention can be prepared using solution-phase or solid-phase organic synthesis or both.

Further, it is contemplated that for any sequence identified herein, further optimization could be achieved by systematically either adding or removing linked nucleosides to generate longer or shorter sequences. Further still, such optimized sequences can be adjusted by, e.g., the introduction of alternative nucleosides, alternative sugar moieties, and/or alternative internucleosidic linkages as described herein or as known in the art, including alternative nucleosides, alternative sugar moieties, and/or alternative internucleosidic linkages as known in the art and/or discussed herein to further optimize the molecule (e.g., increasing serum stability or circulating half-life, increasing thermal stability, enhancing transmembrane delivery, targeting to a particular location or cell type, and/or increasing interaction with RNA editing enzymes (e.g., ADAR)).

In some embodiments, the one or more ADAR-recruiting domains are GluR2 ADAR-recruiting domains. In some embodiments, the GluR2 ADAR-recruiting domain has the nucleotide sequence of SEQ ID NO. 37, as shown below in the 5′ to 3′ direction:

(SEQ ID NO. 37)
GGUGAAUAGUAUAACAAUAUGCUAAAUGUUGUUAUAGUAUCCACC

In some embodiments, the oligonucleotide includes the structure of Formula XXI (SEQ ID NO: 37), as shown below:

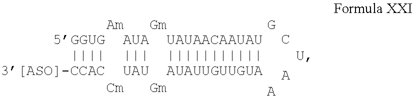

wherein [ASO] includes any of the oligonucleotides of the instant invention, wherein m designates a mismatched nucleotide. In some embodiments, the GluR2 ADAR-recruiting domain has the nucleotide sequence of SEQ ID NO. 38, as shown below in the 5′ to 3′ direction:

(SEQ ID NO. 38)
GGUGAAGAGGAGAACAAUAUGCUAAAUGUUGUUCUCGUCUCCACC

In some embodiments, the oligonucleotide includes the structure of Formula XXII (SEQ ID NO: 38), as shown below:

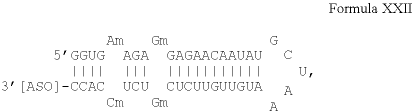

wherein [ASO] includes any of the oligonucleotides of the instant invention, wherein m designates a mismatched nucleotide. In some embodiments, the GluR2 ADAR-recruiting domain has the nucleotide sequence of SEQ ID NO. 39, as shown below in the 5′ to 3′ direction:

(SEQ ID NO. 39)
GGUGUCGAGAAGAGGAGAACAAUAUGCUAAAUGUUGUUCUCGUCUCCUC
GACACC

In some embodiments, the oligonucleotide includes the structure of Formula XXIII (SEQ ID NO: 39), as shown below:

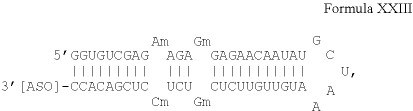

wherein [ASO] includes any of the oligonucleotides of the instant invention, wherein m designates a mismatched nucleotide.

In some embodiments, the GluR2 ADAR-recruiting domain has the nucleotide sequence of SEQ ID NO. 40, as shown below in the 5′ to 3′ direction:

(SEQ ID NO. 40)
*s*s*G**GAGAAGAGGAGAA*AA*A*G**AAA*G**G*****G*****
**GA*A**

wherein * is a 2′-O-methyl nucleotide and s is a phosphorothioate internucleoside linkage between two linked nucleotides. In some embodiments, the oligonucleotide includes the structure of Formula XXIV (SEQ ID NO: 40), as shown below:

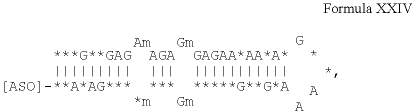

wherein [ASO] includes any one of the oligonucleotides presented herein, wherein * is a 2′-O-methyl nucleotide, wherein s is a phosphorothioate internucleoside linkage, wherein m designates a mismatched nucleotide. In some embodiments, the ADAR-recruiting domains further include at least one nuclease-resistant nucleotide (e.g., 2′-O-methyl nucleotide). In some embodiments, the ADAR-recruiting domains include at least one alternative internucleoside linkage (e.g., a phosphorothioate internucleoside linkage). In some embodiments, the GluR2 ADAR-recruiting domain has the nucleotide sequence of SEQ ID NO. 41, as shown below in the 5′ to 3′ direction:

(SEQ ID NO. 41)
GGGUGGAAUAGUAUAACAAUAUGCUAAAUGUUGUUAUAGUAUCCCACCU

In some embodiments, the oligonucleotide includes the structure of Formula XXV (SEQ ID NO: 41), as shown below:

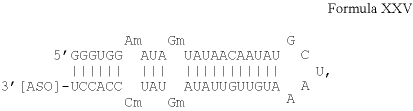

wherein [ASO] includes any of the oligonucleotides of the instant invention, wherein m designates a mismatched nucleotide. In some embodiments, the GluR2 ADAR-recruiting domain has the nucleotide sequence of SEQ ID NO. 42, as shown below in the 5′ to 3′ direction:

(SEQ ID NO. 42)
GUGGAAUAGUAUAACAAUAUGCUAAAUGUUGUUAUAGUAUCCCAC

In some embodiment, the oligonucleotide includes the structure formula XXVI(SEQ ID NO: 42), as shown below:

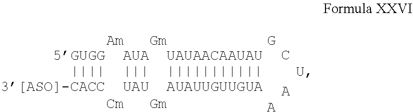

wherein [ASO] includes any of the oligonucleotides of the instant invention, wherein m designates a mismatched nucleotide. In some embodiments, the GluR2 ADAR-recruiting domain has the nucleotide sequence of SEQ ID NO. 43, as shown below in the 5′ to 3′ direction:

(SEQ ID NO. 43)
GGUGUCGAGAAUAGUAUAACAAUAUGCUAAAUGUUGUUAUAGUAUCCUC
GACACC

In some embodiments, the oligonucleotide includes the structure of Formula XXVII (SEQ ID NO: 43), as shown below:

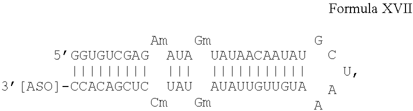

wherein [ASO] includes any of the oligonucleotides of the instant invention, wherein m designates a mismatched nucleotide. In some embodiments, the GluR2 ADAR-recruiting domain has the nucleotide sequence of SEQ ID NO. 44, as shown below in the 5′ to 3′ direction:

(SEQ ID NO. 44)
GGGUGGAAUAGUAUAACAAUAUGCUAAAUGUUGUUAUAGUAUCCCACCU

In some embodiments, the oligonucleotide includes the structure of Formula XXVIII (SEQ ID NO: 44), as shown below:

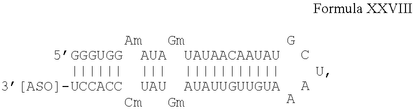

wherein [ASO] includes any of the oligonucleotides of the instant invention, wherein m designates a mismatched nucleotide. In some embodiments, the GluR2 ADAR-recruiting domain has the nucleotide sequence of SEQ ID NO. 45, as shown below in the 5′ to 3′ direction:

(SEQ ID NO. 45)
GGGUGGAAUAGUAUACCAUUCGUGGUAUAGUAUCCCACCU

In some embodiments, the oligonucleotide includes the structure of Formula XXIX (SEQ ID NO: 45), as shown below:

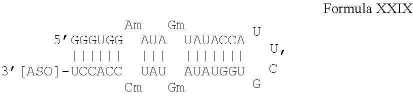

wherein [ASO] includes any of the oligonucleotides of the instant invention, wherein m designates a mismatched nucleotide. In some embodiments, the GluR2 ADAR-recruiting domain has the nucleotide sequence of SEQ ID NO. 46, as shown below in the 5′ to 3′ direction:

(SEQ ID NO. 46)
GUGGGUGGAAUAGUAUACCAUUCGUGGUAUAGUAUCCCACCUAC

In some embodiments, the oligonucleotide includes the structure of Formula XXX (SEQ ID NO: 46), as shown below:

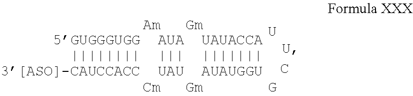

wherein [ASO] includes any of the oligonucleotides of the instant invention, wherein m designates a mismatched nucleotide. In some embodiments, the GluR2 ADAR-recruiting domain has the nucleotide sequence of SEQ ID NO. 47, as shown below in the 5′ to 3′ direction:

(SEQ ID NO. 47)
UGGGUGGAAUAGUAUACCAUUCGUGGUAUAGUAUCCCACCUA

In some embodiments, the oligonucleotide includes the structure of Formula XXXI (SEQ ID NO: 47), as shown below:

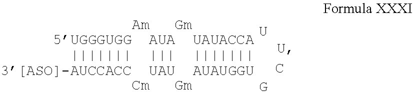

wherein [ASO] includes any of the oligonucleotides of the instant invention, wherein m designates a mismatched nucleotide. In some embodiments, the GluR2 ADAR-recruiting domain has the nucleotide sequence of SEQ ID NO. 48, as shown below in the 5′ to 3′ direction:

(SEQ ID NO. 48)
GGUGGAAUAGUAUACCAUUCGUGGUAUAGUAUCCCACC

In some embodiments, the oligonucleotide includes the structure of Formula XXXII (SEQ ID NO: 48), as shown below:

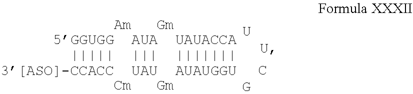

wherein [ASO] includes any of the oligonucleotides of the instant invention, wherein m designates a mismatched nucleotide. In some embodiments, the GluR2 ADAR-recruiting domain has the nucleotide sequence of SE ID NO. 49, as shown below in the 5′ to 3′ direction:

(SEQ ID NO. 49)
GUGGAAUAGUAUACCAUUCGUGGUAUAGUAUCCCAC

In some embodiments, the oligonucleotide includes the structure of Formula XXXIII (SEQ ID NO: 49), as shown below:

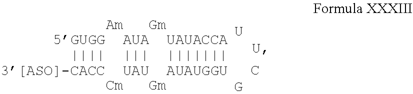

wherein [ASO] includes any of the oligonucleotides of the instant invention, wherein m designates a mismatched nucleotide.

In some embodiments, the ADAR-recruiting domains are Z-DNA ADAR-recruiting domains. In some embodiments, the ADAR-recruiting domains are MS2 ADAR-recruiting domains. In some embodiments, an MS2 bacteriophage stem-loop structure may be used as an ADAR-recruiting domain (e.g., and MS2 ADAR-recruiting domain). MS2 stem-loops are known to bind the MS2 bacteriophage coat protein, which when fused to the deaminase domain of ADAR (e.g. an ADAR fusion protein) can be used for target-specific deamination. In some embodiments, the MS2 ADAR-recruiting domain has the nucleotide sequence of SEQ ID NO. 50, as shown below in the 5′ to 3′ direction:

(SEQ ID NO. 50)
ACATGAGGATCACCCATGT

In some embodiments, an ADAR fusion protein is administered to the cell or to the subject using an expression vector construct including a polynucleotide encoding an ADAR fusion protein. In some embodiments, the ADAR fusion protein includes a deaminase domain of ADAR fused to an MS2 bacteriophage coat protein. In some embodiments, the deaminase domain of ADAR is a deaminase domain of ADAR1. In some embodiments, the deaminase domain of ADAR is a deaminase domain of ADAR2. The ADAR fusion protein may be a fusion protein described in Katrekar et al. Nature Methods, 16(3): 239-42 (2019), the ADAR fusion protein of which is herein incorporated by reference

The nucleic acids featured in the invention can be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Alternative nucleotides and nucleosides include those with modifications including, for example, end modifications, e.g., 5′-end modifications (phosphorylation, conjugation, inverted linkages) or 3′-end modifications (conjugation, DNA nucleotides, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; and/or backbone modifications, including modification or replacement of the phosphodiester linkages. The nucleobase may also be an isonucleoside in which the nucleobase is moved from the C1 position of the sugar moiety to a different position (e.g. C2, C3, C4, or C5). Specific examples of oligonucleotide compounds useful in the embodiments described herein include, but are not limited to alternative nucleosides containing modified backbones or no natural internucleoside linkages. Nucleotides and nucleosides having modified backbones include, among others, those that do not have a phosphorus atom in the backbone. For the purposes of this specification, and as sometimes referenced in the art, alternative RNAs that do not have a phosphorus atom in their internucleoside backbone can also be considered to be oligonucleosides. In some embodiments, an oligonucleotide will have a phosphorus atom in its internucleoside backbone.

Alternative internucleoside linkages include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boronophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts, and free acid forms are also included.

Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. No. RE39464, the entire contents of each of which are hereby incorporated herein by reference.

Alternative internucleoside linkages that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S, and CH₂ component parts.

Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, the entire contents of each of which are hereby incorporated herein by reference.

In other embodiments, suitable oligonucleotides include those in which both the sugar and the internucleoside linkage, i.e., the backbone, of the nucleotide units are replaced. The base units are maintained for hybridization with an appropriate nucleic acid target compound. One such oligomeric compound, a mimetic that has been shown to have excellent hybridization properties, is referred to as a peptide nucleic acid (PNA). In PNA compounds, the sugar of a nucleoside is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, the entire contents of each of which are hereby incorporated herein by reference. Additional PNA compounds suitable for use in the oligonucleotides of the invention are described in, for example, in Nielsen et al., Science, 1991, 254, 1497-1500.

Some embodiments featured in the invention include oligonucleotides with phosphorothioate backbones and oligonucleotides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂-[wherein the native phosphodiester backbone is represented as —O—P—O—CH₂-] of the above-referenced U.S. Pat. No. 5,489,677, and the amide backbones of the above-referenced U.S. Pat. No. 5,602,240. In some embodiments, the oligonucleotides featured herein have morpholino backbone structures of the above-referenced U.S. Pat. No. 5,034,506. In other embodiments, the oligonucleotides described herein include phosphorodiamidate morpholino oligomers (PMO), in which the deoxyribose moiety is replaced by a morpholine ring, and the charged phosphodiester inter-subunit linkage is replaced by an uncharged phophorodiamidate linkage, as described in Summerton, et al., Antisense Nucleic Acid Drug Dev. 1997, 7:63-70.

Alternative nucleosides and nucleotides can also contain one or more substituted sugar moieties. The oligonucleotides, e.g., oligonucleotides, featured herein can include one of the following at the 2′-position: OH; F; O-, S-, or N-alkyl; O-, S-, or N-alkenyl; O-, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary suitable modifications include —O[(CH₂)ₙO]ₘCH₃, —O(CH₂)ₙOCH₃, —O(CH₂)ₙ—NH₂, —O(CH₂)ₙCH₃, —O(CH₂)ₙ—ONH₂, and —O(CH₂)ₙ—ON[(CH₂)·CH₃]₂, where n and m are from 1 to about 10. In other embodiments, oligonucleotides include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some embodiments, the modification includes a 2′-methoxyethoxy (2′-O—CH₂CH₂OCH₃, also known as 2′-O-(2-methoxyethyl) or 2′-O-MOE) (Martin et al., Helv. Chin. Acta, 1995, 78:486-504) i.e., an alkoxy-alkoxy group. 2′-O-MOE nucleosides confer several beneficial properties to oligonucleotides including, but not limited to, increased nuclease resistance, improved pharmacokinetics properties, reduced non-specific protein binding, reduced toxicity, reduced immunostimulatory properties, and enhanced target affinity as compared to unmodified oligonucleotides.

Another exemplary alternative contains 2′-dimethylaminooxyethoxy, i.e., a —O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—(CH₂)₂—O—(CH₂)₂—N(CH₃)₂. Further exemplary alternatives include: 5′-Me-2′-F nucleotides, 5′-Me-2′-OMe nucleotides, 5′-Me-2′-deoxynucleotides, (both R and S isomers in these three families); 2′-alkoxyalkyl; and 2′-NMA (N-methylacetamide).

Other alternatives include 2′-methoxy (2′-OCH₃), 2′-aminopropoxy (2′-OCH₂CH₂CH₂NH₂) and 2′-fluoro (2′-F). Similar modifications can also be made at other positions on the nucleosides and nucleotides of an oligonucleotide, particularly the 3′ position of the sugar on the 3′ terminal nucleotide or in 2′-5′ linked oligonucleotides and the 5′ position of 5′ terminal nucleotide. Oligonucleotides can also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920. The entire contents of each of the foregoing are hereby incorporated herein by reference.

An oligonucleotide for use in the methods of the present invention can also include nucleobase (often referred to in the art simply as “base”) alternatives (e.g., modifications or substitutions). Unmodified or natural nucleobases include the purine bases adenine (A) and guanine (G), and the pyrimidine bases thymine (T), cytosine (C) and uracil (U). Alternative nucleobases include other synthetic and natural nucleobases such as 5-methylcytosine, 5-hydroxymethylcytosine, 5-formylcytosine, 5-carboxycytosine, pyrrolocytosine, dideoxycytosine, uracil, 5-methoxyuracil, 5-hydroxydeoxyuracil, dihydrouracil, 4-thiouracil, pseudouracil, 1-methyl-pseudouracil, deoxyuracil, 5-hydroxybutynl-2′-deoxyuracil, xanthine, hypoxanthine, 7-deaza-xanthine, thienoguanine, 8-aza-7-deazaguanine, 7-methylguanine, 7-deazaguanine, 6-aminomethyl-7-deazaguanine, 8-aminoguanine, 2,2,7-trimethylguanine, 8-methyladenine, 8-azidoadenine, 7-methyladenine, 7-deazaadenine, 3-deazaadenine, 2,6-diaminopurine, 2-aminopurine, 7-deaza-8-aza-adenine, 8-amino-adenine, thymine, dideoxythymine, 5-nitroindole, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 8-azaguanine and 8-azaadenine, and 3-deazaguanine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., (1991) Angewandte Chemie, International Edition, 30:613, and those disclosed by Sanghvi, Y S., Chapter 15, Antisense Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines, and N-2, N-6 and 0-6 substituted purines, including 2-aminopropyladenine, 5-propynyluracil, and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., Antisense Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted alternative nucleobases as well as other alternative nucleobases include, but are not limited to, the above noted U.S. Pat. Nos. 3,687,808, 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 5,750,692; 6,015,886; 6,147,200; 6,166,197; 6,222,025; 6,235,887; 6,380,368; 6,528,640; 6,639,062; 6,617,438; 7,045,610; 7,427,672; and 7,495,088, the entire contents of each of which are hereby incorporated herein by reference.

In other embodiments, the sugar moiety in the nucleotide may be a ribose molecule, optionally having a 2′-O-methyl, 2′-O-MOE, 2′-F, 2′-amino, 2′-O-propyl, 2′-aminopropyl, or 2′-OH modification.

An oligonucleotide for use in the methods of the present invention can include one or more bicyclic sugar moieties. A “bicyclic sugar” is a furanosyl ring modified by the bridging of two atoms. A “bicyclic nucleoside” (“BNA”) is a nucleoside having a sugar moiety including a bridge connecting two carbon atoms of the sugar ring, thereby forming a bicyclic ring system. In certain embodiments, the bridge connects the 4′-carbon and the 2′-carbon of the sugar ring. Thus, in some embodiments an agent of the invention may include one or more locked nucleosides. A locked nucleoside is a nucleoside having a modified ribose moiety in which the ribose moiety includes an extra bridge connecting the 2′ and 4′ carbons. In other words, a locked nucleoside is a nucleoside including a bicyclic sugar moiety including a 4′-CH₂—O-2′ bridge. This structure effectively “locks” the ribose in the 3′-endo structural conformation. The addition of locked nucleosides to oligonucleotides has been shown to increase oligonucleotide stability in serum, and to reduce off-target effects (Grunweller, A. et al., (2003) Nucleic Acids Research 31(12):3185-3193). Examples of bicyclic nucleosides for use in the polynucleotides of the invention include without limitation nucleosides including a bridge between the 4′ and the 2′ ribosyl ring atoms. In certain embodiments, the polynucleotide agents of the invention include one or more bicyclic nucleosides including a 4′ to 2′ bridge. Examples of such 4′ to 2′ bridged bicyclic nucleosides, include but are not limited to 4′-(CH₂)—O-2′ (LNA); 4′-(CH₂)—S-2′; 4′-(CH₂)₂—O-2′ (ENA); 4′-CH(CH₃)—O-2′ (also referred to as “constrained ethyl” or “cEt”) and 4′-CH(CH₂OCH₃)—O-2′ (and analogs thereof; see, e.g., U.S. Pat. No. 7,399,845); 4′-C(CH₃)(CH₃)—O-2′ (and analogs thereof; see e.g., U.S. Pat. No. 8,278,283); 4′-CH₂—N(OCH₃)-2′ (and analogs thereof; see e.g., U.S. Pat. No. 8,278,425); 4′-CH₂—O—N(CH₃)₂-2′ (see, e.g., U.S. Patent Publication No. 2004/0171570); 4′-CH₂—N(R)—O-2′, wherein R is H, C₁-C₁₂ alkyl, or a protecting group (see, e.g., U.S. Pat. No. 7,427,672); 4′-CH₂—C(H)(CH₃)-2′ (see, e.g., Chattopadhyaya et al., J. Org. Chem., 2009, 74, 118-134); and 4′-CH₂—C(═CH₂)-2′ (and analogs thereof; see, e.g., U.S. Pat. No. 8,278,426). The entire contents of each of the foregoing are hereby incorporated herein by reference.

Additional representative U.S. patents and US patenttent Publications that teach the preparation of locked nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, the entire contents of each of which are hereby incorporated herein by reference.

Any of the foregoing bicyclic nucleosides can be prepared having one or more stereochemical sugar configurations including for example α-L-ribofuranose and β-D-ribofuranose (see WO 99/14226).

An oligonucleotide for use in the methods of the invention can also be modified to include one or more constrained ethyl nucleotides. As used herein, a “constrained ethyl nucleotide” or “cEt” is a locked nucleic acid including a bicyclic sugar moiety including a 4′-CH(CH3)-O-2′ bridge. In one embodiment, a constrained ethyl nucleotide is in the S conformation referred to herein as “S-cEt.”

An oligonucleotide for use in the methods of the invention may also include one or more “conformationally restricted nucleotides” (“CRN”). CRN are nucleotide analogs with a linker connecting the C2′ and C4′ carbons of ribose or the C3 and —C5′ carbons of ribose. CRN lock the ribose ring into a stable conformation and increase the hybridization affinity to mRNA. The linker is of sufficient length to place the oxygen in an optimal position for stability and affinity resulting in less ribose ring puckering.

Representative publications that teach the preparation of certain of the above noted CRN include, but are not limited to, US Patent Publication No. 2013/0190383; and PCT publication WO 2013/036868, the entire contents of each of which are hereby incorporated herein by reference.

In some embodiments, an oligonucleotide for use in the methods of the invention includes one or more monomers that are UNA (unlocked nucleic acid) nucleotides. UNA is unlocked acyclic nucleic acid, wherein any of the bonds of the sugar has been removed, forming an unlocked “sugar” residue. In one example, UNA also encompasses monomer with bonds between C1′-C4′ have been removed (i.e. the covalent carbon-oxygen-carbon bond between the C1′ and C4′ carbons). In another example, the C2′-C3′ bond (i.e. the covalent carbon-carbon bond between the C2′ and C3′ carbons) of the sugar has been removed (see Nuc. Acids Symp. Series, 52, 133-134 (2008) and Fluiter et al., Mol. Biosyst., 2009, 10, 1039 hereby incorporated by reference).

Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

The ribose molecule may also be modified with a cyclopropane ring to produce a tricyclodeoxynucleic acid (tricyclo DNA). The ribose moiety may be substituted for another sugar such as 1,5,-anhydrohexitol, threose to produce a threose nucleoside (TNA), or arabinose to produce an arabino nucleoside. The ribose molecule can also be replaced with non-sugars such as cyclohexene to produce cyclohexene nucleoside or glycol to produce glycol nucleosides.

The ribose molecule can also be replaced with non-sugars such as cyclohexene to produce cyclohexene nucleic acid (CeNA) or glycol to produce glycol nucleic acids (GNA). Potentially stabilizing modifications to the ends of nucleotide molecules can include N-(acetylaminocaproyl)-4-hydroxyprolinol (Hyp-C6-NHAc), N-(caproyl-4-hydroxyprolinol (Hyp-C6), N-(acetyl-4-hydroxyprolinol (Hyp-NHAc), thymidine-2′-O-deoxythymidine (ether), N-(aminocaproyl)-4-hydroxyprolinol (Hyp-C6-amino), 2-docosanoyl-uridine-3″-phosphate, inverted base dT(idT) and others. Disclosure of this modification can be found in PCT Publication No. WO 2011/005861.

Other alternatives chemistries of an oligonucleotide of the invention include a 5′ phosphate or 5′ phosphate mimic, e.g., a 5′-terminal phosphate or phosphate mimic of an oligonucleotide. Suitable phosphate mimics are disclosed in, for example US Patent Publication No. 2012/0157511, the entire contents of which are incorporated herein by reference.

Exemplary oligonucleotides for use in the methods of the invention include sugar-modified nucleosides and may also include DNA or RNA nucleosides. In some embodiments, the oligonucleotide includes sugar-modified nucleosides and DNA nucleosides. Incorporation of alternative nucleosides into the oligonucleotide of the invention may enhance the affinity of the oligonucleotide for the target nucleic acid. In that case, the alternative nucleosides can be referred to as affinity enhancing alternative nucleotides.

In some embodiments, the oligonucleotide includes at least 1 alternative nucleoside, such as at least 2, at least 3, at least 4, at least 5, at least 6, at least 7, at least 8, at least 9, at least 10, at least 11, at least 12, at least 13, at least 14, at least 15 or at least 16 alternative nucleosides. In other embodiments, the oligonucleotides include from 1 to 10 alternative nucleosides, such as from 2 to 9 alternative nucleosides, such as from 3 to 8 alternative nucleosides, such as from 4 to 7 alternative nucleosides, such as 6 or 7 alternative nucleosides. In an embodiment, the oligonucleotide of the invention may include alternatives, which are independently selected from these three types of alternative (alternative sugar moiety, alternative nucleobase, and alternative internucleoside linkage), or a combination thereof. Preferably the oligonucleotide includes one or more nucleosides including alternative sugar moieties, e.g., 2′ sugar alternative nucleosides. In some embodiments, the oligonucleotide of the invention include the one or more 2′ sugar alternative nucleoside independently selected from the group consisting of 2′-O-alkyl-RNA, 2′-O-methyl-RNA, 2′-alkoxy-RNA, 2′-O-methoxyethyl-RNA, 2′-amino-DNA, 2′-fluoro-DNA, ANA, 2′-fluoro-ANA, and BNA (e.g., LNA) nucleosides. In some embodiments, the one or more alternative nucleoside is a BNA.

In some embodiments, at least 1 of the alternative nucleosides is a BNA (e.g., an LNA), such as at least 2, such as at least 3, at least 4, at least 5, at least 6, at least 7, or at least 8 of the alternative nucleosides are BNAs. In a still further embodiment, all the alternative nucleosides are BNAs.

In a further embodiment the oligonucleotide includes at least one alternative internucleoside linkage. In some embodiments, the internucleoside linkages within the contiguous nucleotide sequence are phosphorothioate or boronophosphate internucleoside linkages. In some embodiments, all the internucleotide linkages in the contiguous sequence of the oligonucleotide are phosphorothioate linkages. In some embodiments the phosphorothioate linkages are stereochemically pure phosphorothioate linkages. In some embodiments, the phosphorothioate linkages are Sp phosphorothioate linkages. In other embodiments, the phosphorothioate linkages are Rp phosphorothioate linkages.

In some embodiments, the oligonucleotide for use in the methods of the invention includes at least one alternative nucleoside which is a 2′-O-MOE-RNA, such as 2, 3, 4, 5, 6, 7, 8, 9, or 10 2′-O-MOE-RNA nucleoside units. In some embodiments, the 2′-O-MOE-RNA nucleoside units are connected by phosphorothioate linkages. In some embodiments, at least one of said alternative nucleoside is 2′-fluoro DNA, such as 2, 3, 4, 5, 6, 7, 8, 9, or 10 2′-fluoro-DNA nucleoside units. In some embodiments, the oligonucleotide of the invention includes at least one BNA unit and at least one 2′ substituted alternative nucleoside. In some embodiments of the invention, the oligonucleotide includes both 2′ sugar modified nucleosides and DNA units.

B. Oligonucleotide Conjugated to Ligands

Oligonucleotides for use in the methods of the invention may be chemically linked to one or more ligands, moieties, or conjugates that enhance the activity, cellular distribution, or cellular uptake of the oligonucleotide. Such moieties include but are not limited to lipid moieties such as a cholesterol moiety (Letsinger et al., (1989) Proc. Natl. Acid. Sci. USA, 86: 6553-6556), cholic acid (Manoharan et al., (1994) Biorg. Med. Chem. Let., 4:1053-1060), a thioether, e.g., beryl-S-tritylthiol (Manoharan et al., (1992) Ann. N.Y. Acad. Sci., 660:306-309; Manoharan et al., (1993) Biorg. Med. Chem. Let., 3:2765-2770), a thiocholesterol (Oberhauser et al., (1992) Nucl. Acids Res., 20:533-538), an aliphatic chain, e.g., dodecandiol or undecyl residues (Saison-Behmoaras et al., (1991) EMBO J, 10:1111-1118; Kabanov et al., (1990) FEBS Lett., 259:327-330; Svinarchuk et al., (1993) Biochimie, 75:49-54), a phospholipid, e.g., di-hexadecyl-rac-glycerol or triethyl-ammonium 1,2-di-O-hexadecyl-rac-glycero-3-phosphonate (Manoharan et al., (1995) Tetrahedron Lett., 36:3651-3654; Shea et al., (1990) Nucl. Acids Res., 18:3777-3783), a polyamine or a polyethylene glycol chain (Manoharan et al., (1995) Nucleosides & Nucleotides, 14:969-973), or adamantane acetic acid (Manoharan et al., (1995) Tetrahedron Lett., 36:3651-3654), a palmityl moiety (Mishra et al., (1995) Biochim. Biophys. Acta, 1264:229-237), or an octadecylamine or hexylamino-carbonyloxycholesterol moiety (Crooke et al., (1996) J. Pharmacol. Exp. Ther., 277:923-937).

In one embodiment, a ligand alters the distribution, targeting, or lifetime of an oligonucleotide agent into which it is incorporated. In some embodiments, a ligand provides an enhanced affinity for a selected target, e.g., molecule, cell or cell type, compartment, e.g., a cellular or organ compartment, tissue, organ, or region of the body, as, e.g., compared to a species absent such a ligand.

Ligands can include a naturally occurring substance, such as a protein (e.g., human serum albumin (HSA), low-density lipoprotein (LDL), or globulin); carbohydrate (e.g., a dextran, pullulan, chitin, chitosan, inulin, cyclodextrin, N-acetylglucosamine, N-acetylgalactosamine, or hyaluronic acid); or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolied) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic ionizable lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, vitamin A, biotin, or an RGD peptide or RGD peptide mimetic.

Other examples of ligands include dyes, intercalating agents (e.g. acridines), cross-linkers (e.g. psoralen, mitomycin C), porphyrins (TPPC4, texaphyrin, Sapphyrin), polycyclic aromatic hydrocarbons (e.g., phenazine, dihydrophenazine), artificial endonucleases (e.g. EDTA), lipophilic molecules, e.g., cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a hepatic cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, or multivalent fucose.

The ligand can be a substance, e.g., a drug, which can increase the uptake of the oligonucleotide agent into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin.

In some embodiments, a ligand attached to an oligonucleotide as described herein acts as a pharmacokinetic modulator (PK modulator). PK modulators include lipophiles, bile acids, steroids, phospholipid analogues, peptides, protein binding agents, PEG, vitamins etc. Exemplary PK modulators include, but are not limited to, cholesterol, fatty acids, cholic acid, lithocholic acid, dialkylglycerides, diacylglyceride, phospholipids, sphingolipids, naproxen, ibuprofen, vitamin E, biotin etc. Oligonucleotides that include a number of phosphorothioate linkages are also known to bind to serum protein, thus short oligonucleotides, e.g., oligonucleotides of about 5 bases, 10 bases, 15 bases, or 20 bases, including multiple of phosphorothioate linkages in the backbone are also amenable to the present invention as ligands (e.g. as PK modulating ligands). In addition, aptamers that bind serum components (e.g. serum proteins) are also suitable for use as PK modulating ligands in the embodiments described herein.

Ligand-conjugated oligonucleotides of the invention may be synthesized by the use of an oligonucleotide that bears a pendant reactive functionality, such as that derived from the attachment of a linking molecule onto the oligonucleotide (described below). This reactive oligonucleotide may be reacted directly with commercially-available ligands, ligands that are synthesized bearing any of a variety of protecting groups, or ligands that have a linking moiety attached thereto.

The oligonucleotides used in the conjugates of the present invention may be conveniently and routinely made through the well-known technique of solid-phase synthesis. Equipment for such synthesis is sold by several vendors including, for example, Applied Biosystems (Foster City, Calif.). Any other means for such synthesis known in the art may additionally or alternatively be employed. It is also known to use similar techniques to prepare other oligonucleotides, such as the phosphorothioates and alkylated derivatives.

In the ligand-conjugated oligonucleotides of the present invention, such as the ligand-molecule bearing sequence-specific linked nucleosides of the present invention, the oligonucleotides and oligonucleosides may be assembled on a suitable DNA synthesizer utilizing standard nucleotide or nucleoside precursors, or nucleotide or nucleoside conjugate precursors that already bear the linking moiety, ligand-nucleotide or nucleoside-conjugate precursors that already bear the ligand molecule, or non-nucleoside ligand-bearing building blocks.

When using nucleotide-conjugate precursors that already bear a linking moiety, the synthesis of the sequence-specific linked nucleosides is typically completed, and the ligand molecule is then reacted with the linking moiety to form the ligand-conjugated oligonucleotide. In some embodiments, the oligonucleotides or linked nucleosides of the present invention are synthesized by an automated synthesizer using phosphoramidites derived from ligand-nucleoside conjugates in addition to the standard phosphoramidites and non-standard phosphoramidites that are commercially available and routinely used in oligonucleotide synthesis.

i. Lipid Conjugates

In one embodiment, the ligand or conjugate is a lipid or lipid-based molecule. Such a lipid or lipid-based molecule preferably binds a serum protein, e.g., human serum albumin (HSA). An HSA binding ligand allows for distribution of the conjugate to a target tissue, e.g., a non-kidney target tissue of the body. For example, the target tissue can be the liver, including parenchymal cells of the liver. Other molecules that can bind HSA can also be used as ligands. For example, neproxin or aspirin can be used. A lipid or lipid-based ligand can (a) increase resistance to degradation of the conjugate, (b) increase targeting or transport into a target cell or cell membrane, and/or (c) can be used to adjust binding to a serum protein, e.g., HSA.

A lipid-based ligand can be used to inhibit, e.g., control the binding of the conjugate to a target tissue. For example, a lipid or lipid-based ligand that binds to HSA more strongly will be less likely to be targeted to the kidney and therefore less likely to be cleared from the body. A lipid or lipid-based ligand that binds to HSA less strongly can be used to target the conjugate to the kidney.

In another aspect, the ligand is a moiety, e.g., a vitamin, which is taken up by a target cell, e.g., a proliferating cell. Exemplary vitamins include vitamin A, E, and K.

ii. Cell Permeation Agents

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the oligonucleotide, such as by enhancing cellular recognition and absorption. The peptide or peptidomimetic moiety can be about 5-50 amino acids long, e.g., about 5, 10, 15, 20, 25, 30, 35, 40, 45, or 50 amino acids long.

A peptide or peptidomimetic can be, for example, a cell permeation peptide, cationic peptide, amphipathic peptide, or hydrophobic peptide (e.g., consisting primarily of Tyr, Trp, or Phe). The peptide moiety can be a dendrimer peptide, constrained peptide or crosslinked peptide. In another alternative, the peptide moiety can include a hydrophobic membrane translocation sequence (MTS). An exemplary hydrophobic MTS-containing peptide is RFGF having the amino acid sequence AAVALLPAVLLALLAP (SEQ ID NO. 51). An RFGF analogue (e.g., amino acid sequence AALLPVLLAAP (SEQ ID NO. 52) containing a hydrophobic MTS can also be a targeting moiety. The peptide moiety can be a “delivery” peptide, which can carry large polar molecules including peptides, oligonucleotides, and protein across cell membranes. For example, sequences from the HIV Tat protein (GRKKRRQRRRPPQ; SEQ ID NO. 53) and the Drosophila Antennapedia protein (RQIKIWFQNRRMKWKK; SEQ ID NO. 54) have been found to be capable of functioning as delivery peptides. A peptide or peptidomimetic can be encoded by a random sequence of DNA, such as a peptide identified from a phage-display library, or one-bead-one-compound (OBOC) combinatorial library (Lam et al., Nature, 354:82-84, 1991). Examples of a peptide or peptidomimetic tethered to an oligonucleotide agent via an incorporated monomer unit for cell targeting purposes is an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

An RGD peptide for use in the compositions and methods of the invention may be linear or cyclic, and may be modified, e.g., glycosylated or methylated, to facilitate targeting to a specific tissue(s). RGD-containing peptides and peptidomimetics may include D-amino acids, as well as synthetic RGD mimics. In addition to RGD, one can use other moieties that target the integrin ligand. Some conjugates of this ligand target PECAM-1 or VEGF.

A cell permeation peptide is capable of permeating a cell, e.g., a microbial cell, such as a bacterial or fungal cell, or a mammalian cell, such as a human cell. A microbial cell-permeating peptide can be, for example, an α-helical linear peptide (e.g., LL-37 or Ceropin P1), a disulfide bond-containing peptide (e.g., α-defensin, β-defensin, or bactenecin), or a peptide containing only one or two dominating amino acids (e.g., PR-39 or indolicidin). A cell permeation peptide can also include a nuclear localization signal (NLS). For example, a cell permeation peptide can be a bipartite amphipathic peptide, such as MPG, which is derived from the fusion peptide domain of HIV-1 gp41 and the NLS of SV40 large T antigen (Simeoni et al., Nucl. Acids Res. 31:2717-2724, 2003).

iii. Carbohydrate Conjugates

In some embodiments of the compositions and methods of the invention, an oligonucleotide further includes a carbohydrate. The carbohydrate conjugated oligonucleotide is advantageous for the in vivo delivery of nucleic acids, as well as compositions suitable for in vivo therapeutic use, as described herein. As used herein, “carbohydrate” refers to a compound which is either a carbohydrate per se made up of one or more monosaccharide units having at least 6 carbon atoms (which can be linear, branched or cyclic) with an oxygen, nitrogen or sulfur atom bonded to each carbon atom; or a compound having as a part thereof a carbohydrate moiety made up of one or more monosaccharide units each having at least six carbon atoms (which can be linear, branched or cyclic), with an oxygen, nitrogen or sulfur atom bonded to each carbon atom. Representative carbohydrates include the sugars (mono-, di-, tri- and oligosaccharides containing from about 4, 5, 6, 7, 8, or 9 monosaccharide units), and polysaccharides such as starches, glycogen, cellulose and polysaccharide gums. Specific monosaccharides include C5 and above (e.g., C5, C6, C7, or C8) sugars; di- and trisaccharides include sugars having two or three monosaccharide units (e.g., C5, C6, C7, or C8).

In one embodiment, a carbohydrate conjugate for use in the compositions and methods of the invention is a monosaccharide.

In some embodiments, the carbohydrate conjugate further includes one or more additional ligands as described above, such as, but not limited to, a PK modulator and/or a cell permeation peptide.

Additional carbohydrate conjugates (and linkers) suitable for use in the present invention include those described in PCT Publication Nos. WO 2014/179620 and WO 2014/179627, the entire contents of each of which are incorporated herein by reference.

iv. Linkers

In some embodiments, the conjugate or ligand described herein can be attached to an oligonucleotide with various linkers that can be cleavable or non-cleavable.

Linkers typically include a direct bond or an atom such as oxygen or sulfur, a unit such as NR⁸, C(O), C(O)NH, SO, SO₂, SO₂NH or a chain of atoms, such as, but not limited to, substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, which one or more methylenes can be interrupted or terminated by O, S, S(O), SO₂, N(R⁸), C(O), substituted or unsubstituted aryl, substituted or unsubstituted heteroaryl, substituted or unsubstituted heterocyclic; where R⁸ is hydrogen, acyl, aliphatic or substituted aliphatic. In one embodiment, the linker is between about 1-24 atoms, 2-24, 3-24, 4-24, 5-24, 6-24, 6-18, 7-18, 8-18 atoms, 7-17, 8-17, 6-16, 7-17, or 8-16 atoms.

A cleavable linking group is one which is sufficiently stable outside the cell, but which upon entry into a target cell is cleaved to release the two parts the linker is holding together. In a preferred embodiment, the cleavable linking group is cleaved at least about 10 times, 20, times, times, 40 times, 50 times, 60 times, 70 times, 80 times, 90 times, or more, or at least about 100 times faster in a target cell or under a first reference condition (which can, e.g., be selected to mimic or represent intracellular conditions) than in the blood of a subject, or under a second reference condition (which can, e.g., be selected to mimic or represent conditions found in the blood or serum).

Cleavable linking groups are susceptible to cleavage agents, e.g., pH, redox potential, or the presence of degradative molecules. Generally, cleavage agents are more prevalent or found at higher levels or activities inside cells than in serum or blood. Examples of such degradative agents include: redox agents which are selective for particular substrates or which have no substrate specificity, including, e.g., oxidative or reductive enzymes or reductive agents such as mercaptans, present in cells, that can degrade a redox cleavable linking group by reduction; esterases; endosomes or agents that can create an acidic environment, e.g., those that result in a pH of five or lower; enzymes that can hydrolyze or degrade an acid cleavable linking group by acting as a general acid, peptidases (which can be substrate specific), and phosphatases.

A cleavable linkage group, such as a disulfide bond can be susceptible to pH. The pH of human serum is 7.4, while the average intracellular pH is slightly lower, ranging from about 7.1-7.3. Endosomes have a more acidic pH, in the range of 5.5-6.0, and lysosomes have an even more acidic pH at around 5.0. Some linkers will have a cleavable linking group that is cleaved at a preferred pH, thereby releasing a cationic lipid from the ligand inside the cell, or into the desired compartment of the cell.

A linker can include a cleavable linking group that is cleavable by a particular enzyme. The type of cleavable linking group incorporated into a linker can depend on the cell to be targeted. For example, a liver-targeting ligand can be linked to a cationic lipid through a linker that includes an ester group. Liver cells are rich in esterases, and therefore the linker will be cleaved more efficiently in liver cells than in cell types that are not esterase-rich. Other cell-types rich in esterases include cells of the lung, renal cortex, and testis.

Linkers that contain peptide bonds can be used when targeting cell types rich in peptidases, such as liver cells and synoviocytes.

In general, the suitability of a candidate cleavable linking group can be evaluated by testing the ability of a degradative agent (or condition) to cleave the candidate linking group. It will also be desirable to also test the candidate cleavable linking group for the ability to resist cleavage in the blood or when in contact with other non-target tissues. Thus, one can determine the relative susceptibility to cleavage between a first and a second condition, where the first is selected to be indicative of cleavage in a target cell and the second is selected to be indicative of cleavage in other tissues or biological fluids, e.g., blood or serum. The evaluations can be carried out in cell free systems, in cells, in cell culture, in organ or tissue culture, or in whole animals. It can be useful to make initial evaluations in cell-free or culture conditions and to confirm by further evaluations in whole animals. In preferred embodiments, useful candidate compounds are cleaved at least about 2, 4, 10, 20, 30, 40, 50, 60, 70, 80, 90, or about 100 times faster in the cell (or under in vitro conditions selected to mimic intracellular conditions) as compared to blood or serum (or under in vitro conditions selected to mimic extracellular conditions).

a. Redox Cleavable Linking Groups

In one embodiment, a cleavable linking group is a redox cleavable linking group that is cleaved upon reduction or oxidation. An example of reductively cleavable linking group is a disulphide linking group (—S—S—). To determine if a candidate cleavable linking group is a suitable “reductively cleavable linking group,” or for example is suitable for use with a particular oligonucleotide moiety and particular targeting agent one can look to methods described herein. For example, a candidate can be evaluated by incubation with dithiothreitol (DTT), or other reducing agent using reagents know in the art, which mimic the rate of cleavage which would be observed in a cell, e.g., a target cell. The candidates can also be evaluated under conditions which are selected to mimic blood or serum conditions. In one embodiment, candidate compounds are cleaved by at most about 10% in the blood. In other embodiments, useful candidate compounds are degraded at least about 2, 4, 10, 20, 30, 40, 50, 60, 70, 80, 90, or about 100 times faster in the cell (or under in vitro conditions selected to mimic intracellular conditions) as compared to blood (or under in vitro conditions selected to mimic extracellular conditions). The rate of cleavage of candidate compounds can be determined using standard enzyme kinetics assays under conditions chosen to mimic intracellular media and compared to conditions chosen to mimic extracellular media.

b. Phosphate-Based Cleavable Linking Groups

In another embodiment, a cleavable linker includes a phosphate-based cleavable linking group. A phosphate-based cleavable linking group is cleaved by agents that degrade or hydrolyze the phosphate group. An example of an agent that cleaves phosphate groups in cells are enzymes such as phosphatases in cells. Examples of phosphate-based linking groups are —O—P(O)(OR^{k})—O—, —O—P(S)(OR^{k})—O—, —O—P(S)(SR^{k})—O—, —S—P(O)(OR^{k})—O—, —O—P(O)(OR^{k})—S—, —S—P(O)(OR^{k})—S—, —O—P(S)(OR^{k})—S—, —S—P(S)(OR^{k})—O—, —O—P(O)(R^{k})—O—, —O—P(S)(R^{k})—O—, —S—P(O)(R^{k})—O—, —S—P(S)(R^{k})—O—, —S—P(O)(R^{k})—S—, —O—P(S)(R^{k})—S—. These candidates can be evaluated using methods analogous to those described above.

c. Acid Cleavable Linking Groups

In another embodiment, a cleavable linker includes an acid cleavable linking group. An acid cleavable linking group is a linking group that is cleaved under acidic conditions. In preferred embodiments acid cleavable linking groups are cleaved in an acidic environment with a pH of about 6.5 or lower (e.g., about 6.0, 5.75, 5.5, 5.25, 5.0, or lower), or by agents such as enzymes that can act as a general acid. In a cell, specific low pH organelles, such as endosomes and lysosomes can provide a cleaving environment for acid cleavable linking groups. Examples of acid cleavable linking groups include but are not limited to hydrazones, esters, and esters of amino acids. Acid cleavable groups can have the general formula —C═NN—, C(O)O, or —OC(O). A preferred embodiment is when the carbon attached to the oxygen of the ester (the alkoxy group) is an aryl group, substituted alkyl group, or tertiary alkyl group such as dimethyl pentyl or t-butyl. These candidates can be evaluated using methods analogous to those described above.

d. Ester-Based Linking Groups

In another embodiment, a cleavable linker includes an ester-based cleavable linking group. An ester-based cleavable linking group is cleaved by enzymes such as esterases and amidases in cells. Examples of ester-based cleavable linking groups include but are not limited to esters of alkylene, alkenylene and alkynylene groups. Ester cleavable linking groups have the general formula —C(O)O—, or —OC(O)—. These candidates can be evaluated using methods analogous to those described above.

e. Peptide-Based Cleaving Groups

In yet another embodiment, a cleavable linker includes a peptide-based cleavable linking group. A peptide-based cleavable linking group is cleaved by enzymes such as peptidases and proteases in cells. Peptide-based cleavable linking groups are peptide bonds formed between amino acids to yield oligopeptides (e.g., dipeptides, tripeptides etc.) and polypeptides. Peptide-based cleavable groups do not include the amide group (—C(O)NH—). The amide group can be formed between any alkylene, alkenylene, or alkynelene. A peptide bond is a special type of amide bond formed between amino acids to yield peptides and proteins. The peptide-based cleavage group is generally limited to the peptide bond (i.e., the amide bond) formed between amino acids yielding peptides and proteins and does not include the entire amide functional group. Peptide-based cleavable linking groups have the general formula —NHCHRAC(O)NHCHRBC(O)—, where RA and RB are the R groups of the two adjacent amino acids. These candidates can be evaluated using methods analogous to those described above.

In one embodiment, an oligonucleotide of the invention is conjugated to a carbohydrate through a linker. Linkers include bivalent and trivalent branched linker groups. Exemplary oligonucleotide carbohydrate conjugates with linkers of the compositions and methods of the invention include, but are not limited to, those described in formulas 24-35 of PCT Publication No. WO 2018/195165.

Representative U.S. patents that teach the preparation of oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941; 6,294,664; 6,320,017; 6,576,752; 6,783,931; 6,900,297; 7,037,646; 8,106,022, the entire contents of each of which are hereby incorporated herein by reference.

In certain instances, the nucleotides of an oligonucleotide can be modified by a non-ligand group. A number of non-ligand molecules have been conjugated to oligonucleotides in order to enhance the activity, cellular distribution, or cellular uptake of the oligonucleotide, and procedures for performing such conjugations are available in the scientific literature. Such non-ligand moieties have included lipid moieties, such as cholesterol (Kubo, T. et al., Biochem. Biophys. Res. Comm, 2007, 365(1):54-61; Letsinger et al., Proc. Natl. Acad. Sci. USA, 1989, 86:6553), cholic acid (Manoharan et al., Bioorg. Med. Chem. Lett., 1994, 4:1053), a thioether, e.g., hexyl-S-tritylthiol (Manoharan et al., Ann. N.Y. Acad. Sci., 1992, 660:306; Manoharan et al., Bioorg. Med. Chem. Let., 1993, 3:2765), a thiocholesterol (Oberhauser et al., Nucl. Acids Res., 1992, 20:533), an aliphatic chain, e.g., dodecandiol or undecyl residues (Saison-Behmoaras et al., EMBO J., 1991, 10:111; Kabanov et al., FEBS Lett., 1990, 259:327; Svinarchuk et al., Biochimie, 1993, 75:49), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36:3651; Shea et al., Nucl. Acids Res., 1990, 18:3777), a polyamine or a polyethylene glycol chain (Manoharan et al., Nucleosides & Nucleotides, 1995, 14:969), or adamantane acetic acid (Manoharan et al., Tetrahedron Lett., 1995, 36:3651), a palmityl moiety (Mishra et al., Biochim. Biophys. Acta, 1995, 1264:229), or an octadecylamine or hexylamino-carbonyl-oxycholesterol moiety (Crooke et al., J. Pharmacol. Exp. Ther., 1996, 277:923). Representative United States patents that teach the preparation of such oligonucleotide conjugates have been listed above. Typical conjugation protocols involve the synthesis of an oligonucleotide bearing an amino linker at one or more positions of the sequence. The amino group is then reacted with the molecule being conjugated using appropriate coupling or activating reagents. The conjugation reaction can be performed either with the oligonucleotide still bound to the solid support or following cleavage of the oligonucleotide, in solution phase. Purification of the oligonucleotide conjugate by HPLC typically affords the pure conjugate.

### IV. Pharmaceutical Compositions

The present disclosure also includes pharmaceutical compositions and formulations which include the oligonucleotides of the disclosure. In one embodiment, provided herein are pharmaceutical compositions containing an oligonucleotide, e.g., a guide oligonucleotide, as described herein, and a pharmaceutically acceptable carrier. The pharmaceutical compositions containing the oligonucleotide are useful for treating a subject who would benefit from editing a target gene, e.g., a MECP2 polynucleotide with a SNP associated with Rett syndrome.

The pharmaceutical compositions of the present disclosure can be administered in a number of ways depending upon whether local or systemic treatment is desired and upon the area to be treated. Administration can be oral, parental, topical (e.g., by a transdermal patch), intranasal, intratracheal, epidermal and transdermal.

Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration. Parenteral administration may be by continuous infusion over a selected period of time.

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the oligonucleotides featured in the disclosure are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). Oligonucleotides featured in the disclosure can be encapsulated within liposomes or can form complexes thereto, in particular to cationic liposomes. Alternatively, Oligonucleotides can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-20 alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

Compositions and formulations for parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Useful solutions for oral or parenteral administration can be prepared by any of the methods well known in the pharmaceutical art, described, for example; in Remington's Pharmaceutical Sciences, 18th ed. (Mack Publishing Company, 1990). The parenteral preparation can be enclosed in ampoules, disposable syringes or multiple dose vials made of glass or plastic. Formulations also can include, for example, polyalkylene glycols such as polyethylene glycol, oils of vegetable origin, and hydrogenated naphthalenes. Other potentially useful parenteral carriers for these drugs include ethylene-vinyl acetate copolymer particles, osmotic pumps, implantable infusion systems, and liposomes.

Formulations of the present disclosure suitable for oral administration may be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug may also be administered in the form of a bolus, electuary or paste. A tablet may be made by compressing or molding the drug optionally with one or more accessory ingredients. Compressed tablets may be prepared by compressing, in a suitable machine, the drug in a free-flowing form such as a powder or granules, optionally mixed by a binder, lubricant, inert diluent, surface active or dispersing agent. Molded tablets may be made by molding; in a suitable machine; a mixture of the powdered drug and suitable carrier moistened with an inert liquid diluent.

Oral compositions generally include an inert diluent or an edible carrier. For the purpose of oral therapeutic administration, the active compound can be incorporated with excipients. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water; ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions; methods of preparation include vacuum drying and freeze-drying which yields a powder of the active ingredient plus any additional desired ingredient from a previously sterile-filtered solution thereof.

Formulations suitable for intra-articular administration may be in the form of a sterile aqueous preparation of the drug that may be in microcrystal line form, for example, in the form of an aqueous microcrystalline suspension. Liposomal formulations or biodegradable polymer systems may also be used to present the drug for both intra-articular and ophthalmic administration.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents and bile salts. Transmucosal administration can be accomplished through the use of nasal sprays or suppositories. For transdermal administration, the active compounds typically are formulated into ointments, salves, gels, or creams as generally known in the art.

The active compounds may be prepared with carriers that will protect the compound against rapid elimination from the body, such as a controlled release formulation, including implants and microencapsulated delivery systems. Biodegradable, biocompatible polymers can be used; such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intravenous bag).

Where the active compound is to be used as part of a transplant procedure, it can be provided to the living tissue or organ to be transplanted prior to removal of tissue or organ from the donor. The compound can be provided to the donor host. Alternatively, or in addition, once removed from the donor, the organ or living tissue can be placed in a preservation solution containing the active compound. In all cases, the active compound can be administered directly to the desired tissue, as by injection to the tissue, or it can be provided systemically, either by oral or parenteral administration, using any of the methods and formulations described herein and/or known in the art. Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure can be formulated into any of many possible dosage forms such as, but not limited to, tablets, capsules, gel capsules, liquid syrups, soft gels, suppositories, and enemas. The compositions of the present disclosure can also be formulated as suspensions in aqueous, non-aqueous or mixed media. Aqueous suspensions can further contain substances which increase the viscosity of the suspension including, for example, sodium carboxymethylcellulose, sorbitol or dextran. The suspension can also contain stabilizers.

The compositions of the present disclosure can also be prepared and formulated in additional formulations, such as emulsions or microemulsions, or be incorporated into a particle, e.g., a microparticle, which can be produced by spray-drying, or other methods including lyophilization, evaporation, fluid bed drying, vacuum drying, or a combination of these techniques. Penetration enhancers, e.g., surfactants, fatty acids, bile salts, chelating agents, and non-chelating non-surfactants, may be added in order to effect the efficient delivery of the compositions of the present disclosure, e.g., the delivery of the oligonucleotides, to the subject. Agents that enhance uptake of oligonucleotide agents at the cellular level can also be added to the pharmaceutical and other compositions of the present disclosure, such as, cationic lipids, e.g., lipofectin, cationic glycerol derivatives, and polycationic molecules, e.g., polylysine.

The pharmaceutical composition of the present disclosure may also include a pharmaceutical carrier or excipient. A pharmaceutical carrier or excipient is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used. Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Toxicity and therapeutic efficacy of the compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). Compounds that exhibit high therapeutic indices are preferred. The data obtained from cell culture assays and animal studies can be used in formulating a range of dosage for use in humans.

The dosage of the compositions (e.g., a composition including an oligonucleotide) described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine whether to administer the composition and tailor the appropriate dosage and/or therapeutic regimen of treatment with the composition based on the above factors. The compositions described herein may be administered initially in a suitable dosage that may be adjusted as required, depending on the clinical response. In some embodiments, the dosage of a composition (e.g., a composition including an oligonucleotide) is a prophylactically or a therapeutically effective amount. In some embodiments, treatment of a subject with a therapeutically effective amount of a composition can include a single treatment or a series of treatments. In addition, it is to be understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

The pharmaceutical compositions of the disclosure may be administered in dosages sufficient to edit a target gene, e.g., a MECP2 polynucleotide, and/or treat Rett syndrome. In therapeutic use for treating, preventing, or combating, Rett syndrome in subjects, the compounds or pharmaceutical compositions thereof will be administered orally or parenterally at a dosage to obtain and maintain a concentration, that is, an amount, or blood-level or tissue level of active component in the animal undergoing treatment which will be effective. The term “effective amount” is understood to mean that the compound of the disclosure is present in or on the recipient in an amount sufficient to elicit biological activity. Generally, an effective amount of dosage of active component will be in the range of from about 1 μg/kg to about 100 mg/kg, preferably from about 10 μg/kg to about 10 mg/kg, more preferably from about 100 μg/kg to about 1 mg/kg of body weight per day.

### V. Kits

In certain aspects, the instant disclosure provides kits that include a pharmaceutical formulation including an oligonucleotide agent capable of effecting an adenosine deaminase acting on RNA (ADAR)-mediated adenosine to inosine alteration of a SNP associated with a disease, e.g., Rett syndrome, and a package insert with instructions to perform any of the methods described herein.

In some embodiments, the kits include instructions for using the kit to edit a MECP2 polynucleotide comprising a SNP associated with Rett syndrome. In other embodiments, the kits include instructions for using the kit to edit a MECP2 polynucleotide comprising a SNP associated with Rett syndrome and to treat Rett syndrome. The instructions will generally include information about the use of the kit for editing nucleic acid molecules. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters.

In some embodiments, the kit includes a pharmaceutical formulation including an oligonucleotide agent capable of effecting an ADAR-mediated adenosine to inosine alteration of a SNP associated with a disease, e.g., Rett syndrome, an additional therapeutic agent, and a package insert with instructions to perform any of the methods described herein.

The kit may be packaged in a number of different configurations such as one or more containers in a single box. The different components can be combined, e.g., according to instructions provided with the kit. The components can be combined according to a method described herein, e.g., to prepare and administer a pharmaceutical composition.

In some embodiments, the kit can comprise one or more containers with appropriate positive and negative controls or control samples, to be used as standard(s) for detection, calibration, or normalization.

The kit can further comprise a second container comprising a pharmaceutically-acceptable buffer, such as (sterile) phosphate-buffered saline, Ringer's solution, or dextrose solution; and other suitable additives such as penetration enhancers, carrier compounds and other pharmaceutically acceptable carriers or excipients, as described herein. It can further include other materials desirable from a commercial and user standpoint, including other buffers, diluents, filters, and package inserts with instructions for use. The kit can also include a drug delivery system such as liposomes, micelles, nanoparticles, and microspheres, as described herein. The kit can further include a delivery device, e.g., for delivery to the central nervous system, such as needles, syringes, pumps, and package inserts with instructions for use.

This invention is further illustrated by the following examples which should not be construed as limiting. The entire contents of all references, patents and published patent applications cited throughout this application, as well as the Figures and the Sequence Listing, are hereby incorporated herein by reference.

### EXAMPLES

### Example 1. Reversing an Amino Acid Substitution Mutation R255X in the MECP2 Transcript by Targeted A to I Editing

Guide oligonucleotides were chemically synthesized on an automated RNA/DNA synthesizer using standard β-cyanoethylphosphoramidite chemistry and a universal solid support such as controlled pore glass (CPG). 5-O-DMT-3-phosphoramidite RNA, 2′-O-methyl-RNA and DNA monomers, i.e., A, C, G, U, and T, were purchased from commercial sources. All oligonucleotides were synthesized by BioSpring GmbH (Frankfurt, Germany) at a 200 nmol scale. After synthesis, oligonucleotides were cleaved from the solid support, deprotected, and purified by a HPLC system using standard protocols. Oligonucleotides were desalted, dialyzed, and lyophilized. The purity of each lyophilized oligo was >90% as determined by analytical reversed-phase HPLC. The sequence integrity of the oligonucleotides was determined by ESI-MS.

Human ADAR2 sequence (NM_001112.4; SEQ ID NO: 55), human ADAR1p110 (NM_001111.5; SEQ ID NO: 56) and human MECP2 R255X (NM_004992; SEQ ID NO: 58) sequences (ORF only), were cloned into pcDNA3.1 plasmid under the control of the CMV promoter using BamHI and XbaI restriction sites (Quintara Bio, Berkeley, Calif.) and the correct insert was sequence verified. For the human MECP2 R255X construct, recombinant HA-tag is placed in-frame at the N-terminus of the coding sequence. The plasmids will henceforth be denoted as ADAR2/pcDNA3.1, ADAR1p110/pcDNA3.1, or MECP2/pcDNA3.1. For editing experiments, 2 μg of ADAR2/pcDNA3.1 or ADAR1p110/pcDNA3.1 plasmid and 10 μg of MECP2/pcDNA3.1 plasmid were transfected into 5×10⁶ HEK293T cells (ATCC) using 25 μL of Lipofectamine 3000 and 24 μL of P3000 (Life Technologies) per 10 cm dish. After 4 hours, the culture media was replenished with fresh warmed media (DMEM High Glucose; Life Technologies). 12-16 hours after transfection, the transfected HEK293T cells were transfected with guide oligonucleotides such that the final concentration in each well was 100 nM. All transfections were carried out with Lipofectamine 3000 (0.4 μL/per well) in a 96-well format according to the manufacturer's instructions. 12-16 hours after the second transfection, the cells were washed once with ice cold PBS and total mRNA isolation was performed using Dyna Beads mRNA Direct Kit (Life Technologies) adapted for KingFisher Flex Purification (Life Technologies) according to the manufacturer's instructions. The samples were treated with TURBO DNase (Life Technologies) prior to elution. The resultant isolated mRNA was used for cDNA synthesis using SuperScript IV Vilo according to the manufacturer's instructions (Life Technologies). One μl of the cDNA was used as template for PCR (Platinum II Hot-Start PCR Master Mix; Life Technologies) using gene specific primers to generate an amplicon for Sanger sequencing (Table 4). Sanger sequencing was performed by Quintara Biosciences (Berkeley, Calif.). Adenosine to inosine editing yields were quantified by measuring the peak height of adenosine and guanosine and dividing the guanosine peak height by the total peak height measurements of adenosine and guanosine combined.

TABLE 4
Primers Used for RT-PCR

| Name | Sequence (5′ to 3′) | SEQ ID NO. |
|---|---|---|
| MECP2 | AGATGCCTTTCCAAGCATCG | 60 |
| R2554X Forward | CC | |
| MECP2 | TGGACACCAGCAGGGGCTTC | 61 |
| R2554X Reverse | | |

ADAR mediated editing of exemplary modified guide oligonucleotides targeting human MECP2 R255X are described in Table 5.

Abbreviations of various modifications are shown in the table below. For example, A, C, G and U are ribonucleosides; dA, dC, dG and dT are deoxyribonucleosides; mA, mC, mG and mU are 2′-O-methyl ribonucleosides; fA, fC, fG and fU are 2′-fluoroarabinonucleosides (FANA). The DNA triplet (dTdCdG) and the FANA triplet (fUfCfG) are indicated in bold. Mismatches are indicated in bold and underline. GluR refers to the natural ADAR substrate, GluR2 receptor pre-mRNA or longer RNA sequences that form a hairpin structure similar to GluR2.

| Modification | Abbreviation |
|---|---|
| RNA | rN |
| DNA | dN |
| 2′-O-Methyl(2′-OMe) | mN |
| 2′-F-arabinose (FANA) | fN |
| 2′-F-RNA | FN |
| 2′-O-Methoxyethyl (MOE) | MN |
| Locked Nucleic Acids (LNA) | LN |
| Abasic | ds |
| RNA-Abasic | rs |
| 2′-OMe-Abasic | mS |
| 5-Methyl-rC | rmC |
| 5-Methyl-dC | dmC |
| 5-Methyl-2′-OMe-C | mmC |
| (S)-Glycol Nucleic Acid (S-GNA) | sgN |
| (R)-Glycol Nucleic Acid (R-GNA) | rgN |
| DNA α-anomer | αN |
| Arabinose | aN |
| 2′-OMe-Arabinose | amN |
| Flexible Nucleic Acid (FNA) | fxN |
| Serineol Nucleic Acid | sN |
| β-D-homoDNA | hdN |
| β-D-HNA | hN |
| Phosphorothioate internucleoside linkage | * |

TABLE 5
Guide Oligonucleotides Targeting Human MECP2 (R255X)

| | | | | | | ADAR1 | ADAR1 |
|---|---|---|---|---|---|---|---|
| SEQ | | | | ADAR2 | ADAR2 | p1110 | p110 |
| ID | Oligo | | | % | St. | % | St. |
| NO. | ID | Length | Guide RNA Sequences | editing | Dev. | Editing | Dev. |
| | | | Human MeCP2 R255X- | | | | |
| | | | GluR-gRNA | | | | |
| | | | (R255X → R255W) | | | | |
| 62 | KB- | 63 | 5′- | 12.3 | 17.5 | 0.0 | 0.0 |
| | 021- | | mG*mU*mG*mG*mAmAmUmAmGmUmA | | | | |
| | 245 | | mUmAmAmCmAmAmUmAmUmGmCmUmA | | | | |
| | | | mAmAmUmGmUmUmUmUmUmAmUmAmG | | | | |
| | | | mUmAmUmCmCmCmAmC- | | | | |
| | | | mCmAmGmCmUmUdTdCdCmAmCmUmU | | | | |
| | | | mC*mC*mU*mG*mC-3′ | | | | |
| 63 | KB- | 65 | 5′- | 27.4 | 2.1 | 0.0 | 0.0 |
| | 021- | | mG*mU*mG*mG*mAmAmUmAmGmUmA | | | | |
| | 246 | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mCmAmGmCmUmUdTdCdCmAmCmUmU | | | | |
| | | | mCmCmUdG*mC*mC*mG-3′ | | | | |
| 64 | KB- | 63 | 5′- | 0.0 | 0.0 | 0.0 | 0.0 |
| | 021- | | mG*mG*mU*mG*mAmAmGmAmGmGmA | | | | |
| | 247 | | mGmAmAmCmAmAmUmAmUmGmCmUmA | | | | |
| | | | mAmAmUmGmUmUmCmUmUmCmUmCmG | | | | |
| | | | mUmCmUmCmCmAmCmC- | | | | |
| | | | mCmAmGmCmUmUdTdCdCmAmCmUmU | | | | |
| | | | mC*mC*mU*mG*mC-3′ | | | | |
| 65 | KB- | 63 | 5′- | 13.9 | 1.7 | 0.0 | 0.0 |
| | 021- | | mG*mG*mU*mG*mUmCmGmAmGmAmA | | | | |
| | 248 | | mGmAmGmGmAmGmAmAmCmAmAmUmA | | | | |
| | | | mUmGmCmUmAmAmAmUmGmUmUmGmU | | | | |
| | | | mUmCmUmCmGmUmCmUmCmCmUmCmG | | | | |
| | | | mAmCmAmCmC- | | | | |
| | | | mCmAmGmCmUmUdTdCdCmAmCmUmU | | | | |
| | | | mC*mC*mU*mG*mC-3′ | | | | |
| | | | Human MeCP2 R255X- | | | | |
| | | | Antisense-gRNA | | | | |
| | | | (R255X → R255W) | | | | |
| 66 | KB- | 50 | 5′- | 0.0 | 0.0 | 0.0 | 0.0 |
| | 021- | | mU*mU*mU*mC*mUmUmGmGmGmAmA | | | | |
| | 249 | | mUmGmGmCmCmUmGmAmGmGmGmUmC | | | | |
| | | | mGmGmCmCmUmCmAmGmCmUmUdTdC | | | | |
| | | | dCmAmCmUmUmCmCmUmG*mC*mC* | | | | |
| | | | mG*mG-3′ | | | | |
| 67 | KB- | 50 | 5′- | 30.5 | 15.5 | 0.0 | 0.0 |
| | 021- | | mU*mU*mU*mC*mUmUmGmGmGmAmA | | | | |
| | 250 | | mUmGmGmCmCmUmGmAmGmGmGmUmC | | | | |
| | | | mGmGmCmCmUmCmAmGmCmUmUmUdC | | | | |
| | | | dCmAmCmUmUmCmCmUmG*mC*mC* | | | | |
| | | | mG*mG-3′ | | | | |
| 68 | KB- | 50 | 5′- | 0.0 | 0.0 | 11.8 | 0.0 |
| | 021- | | mU*mU*mU*mC*mUmUmGmGmGmAmA | | | | |
| | 251 | | mUmGmGmCmCmUmGmAmGmGmGmUmC | | | | |
| | | | mGmGmCmCmUmCmAmGmCmUmUdTdC | | | | |
| | | | mCmAmCmUmUmCmCmUmG*mC*mC* | | | | |
| | | | mG*mG-3′ | | | | |
| 69 | KB- | 50 | 5′- | 17.9 | 0.9 | 0.0 | 0.0 |
| | 021- | | mU*mU*mU*mC*mUmUmGmGmGmAmA | | | | |
| | 252 | | mUmGmGmCmCmUmGmAmGmAmGmUmC | | | | |
| | | | mAmGmCmCmUmCmAmGmCmUmUdTdC | | | | |
| | | | dCmAmCmUmUmCmCmUmG*mC*mC* | | | | |
| | | | mG*mG-3′ | | | | |
| 70 | KB- | 50 | 5′- | 0.0 | 0.0 | 0.0 | 0.0 |
| | 021- | | mU*mU*mU*mC*mUmUmGmGmGmAmA | | | | |
| | 253 | | mUmGmGmCmCmUmGmAmGmAmGmUmC | | | | |
| | | | mAmGmCmCmUmCmAmGmCmUmUdGdC | | | | |
| | | | dCmAmCmUmUmCmCmUmG*mC*mC* | | | | |
| | | | mG*mG-3′ | | | | |
| 71 | KB- | 50 | 5′- | 13.2 | 18.6 | 11.8 | 10.3 |
| | 021- | | mU*mU*mU*mC*mUmUmGmGmGmAmA | | | | |
| | 254 | | mUmGmGmCmCmUmGmCmGmGmGmCmC | | | | |
| | | | mGmGmCmCmUmCmAmGmCmUmUdTdC | | | | |
| | | | dCmAmCmUmUmCmCmUmG*mC*mC* | | | | |
| | | | mG*mG-3′ | | | | |
| 72 | KB- | 50 | 5′- | 17.5 | 0.7 | 0.0 | 0.0 |
| | 021- | | mU*mU*mU*mC*mUmUmGmGmGmAmA | | | | |
| | 255 | | mUmGmGmCmGmUmGmAmAmGmGmUmC | | | | |
| | | | mGmGmCmCmUmCmAmGmCmUmUdTdC | | | | |
| | | | dCmAmCmUmUmCmCmUmG*mC*mC* | | | | |
| | | | mG*mG-3′ | | | | |
| 73 | KB- | 50 | 5′- | 20.1 | 1.4 | 0.0 | 0.0 |
| | 021- | | mU*mU*mU*mC*mUmUmGmGmGmAmA | | | | |
| | 256 | | mUmGmGmCmGmUmGmAmAmGmGmUmC | | | | |
| | | | mGmGmCmCmUmCmAmGmCmUmUdGdC | | | | |
| | | | dCmAmCmUmUmCmCmUmG*mC*mC* | | | | |
| | | | mG*mG-3′ | | | | |
| 74 | KB- | 50 | 5′- | 24.6828143* | 0.0 | 11.9 | 4.5 |
| | 021- | | mU*mU*mU*mCUmUmUmGmGmGmAmA | | | | |
| | 257 | | mUmGmGmGmCmUmGmCmGmGmGmUmC | | | | |
| | | | mGmGmCmCmUmCmAmGmCmUmUdTdC | | | | |
| | | | dCmAmCmUmUmCmCmUmG*mC*mC* | | | | |
| | | | mG*mG-3′ | | | | |
| 75 | KB- | 50 | 5′- | 25.93383138* | 0.0 | 0.0 | 0.0 |
| | 021- | | mU*mU*mU*mC*mUmUmGmGmGmAmA | | | | |
| | 258 | | mUmAmGmCmCmCmGmAmGmGmGmUmC | | | | |
| | | | mGmGmCmCmUmCmAmGmCmUmUdTdC | | | | |
| | | | dCmAmCmUmUmCmCmUmG*mC*mC* | | | | |
| | | | mG*mG-3′ | | | | |
| 76 | KB- | 50 | 5′- | 9.8 | 13.9 | 6.2 | 8.8 |
| | 021- | | mU*mU*mU*mC*mUmUmGmGmGmAmA | | | | |
| | 259 | | mUmGmGmCmCmUmGmAmGmGmGmUmC | | | | |
| | | | mGmGmCmCmUmCmAmGmCmUmU [symbol] dC | | | | |
| | | | dCmAmCmUmUmCmCmUmG*mC*mC* | | | | |
| | | | mG*mG-3′ | | | | |
| 77 | KB- | 50 | 5′- | 27.7 | 1.5 | 7.6 | 13.1 |
| | 021- | | mU*mU*mU*mC*mUmUmGmGmGmAmA | | | | |
| | 260 | | mUmGmGmCmCmUmGmAmGmGmGmUmC | | | | |
| | | | mGmGmCmCmUmCmAmGmCmUmUdT [symbol] | | | | |
| | | | dCmAmCmUmUmCmCmUmG*mC*mC* | | | | |
| | | | mG*mG-3′ | | | | |
| 78 | KB- | 50 | 5′- | 0.0 | 0.0 | 26.9406393* | 0.0 |
| | 021- | | mU*mU*mU*mC*mUmUmGmGmGmAmA | | | | |
| | 261 | | mUmGmGmCmCmUmGmAmGmGmGmUmC | | | | |
| | | | mGmGmCmCmUmCmAmGmCmUmUdTdC | | | | |
| | | | [symbol] mAmCmUmUmCmCmUmG*mC*mC* | | | | |
| | | | mG*mG-3 | | | | |
| 79 | KB- | 50 | 5′- | 0.0 | 0.0 | 0.0 | 0.0 |
| | 021- | | mU*mU*mU*mC*mUmUmGmGmGmAmA | | | | |
| | 262 | | mUmGmGmCmCmUmGmAmGmGmGmUmC | | | | |
| | | | GmGmCmCmUmCmAmGmCmUmU [symbol] | | | | |
| | | | [symbol] mAmCmUmUmCmCmUmG*mC*mC* | | | | |
| | | | mG*mG-3′ | | | | |
| 80 | KB- | 50 | 5′- | 26.3 | 6.0 | 31.5 | 6.2 |
| | 021- | | mU*mU*mU*mC*mUmUmGmGmGmAmA | | | | |
| | 263 | | mUmGmGmCmCmUmGmAmGmGmGmUmC | | | | |
| | | | mGmGmCmCmUmCmAmGmCmUmUdT [symbol] | | | | |
| | | | [symbol] mAmCmUmUmCmCmUmG*mC*mC* | | | | |
| | | | mG*mG-3′ | | | | |
| 81 | KB- | 50 | 5′- | 26.3 | 14.7 | 0.0 | 0.0 |
| | 021- | | mU*mU*mU*mC*mUmUmGmGmGmAmA | | | | |
| | 264 | | mUmGmGmCmCmUmGmAmGmGmGmUmC | | | | |
| | | | mGmGmCmCmUmCmAmGmCmUmU [symbol] | | | | |
| | | | dCmAmCmUmUmCmCmUmG*mC*mC* | | | | |
| | | | mG*mG-3′ | | | | |
| 82 | KB- | 50 | 5′- | 0.0 | 0.0 | 0.0 | 0.0 |
| | 021- | | mU*mU*mU*mC*mUmUmGmGmGmAmA | | | | |
| | 589 | | mUmGmGmCmCmUmGmAmGmGmGmUmC | | | | |
| | | | mGmGmCmCmUmCmAmGmCmUmU [symbol] dC | | | | |
| | | | [symbol] mAmCmUmUmCmCmUmG*mC*mC* | | | | |
| | | | mG*mG-3′ | | | | |
| | | | Human MeCP2 R255X- | | | | |
| | | | Antisense-gRNA | | | | |
| | | | (R255X → R255W) | | | | |
| 87 | | 50 | 5′- | | | | |
| | | | mG*mC*mG*mC*mUmUmCmUmUmGmA | | | | |
| | | | mUmGmGmCmGmAmGmUmAmCmGmGmU | | | | |
| | | | mCmUmCmCmUmGmCmAmCmAmGdAdC | | | | |
| | | | dCmAmGmAmUmAmGmAmA*mG*mA* | | | | |
| | | | mC*mU-3′ | | | | |
| 101 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] dC | | | | |
| | | | dCmAmGmAmUmAmGmAmA*mG*mA* | | | | |
| | | | mC*mU-3′ | | | | |
| 102 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdA [symbol] | | | | |
| | | | dCmAmGmAmUmAmGmAmA*mG*mA* | | | | |
| | | | mC*mU-3′ | | | | |
| 103 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdAdC [symbol] | | | | |
| | | | [symbol] mAmGmAmUmAmGmAmA*mG*mA* | | | | |
| | | | mC*mU-3′ | | | | |
| 104 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdA [symbol] | | | | |
| | | | [symbol] mAmGmAmUmAmGmAmA*mG*mA* | | | | |
| | | | mC*mU-3′ | | | | |
| 105 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] | | | | |
| | | | dCmAmGmAmUmAmGmAmA*mG*mA* | | | | |
| | | | mC*mU-3′ | | | | |
| 106 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] | | | | |
| | | | [symbol] mAmGmAmUmAmGmAmA*mG*mA* | | | | |
| | | | mC*mU-3′ | | | | |
| 107 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] dC | | | | |
| | | | dCmAmGmAmUmAmGmAmA*mG*mA* | | | | |
| | | | mC*mU-3′ | | | | |
| 108 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdA [symbol] | | | | |
| | | | dCmAmGmAmUmAmGmAmA*mG*mA* | | | | |
| | | | mC*mU-3′ | | | | |
| 109 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdAdC | | | | |
| | | | [symbol] mAmGmAmUmAmGmAmA*mG*mA* | | | | |
| | | | mC*mU-3′ | | | | |
| 110 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] | | | | |
| | | | CmAmGmAmUmAmGmAmA*mG*mA* | | | | |
| | | | mC*mU-3′ | | | | |
| 111 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmCamA | | | | |
| | | | dCdCmAmGmAmUmAmGmAmA*mG*mA* | | | | |
| | | | mC*mU-3′ | | | | |
| 112 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdAa | | | | |
| | | | mCdCmAmGmAmUmAmGmAmA*mG*mA* | | | | |
| | | | mC*mU-3′ | | | | |
| 113 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdAdC | | | | |
| | | | amCmAmGmAmUmAmGmAmA*mG*mA* | | | | |
| | | | mC*mU-3′ | | | | |
| 114 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdA [symbol] | | | | |
| | | | dCmAmGmAmUmAmGmAmA*mG*mA* | | | | |
| | | | mC*mU-3′ | | | | |
| 115 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdA [symbol] | | | | |
| | | | dCmAmGmAmUmAmGmAmA*mG*mA* | | | | |
| | | | mC*mU-3′ | | | | |
| 116 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdA [symbol] | | | | |
| | | | dCmAmGmAmlmAmGmAmA*mG*mA* | | | | |
| | | | mC*mu-3′ | | | | |
| 117 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdA [symbol] | | | | |
| | | | dCmAmGmAmUmAmGmAmA*mG*mA* | | | | |
| | | | mC*mU-3′ | | | | |
| 118 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] dC | | | | |
| | | | dCmAmGmAmUmAmGmAmA*mG*mA* | | | | |
| | | | mC*mU-3′ | | | | |
| 119 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdA [symbol] | | | | |
| | | | dCmAmGmAmUmAmGmAmA*mG*mA* | | | | |
| | | | mC*mU-3′ | | | | |
| 120 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] | | | | |
| | | | CdCmAmGmAmUmAmGmAmA*mG*mA* | | | | |
| | | | mC*mU-3′ | | | | |
| 121 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGrgAdC | | | | |
| | | | dCmAmGmAmUmAmGmAmA*mG*mA* | | | | |
| | | | mC*mU-3′ | | | | |
| 122 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmCdArgC | | | | |
| | | | dCmAmGmAmUmAmGGmAmA*mG*mA* | | | | |
| | | | mC*mU-3′ | | | | |
| 123 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdAdC | | | | |
| | | | rgCmAmGmAmUmAmGmAmA*mG*mA* | | | | |
| | | | mC*mU-3′ | | | | |
| 124 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] dC | | | | |
| | | | dCmAmGmAmUmAmGmAmA*mG*mA* | | | | |
| | | | mC*mU-3′ | | | | |
| 125 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdA [symbol] | | | | |
| | | | dCmAmGmAmUmAmGmAmA*mG*mA* | | | | |
| | | | mC*mu-3′ | | | | |
| 126 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdAdC [symbol] | | | | |
| | | | CmAmGmAmUmAmGmAmA*mG*mA* | | | | |
| | | | mC*mU-3′ | | | | |
| 127 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] dC | | | | |
| | | | dCmAmGmAmUmAmGmAmA*mG*mA* | | | | |
| | | | mC*mU-3′ | | | | |
| 128 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdA [symbol] | | | | |
| | | | dCmAmGmAmUmAmGmAmA*mG*mA* | | | | |
| | | | mC*mU-3′ | | | | |
| 129 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdAdC [symbol] | | | | |
| | | | [symbol] mAmGmAmUmAmGmAmA*mG*mA* | | | | |
| | | | mC*mU-3 | | | | |
| 130 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] dC | | | | |
| | | | dCmAmGmAmUmAmGmAmA*mG*mA* | | | | |
| | | | mC*mU-3′ | | | | |
| 131 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdA [symbol] | | | | |
| | | | dCmAmGmAmUmAmGmAmA*mG*mA* | | | | |
| | | | mC*mU-3′ | | | | |
| 132 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdAdC | | | | |
| | | | [symbol] mAmCmAmUmAmGmAmA*mG*mA* | | | | |
| | | | mC*mU-3′ | | | | |
| 133 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] dC | | | | |
| | | | dCmAmGmAmUmAmGmAmA*mG*mA* | | | | |
| | | | mC*mU-3′ | | | | |
| 134 | | 50 | 5′- | | | | |
| | | | mG*mC*mG*mC*mUmUmCmUmUmGmA | | | | |
| | | | mUmGmGmCmGmAmGmUmAmCmGmGmU | | | | |
| | | | mCmUmCmCmUmGmCmAmCmAmGdA [symbol] | | | | |
| | | | dCmAmGmAmUmAmGmAmA*mG*mA* | | | | |
| | | | mC*mU-3′ | | | | |
| 135 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdAdC | | | | |
| | | | [symbol] mAmGmAmUmAmGmAmA*mG*mA* | | | | |
| | | | mC*mU-3′ | | | | |
| 136 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] | | | | |
| | | | dCmAmGmAmUmAmCmAmA*mG*mA* | | | | |
| | | | mC*mU-3′ | | | | |
| 137 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdA [symbol] | | | | |
| | | | [symbol] mAmGmAmUmAmGmAmA*mG*mA* | | | | |
| | | | mC*mU-3′ | | | | |
| 138 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmG [symbol] dC | | | | |
| | | | dImAmGmAmUmAmGmAmA*mG*mA* | | | | |
| | | | mC*mU-3′ | | | | |
| 139 | | 50 | 5′- | | | | |
| | | | mG*mC*mG*mC*mUmUmCmUmUmGmA | | | | |
| | | | mUmGmGmGmGmAmGmUmAmCmGmGmU | | | | |
| | | | mCmUmCmCmUmGmCmAmCmAmGdIdI | | | | |
| | | | dImAmGmAmUmAmGmAmA*mG*mA* | | | | |
| | | | mC*mU-3′ | | | | |
| | | | Human MeCP2 R255X- | | | | |
| | | | GluR-gRNA | | | | |
| | | | (R255X → R255W) | | | | |
| 83 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAdCdCmAmGmAmU | | | | |
| | | | mA*mG*mA*mA*mG-3′ | | | | |
| 140 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmG [symbol] dCdCmAmGmAmU | | | | |
| | | | mA*mG*mA*mA*mG-3′ | | | | |
| 141 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdA [symbol] dCmAmGmAmU | | | | |
| | | | mA*mG*mA*mA*mG-3′ | | | | |
| 142 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAdC [symbol] mAmGmAmU | | | | |
| | | | mA*mG*mA*mA*mG-3′ | | | | |
| 143 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmGmCmAmC- | | | | |
| | | | mGmCmAmCmAmGdA [symbol] mAmGmAmU | | | | |
| | | | mA*mG*mA*mA*mG-3′ | | | | |
| 144 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmG [symbol] dCmAmGmAmU | | | | |
| | | | mA*mG*mA*mA*mG-3′ | | | | |
| 145 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmG [symbol] mAmGmAmU | | | | |
| | | | mA*mG*mA*mA*mG-3′ | | | | |
| 146 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmG [symbol] dCdCmAmGmAmU | | | | |
| | | | mA*mG*mA*mA*mG-3′ | | | | |
| 147 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmGmCmAmC- | | | | |
| | | | mGmCmAmCmAmGdA [symbol] dCmAmGmAmU | | | | |
| | | | mA*mG*mA*mA*mG-3′ | | | | |
| 148 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAdCaCmAmGmAmU | | | | |
| | | | mA*mG*mA*mA*mG-3′ | | | | |
| 149 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGaAaCaCmAmGmAmU | | | | |
| | | | mA*mG*mA*mA*mG-3′ | | | | |
| 150 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGamAdCdCmAmGmA | | | | |
| | | | mUmA*mG*mA*mA*mG-3′ | | | | |
| 151 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAamCdCmAmGmA | | | | |
| | | | mUmA*mG*mA*mA*mG-3′ | | | | |
| 152 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAdCamCmAmGmA | | | | |
| | | | mUmA*mG*mA*mA*mG-3′ | | | | |
| 153 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdA [symbol] dCmAmGmAmU | | | | |
| | | | mA*mG*mA*mA*mG-3′ | | | | |
| 154 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdA [symbol] dCmAmGmAmU | | | | |
| | | | mA*mG*mA*mA*mG-3′ | | | | |
| 155 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdA [symbol] dCmAmGmAmU | | | | |
| | | | mA*mG*mA*mA*mG-3′ | | | | |
| 156 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdA [symbol] dCmAmGmAmU | | | | |
| | | | mA*mG*mA*mA*mG-3′ | | | | |
| 157 | | 63 | 5′- | | | | |
| | | (45 + 18) | mG*mU*mG*mG*mAmAmUmAmGmUmA | | | | |
| | | | mUmAmAnCmAmAmUmAmUmGmCmUmA | | | | |
| | | | mAmAmUmGmUmUmGmUmUmAmUmAmG | | | | |
| | | | mUmAmUmCmCmCmAmG- | | | | |
| | | | mGmCmAmCmAmGsgAdCdCmAmGmA | | | | |
| | | | mUmA*mG*mA*mA*mG-3′ | | | | |
| 158 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAsgCdCmAmGmA | | | | |
| | | | mUmA*mG*mA*mA*mG-3′ | | | | |
| 159 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGsgAsgCdCmAmGmA | | | | |
| | | | mUmA*mG*mA*mA*mG-3′ | | | | |
| 160 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGrgAdCdCmAmGmA | | | | |
| | | | mUmA*mG*mA*mA*mG-3′ | | | | |
| 161 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdArgCdCmAmGmA | | | | |
| | | | mUmA*mG*mA*mA*mG-3′ | | | | |
| 162 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAdCrgCmAmGmA | | | | |
| | | | mUmA*mG*mA*mA*mG-3′ | | | | |
| 163 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmG [symbol] dCdCmAmGmAmU | | | | |
| | | | mA*mG*mA*mA*mG-3′ | | | | |
| 164 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdA [symbol] dCmAmGmAmU | | | | |
| | | | mA*mG*mA*mA*mG-3′ | | | | |
| 165 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAdC [symbol] mAmGmAmU | | | | |
| | | | mA*mG*mA*mA*mG-3′ | | | | |
| 166 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmG [symbol] dCdCmAmGmAmU | | | | |
| | | | mA*mG*mA*mA*mG-3′ | | | | |
| 167 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdA [symbol] dCmAmGmAmU | | | | |
| | | | mA*mG*mA*mA*mG-3′ | | | | |
| 168 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAdC [symbol] mAmGmAmU | | | | |
| | | | mA*mG*mA*mA*mG-3′ | | | | |
| 169 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmG [symbol] dCdCmAmGmAmU | | | | |
| | | | mA*mG*mA*mA*mG-3′ | | | | |
| 170 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdA [symbol] dCmAmGmAmU | | | | |
| | | | mA*mG*mA*mA*mG-3′ | | | | |
| 171 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAdC [symbol] mAmGmAmU | | | | |
| | | | mA*mG*mA*mA*mG-3′ | | | | |
| 172 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdIdCdCmAmGmAmU | | | | |
| | | | mA*mG*mA*mA*mG-3′ | | | | |
| 173 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAdIdCmAmGmAmU | | | | |
| | | | mA*mG*mA*mA*mG-3′ | | | | |
| 174 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAdCdImAmGmAmU | | | | |
| | | | mA*mG*mA*mA*mG-3′ | | | | |
| 175 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdIdIdCmAmGmAmU | | | | |
| | | | mA*mG*mA*mA*mG-3′ | | | | |
| 176 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdAdIdImAmGmAmU | | | | |
| | | | mA*mG*mA*mA*mG-3′ | | | | |
| 177 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdIdCdImAmGmAmU | | | | |
| | | | mA*mG*mA*mA*mG-3 | | | | |
| 178 | | 63 | 5′- | | | | |
| | | (45 + 18) | mG*mU*mG*mG*mAmAmUmAmGmUmA | | | | |
| | | | mUmAmAmCmAmAmUmAmUmGmCmUmA | | | | |
| | | | mAmAmUmGmUmUmGmUmUmAmUmAmG | | | | |
| | | | mUmAmUmCmCmCmAmC- | | | | |
| | | | mGmCmAmCmAmGdIdIdImAmGmAmU | | | | |
| | | | mA*mG*mA*mA*mG-3′ | | | | |

[symbol]

indicates data missing or illegible when filed

Mismatches outside of the triplet were generally well tolerated and allowed to improve the editing activity. Relative to the editing observed for a guide oligonucleotide that is fully 2′-OMe modified with a DNA triplet, generally editing was observed to be higher for guide oligonucleotides comprising a FANA triplet.

### Example 2. Reversing an Amino Acid Substitution Mutation R294X in the MECP2 Transcript by Targeted a to I Editing

Guide oligonucleotides were chemically synthesized as previously described in Example 1.

Human ADAR2 sequence (NM_001112.4; SEQ ID NO: 55), human ADAR1p110 (NM_001111.5; SEQ ID NO: 56) and human MECP2 R294X (NM_004992; SEQ ID NO: 59) sequences (ORF only), were cloned into pcDNA3.1 plasmid under the control of the CMV promoter using BamHI and XbaI restriction sites (Quintara Bio, Berkeley, Calif.) and the correct insert was sequence verified. For the human MECP2 R294X construct, recombinant HA-tag is placed in-frame at the N-terminus of the coding sequence. The plasmids will henceforth be denoted as ADAR2/pcDNA3.1, ADAR1p110/pcDNA3.1, or MECP2/pcDNA3.1. Editing experiments were performed as previously described in Example 1.

TABLE 6
Primers Used for RT-PCR

| | | SEQ |
|---|---|---|
| Name | Sequence (5′ to 3′) | ID NO. |
| MECP2 | AGAAAAGTCCTGGGAAGCTC | 99 |
| R294X Forward | | |
| MECP2 | GGACTCTGAGTGGTGGTGAT | 100 |
| R294X Reverse | | |

ADAR mediated editing of exemplary modified guide oligonucleotides targeting human MECP2 R294X are described in Table 5.

Abbreviations of various modifications are shown in the table below. For example, A, C, G and U are ribonucleosides; dA, dC, dG and dT are deoxyribonucleosides; mA, mC, mG and mU are 2′-O-methyl ribonucleosides; fA, fC, fG and fU are 2′-fluoroarabinonucleosides (FANA). The DNA triplet (dTdCdG) and the FANA triplet (fUfCfG) are indicated in bold. Mismatches are indicated in bold and underline. GluR refers to the natural ADAR substrate, GluR2 receptor pre-mRNA or longer RNA sequences that form a hairpin structure similar to GluR2.

| Modification | Abbreviation |
|---|---|
| RNA | rN |
| DNA | dN |
| 2′-O-Methyl(2′-OMe) | mN |
| 2′-F-arabinose (FANA) | fN |
| 2′-F-RNA | FN |
| 2′-O-Methoxyethyl (MOE) | MN |
| Locked Nucleic Acids (LNA) | LN |
| Abasic | dS |
| RNA-Abasic | rs |
| 2′-OMe-Abasic | mS |
| 5-Methyl-rC | rmC |
| 5-Methyl-dC | dmC |
| 5-Methyl-2′-OMe-C | mmC |
| (S)-Glycol Nucleic Acid (S-GNA) | sgN |
| (R)-Glycol Nucleic Acid (R-GNA) | rgN |
| DNA α-anomer | αN |
| Arabinose | aN |
| 2′-OMe-Arabinose | amN |
| Flexible Nucleic Acid (FNA) | fxN |
| Serineol Nucleic Acid | sN |
| β-D-homoDNA | hdN |
| β-D-HNA | hN |
| Phosphorothioate internucleoside linkage | * |

TABLE 7
Guide Oligonucleotides Targeting Human MECP2 (R294X)

| | | | | | | ADAR1 | ADAR1 |
|---|---|---|---|---|---|---|---|
| SEQ | | | | ADAR | ADAR2 | p1110 | p110 |
| ID | Oligo | | | 2% | St. | % | St. |
| NO. | ID | Length | Guide RNA Sequences | editing | Dev. | Editing | Dev. |
| | | | Human MeCP2 R294X- | | | | |
| | | | GluR-gRNA | | | | |
| | | | (R294X → [symbol] ) | | | | |
| 83 | KB- | 63 | 5′- | 0.0 | 0.0 | 0 | 0 |
| | 021- | | mG*mU*mG*mG*mAmAmUmAmGmUmAmU | | | | |
| | 265 | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mGmCmAmCmAmGdAdCdCmAmGmAmU | | | | |
| | | | mA*mG*mA*mA*mG-3′ | | | | |
| 84 | KB- | 65 | 5′- | 14.8663102* | 0.0 | 0 | 0 |
| | 021- | | mG*mU*mG*mG*mAmAmGmAmGmUmAmU | | | | |
| | 266 | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmCmAmCmAmGdAdCdCmAmGmAmUmA | | | | |
| | | | mGmA*mA*mG*mA*mC-3′ | | | | |
| 85 | KB- | 63 | 5′- | 0.0 | 0.0 | 0 | 0 |
| | 021- | | mG*mG*mU*mG*mAmAmGmAmGmGmAmG | | | | |
| | 267 | | mAmAmCmAmAmUmAmUmGmCmUmAmAmA | | | | |
| | | | mUmGmUmUmGmUmUmCmUmCmGmUmCmU | | | | |
| | | | mCmCmAmCmC- | | | | |
| | | | mGmCmAmCmAmGdAdCdCmAmGmAmU | | | | |
| | | | mA*mG*mA*mA*mG-3′ | | | | |
| 86 | KB- | 73 | 5′- | 0.0 | 0.0 | 0 | 0 |
| | 021- | | mG*mG*mU*mG*mUmCmGmAmGmAmAmG | | | | |
| | 268 | | mAmGmGmAmGmAmAmCmAmAmUmAmUmG | | | | |
| | | | mCmUmAmAmAmUmGmUmUmGmUmUmCmU | | | | |
| | | | mCmGmUmCmUmCmCmUmCmGmAmCmAmC | | | | |
| | | | mC- | | | | |
| | | | mGmCmAmCmAmGdAdCdCmAmGmAmU | | | | |
| | | | mA*mG*mA*mA*mG-3′ | | | | |
| | | | Human MeCP2 R294X- | | | | |
| | | | Antisense-gRNA | | | | |
| | | | (R294X → [symbol] ) | | | | |
| 87 | KB- | 50 | 5′- | 0.0 | 0.0 | 13.4 | 0.8 |
| | 021- | | mG*mC*mG*mC*mUmUmCmUmUmGmAmU | | | | |
| | 269 | | mGmGmGmGmAmGmUmAmCmGmGmUmCmU | | | | |
| | | | mCmCmUmGmCmAmCmAmGdAdCdCmAmG | | | | |
| | | | mAmUmAmGmAmA*mG*mA*mC*mU-3′ | | | | |
| 88 | KB- | 50 | 5′- | 0.0 | 0.0 | 0.0 | 0.0 |
| | 021- | | mG*mC*mG*mC*mUmUmCmUmUmGmAmU | | | | |
| | 270 | | mGmGmGmGmAmGmUmAmGmGmGmUmGmU | | | | |
| | | | mCmCmUmGmCmAmCmAmGdAdCdCmAmG | | | | |
| | | | mAmUmAmGmAmA*mG*mA*mC*mU-3′ | | | | |
| 89 | KB- | 50 | 5′- | 0.0 | 0.0 | 12.6626193* | 0.0 |
| | 021- | | mG*mC*mG*mC*mUmUmCmUmUmGmAmU | | | | |
| | 271 | | mGmGmGmGmAmGmUmCmCmGmGmCmCmU | | | | |
| | | | mCmCmUmGmCmAmCmAmGdAdCdCmAmG | | | | |
| | | | mAmUmAmGmAmA*mG*mA*mC*mU-3′ | | | | |
| 90 | KB- | 50 | 5′- | 0.0 | 0.0 | 0.0 | 0.0 |
| | 021- | | mG*mC*mG*mC*mUmUmCmUmUmGmAmU | | | | |
| | 272 | | mGmGmGmGmAmAmUmAmCmAmGmUmCmU | | | | |
| | | | mCmCmUmGmCmAmCmAmGdAdCdCmAmG | | | | |
| | | | mAmUmAmGmAmA*mG*mA*mC*mU-3′ | | | | |
| 91 | KB- | 50 | 5′- | 11.6144019* | 0.0 | 9.3 | 1.4 |
| | 021- | | mG*mC*mG*mC*mUmUmCmUmUmGmAmU | | | | |
| | 273 | | mGmGmGmAmAmGmUmCmCmGmGmUmCmU | | | | |
| | | | mCmCmUmGmCmAmCmAmGdAdCdCmAmG | | | | |
| | | | mAmUmAmGmAmA*mG*mA*mC*mU-3′ | | | | |
| 92 | KB- | 50 | 5′ | 0.0 | 0.0 | 10.4029991* | 0.0 |
| | 021- | | mG*mC*mG*mC*mUmUmCmUmUmGmAmU | | | | |
| | 274 | | mGmGmAmGmAmGmCmAmCmGmGmUmCmU | | | | |
| | | | mCmCmUmGmCmAmCmAmGdAdCdCmAmG | | | | |
| | | | mAmUmAmGmAmA*mG*mA*mC*mU-3′ | | | | |
| 93 | KB- | 50 | 5′- | 13.6 | 11.9 | 15.4 | 1.7 |
| | 021- | | mG*mC*mG*mC*mUmUmCmUmUmGmAmU | | | | |
| | 275 | | mGmGmGmAmAmGmUmCmCmGmGmUmCmU | | | | |
| | | | mCmCmUmGmCmAmCmAmG [symbol] dCdCmAmG | | | | |
| | | | mAmUmAmGmAmA*mG*mA*mC*mU-3′ | | | | |
| 94 | KB- | 50 | 5′- | 21.4 | 3.3 | 16.2 | 10.9 |
| | 021- | | mG*mC*mG*mC*mUmUmCmUmUmGmAmU | | | | |
| | 276 | | mGmGmGmAmAmGmUmCmCmGmGmUmCmU | | | | |
| | | | mCmCmUmGmCmAmCmAmGdA [symbol] dCmAmG | | | | |
| | | | mAmUmAmGmAmA*mG*mA*mC*mU-3′ | | | | |
| 95 | KB- | 50 | 5′- | 17.4 | 3.5 | 0.0 | 0.0 |
| | 021- | | mG*mC*mG*mC*mUmUmCmUmUmGmAmU | | | | |
| | 277 | | mGmGmGmAmAmGmUmCmCmGmGmUmCmU | | | | |
| | | | mCmCmUmGmCmAmCmAmGdAdC [symbol] mAmG | | | | |
| | | | mAmUmAmGmAmA*mG*mA*mC*mU-3′ | | | | |
| 96 | KB- | 50 | 5′- | 18.1 | 3.4 | 9.7 | 8.6 |
| | 021- | | mG*mC*mG*mC*mUmUmCmUmUmGmAmU | | | | |
| | 278 | | mGmGmGmAmAmGmUmCmCmGmGmUmCmU | | | | |
| | | | mCmCmUmGmCmAmCmAmG [symbol] mAmG | | | | |
| | | | mAmUmAmGmAmA*mG*mA*mC*mU-3′ | | | | |
| 97 | KB- | 50 | 5′- | 11.2 | 1.5 | 21.8 | 5.7 |
| | 021- | | mG*mC*mG*mC*mUmUmCmUmUmGmAmU | | | | |
| | 279 | | mGmGmGmAmAmGmUmCmCmGmGmUmCmU | | | | |
| | | | mCmCmUmGmCmAmCmAmGdA [symbol] mAmG | | | | |
| | | | mAmUmAmGmAmA*mG*mA*mC*mU-3′ | | | | |
| 98 | KB- | 50 | 5′- | 9.4 | 13.2 | 18.2237601* | 0.0 |
| | 021- | | mG*mC*mG*mC*mUmUmCmUmUmGmAmU | | | | |
| | 280 | | mGmGmGmAmAmGmUmCmCmGmGmUmCmU | | | | |
| | | | mCmCmUmGmCmAmCmAmG [symbol] dCmAmG | | | | |
| | | | mAmUmAmGmAmA*mG*mA*mC*mU-3′ | | | | |
| | | | Human MeCP2 R294X- | | | | |
| | | | Antisense-gRNA | | | | |
| | | | (R294X → [symbol] ) | | | | |
| 66 | | 50 | 5′- | | | | |
| | | | mU*mU*mU*mC*mUmUmGmCmGmAmAmU | | | | |
| | | | mGmGmCmCmUmGmAmGmGmGmUmCmGmG | | | | |
| | | | mCmCmUmCmAmGmCmUmUdTdCdCmAmC | | | | |
| | | | mUmUmCmCmUmG*mC*mC*mG*mG-3′ | | | | |
| 76 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmU [symbol] dCdCmAmC | | | | |
| | | | mUmUmCmCmUmG*mC*mC*mG*mG-3′ | | | | |
| 77 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdT [symbol] dCmAmC | | | | |
| | | | mUmUmCmCmUmG*mC*mC*mG*mG-3′ | | | | |
| 78 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTdC [symbol] mAmC | | | | |
| | | | mUmUmCmCmUmG*mC*mC*mG*mG-3′ | | | | |
| 80 | | 50 | 5′- | | | | |
| | | | mU*mU*mU*mC*mUmUmGmGmGmAmAmU | | | | |
| | | | mGmGmCmCmUmGmAmCmGmGmUmCmGmG | | | | |
| | | | mCmCmUmCmAmGmCmUmUdT [symbol] mAmC | | | | |
| | | | mUmUmCmCmUmG*mC*mC*mG*mG-3′ | | | | |
| 81 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmU [symbol] dCmAmC | | | | |
| | | | mUmUmCmCmUmG*mC*mC*mG*mG-3′ | | | | |
| 79 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUm [symbol] mAmC | | | | |
| | | | mUmUmCmCmUmG*mC*mC*mG*mG-3′ | | | | |
| 179 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmU [symbol] dCdCmAmC | | | | |
| | | | mUmUmCmCmUmG*mC*mC*mG*mG-3′ | | | | |
| 180 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdT [symbol] dCmAmC | | | | |
| | | | mUmUmCmCmUmG*mC*mC*mG*mG-3′ | | | | |
| 181 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTdCaCmAmC | | | | |
| | | | mUmUmCmCmUmG*mC*mC*mG*mG-3′ | | | | |
| 182 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUaUaCaCmAmC | | | | |
| | | | mUmUmCmCmUmG*mC*mC*mG*mG-3′ | | | | |
| 183 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUamUdCdCmAmC | | | | |
| | | | mUmUmCmCmUmG*mC*mC*mG*mG-3′ | | | | |
| 184 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTamCdCmAmC | | | | |
| | | | mUmUmCmCmUmG*mC*mC*mG*mG-3′ | | | | |
| 185 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTdCamCmAmC | | | | |
| | | | mUmUmCmCmUmG*mC*mC*mG*mG-3′ | | | | |
| 186 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdT [symbol] dCmAmC | | | | |
| | | | mUmUmCmCmUmG*mC*mC*mG*mG-3′ | | | | |
| 187 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTdSdCmAmC | | | | |
| | | | mUmUmCmCmUmG*mC*mC*mG*mG-3′ | | | | |
| 188 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTrSdCmAmC | | | | |
| | | | mUmUmCmCmUmG*mC*mC*mG*mG-3′ | | | | |
| 189 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdT [symbol] dCmAmC | | | | |
| | | | mUmUmCmCmUmG*mC*mC*mG*mG-3′ | | | | |
| 190 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUsgTdCdCmAmC | | | | |
| | | | mUmUmCmCmUmG*mC*mC*mG*mG-3′ | | | | |
| 191 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTsgCdCmAmC | | | | |
| | | | mUmUmCmCmUmG*mC*mC*mG*mG-3′ | | | | |
| 192 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUsgTsgCdCmA | | | | |
| | | | mCmUmUmCmCmUmG*mC*mC*mG*mG-3′ | | | | |
| 193 | | 50 | 5′- | | | | |
| | | | mU*mU*mU*mC*mUmUmCmGmGmAmAmU | | | | |
| | | | mGmGmCmCmUmGmAmGmGmGmUmCmGmG | | | | |
| | | | mCmCmUmCmAmGmCmUmUrgTdCdCmAmC | | | | |
| | | | mUmUmCmCmUmG*mC*mC*mG*mG-3′ | | | | |
| 194 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTrgCdCmAmC | | | | |
| | | | mUmUmCmCmUmG*mC*mC*mG*mG-3′ | | | | |
| 195 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTdCrgCmAmC | | | | |
| | | | mUmUmCmCmUmG*mC*mC*mG*mG-3′ | | | | |
| 196 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmU [symbol] dCdCmAmC | | | | |
| | | | mUmUmCmCmUmG*mC*mC*mG*mG-3′ | | | | |
| 197 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdT [symbol] dCmAmC | | | | |
| | | | mUmUmCmCmUmG*mC*mC*mG*mG-3′ | | | | |
| 198 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTdC [symbol] mAmCm | | | | |
| | | | UmUmCmCmUmG*mC*mC*mG*mG-3′ | | | | |
| 199 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmU [symbol] dCdCmAmC | | | | |
| | | | mUmUmCmCmUmG*mC*mC*mG*mG-3′ | | | | |
| 200 | | 50 | 5′- | | | | |
| | | | mUmUmUmC*mUmUmGmGmGmAmAmUm | | | | |
| | | | GmGmCmCmUmGmAmGmGmGmUmCmGmG | | | | |
| | | | mCmCmUmCmAmGmCmUmUdT [symbol] dCmAmC | | | | |
| | | | mUmUmCmCmUmG*mC*mC*mG*mG-3′ | | | | |
| 201 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTdC [symbol] mAmC | | | | |
| | | | mUmUmCmCmUmG*mC*mC*mG*mG-3′ | | | | |
| 202 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmU [symbol] dCdCmAmC | | | | |
| | | | mUmUmCmCmUmG*mC*mC*mG*mG-3′ | | | | |
| 203 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdT [symbol] dCmAmC | | | | |
| | | | mUmUmCmCmUmG*mC*mC*mG*mG-3′ | | | | |
| 204 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTdC [symbol] mAmC | | | | |
| | | | mUmUmCmCmUmG*mC*mC*mG*mG-3′ | | | | |
| 205 | | 50 | 5′- | | | | |
| | | | mU*mU*mU*mC*mUmUmCmGmGmAmAmU | | | | |
| | | | mGmGmCmCmUmGmAmGmGmGmUmCmGmG | | | | |
| | | | mCmCmUmCmAmGmCmUmUdIdCdCmAmC | | | | |
| | | | mUmUmCmCmUmG*mC*mC*mG*mG-3′ | | | | |
| 206 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTdIdCmAmC | | | | |
| | | | mUmUmCmCmUmG*mC*mC*mG*mG-3′ | | | | |
| 207 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTdCdImAmC | | | | |
| | | | mUmUmCmCmUmG*mC*mC*mG*mG-3′ | | | | |
| 208 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdIdIdCmAmC | | | | |
| | | | mUmUmCmCmUmG*mC*mC*mG*mG-3′ | | | | |
| 209 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdTdIdImAmC | | | | |
| | | | mUmUmCmCmUmG*mCmC*mG*mG-3′ | | | | |
| 210 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdIdCdImAmC | | | | |
| | | | mUmUmCmCmUmG*mC*mC*mG*mG-3′ | | | | |
| 211 | | 50 | 5′- | | | | |
| | | | mU*mU*mU*mC*mUmUmGmGmGmAmAmU | | | | |
| | | | mGmGmCmCmUmGmAmGmGmGmUmCmGmG | | | | |
| | | | mCmCmUmCmAmGmCmUmUdIdIdImAmC | | | | |
| | | | mUmUmCmCmUmG*mC*mC*mG*mG-3′ | | | | |
| | | | Human MeCP2 R294X- | | | | |
| | | | GluR-gRNA | | | | |
| | | | (R294X → [symbol] ) | | | | |
| 62 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTdCdCmAmCmUmU | | | | |
| | | | mC*mC*mU*mG*mC-3′ | | | | |
| 212 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmU [symbol] dCdCmAmCmUmU | | | | |
| | | | mC*mC*mU*mG*mC-3′ | | | | |
| 213 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdT [symbol] dCmAmCmUmU | | | | |
| | | | mC*mC*mU*mG*mC-3′ | | | | |
| 214 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTdC [symbol] mAmCmUmU | | | | |
| | | | mC*mC*mU*mG*mC-3′ | | | | |
| 215 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdT [symbol] mAmCmUmU | | | | |
| | | | mC*mC*mU*mG*mC-3′ | | | | |
| 216 | | 63 | 5′- | | | | |
| | | (45 + 18) | mC*mU*mG*mG*mAmAmUmAmCmUnAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmU [symbol] dCmAmCmUmU | | | | |
| | | | mC*mC*mU*mG*mC-3′ | | | | |
| 217 | | 63 | 5′- | | | | |
| | | (45 + 18) | mC*mU*mG*mG*mAmAmUmAmCmUnAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmU [symbol] mAmCmUmU | | | | |
| | | | mC*mC*mU*mG*mC-3′ | | | | |
| 218 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmCmUmUmAmUmAmGmUmAmU | | | | |
| | | | mCmCmCmAmC- | | | | |
| | | | mCmAmGmCmUmU [symbol] dCdCmAmCmUmU | | | | |
| | | | mC*mC*mU*mG*mC-3′ | | | | |
| 219 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdT [symbol] dCmAmCmUmU | | | | |
| | | | mC*mC*mU*mG*mC-3′ | | | | |
| 220 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTdC [symbol] mAmCmUmU | | | | |
| | | | mC*mC*mU*mG*mC-3′ | | | | |
| 221 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmU [symbol] mAmCmUmU | | | | |
| | | | mC*mC*mU*mG*mC-3′ | | | | |
| 222 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUamUdCdCmAmCmUmU | | | | |
| | | | mC*mC*mU*mG*mC-3′ | | | | |
| 223 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTamCdCmAmCmUmU | | | | |
| | | | mC*mC*mU*mG*mC-3′ | | | | |
| 224 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTdCamCmAmCmUmU | | | | |
| | | | mC*mC*mU*mG*mC-3′ | | | | |
| 225 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdT [symbol] dCmAmCmUmU | | | | |
| | | | mC*mC*mU*mG*mC-3′ | | | | |
| 226 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTdSdCmAmCmUmU | | | | |
| | | | mC*mC*mU*mG*mC-3′ | | | | |
| 227 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdT [symbol] dCmAmCmUmU | | | | |
| | | | mC*mC*mU*mG*mC-3′ | | | | |
| 228 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdT [symbol] dCmAmCmUmU | | | | |
| | | | mC*mC*mU*mG*mC-3′ | | | | |
| 229 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUsgTdCdCmAmCmUmU | | | | |
| | | | mC*mC*mU*mG*mC-3′ | | | | |
| 230 | | 63 | 5′- | | | | |
| | | (45 + 18) | mG*mU*mG*mG*mAmAmUmAmGmUmAmU | | | | |
| | | | mAmAmCmAmAmUmAmUmCmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTsgCdCmAmCmUmU | | | | |
| | | | mC*mC*mU*mG*mC-3′ | | | | |
| 231 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUsgTsgCdCmAmCmUmU | | | | |
| | | | mC*mC*mU*mG*mC-3′ | | | | |
| 232 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mGmCmCmAmC- | | | | |
| | | | mCmAmGmCmUmUrgTdCdCmAmCmUmU | | | | |
| | | | mC*mC*mU*mG*mC-3′ | | | | |
| 233 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTrgCdCmAmCmUmU | | | | |
| | | | mC*mC*mU*mG*mC-3′ | | | | |
| 234 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTdCrgCmAmCmUmU | | | | |
| | | | mC*mC*mU*mG*mC-3′ | | | | |
| 235 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmU [symbol] dCdCmAmCmUmU | | | | |
| | | | mC*mC*mU*mG*mC-3′ | | | | |
| 236 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdT [symbol] dCmAmCmUmU | | | | |
| | | | mC*mC*mU*mG*mC-3′ | | | | |
| 237 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTdC [symbol] mAmCmUmU | | | | |
| | | | mC*mC*mU*mG*mC-3′ | | | | |
| 238 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmU [symbol] dCdCmAmCmUmU | | | | |
| | | | mC*mC*mU*mG*mC-3′ | | | | |
| 239 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdT [symbol] dCmAmCmUmU | | | | |
| | | | mC*mC*mU*mG*mC-3′ | | | | |
| 240 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTdC [symbol] mAmCmUmU | | | | |
| | | | mC*mC*mU*mG*mC-3′ | | | | |
| 241 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmU [symbol] dCdCmAmCmUmU | | | | |
| | | | mC*mC*mU*mG*mC-3′ | | | | |
| 242 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdT [symbol] dCmAmCmUmU | | | | |
| | | | mC*mC*mU*mG*mC-3′ | | | | |
| 243 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTdC [symbol] mAmCmUmU | | | | |
| | | | mC*mC*mU*mG*mC-3′ | | | | |
| 244 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdIdCdCmAmCmUmU | | | | |
| | | | mC*mC*mU*mG*mC-3′ | | | | |
| 245 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTdIdCmAmCmUmU | | | | |
| | | | mC*mC*mU*mG*mC-3′ | | | | |
| 246 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTdCdImAmCmUmU | | | | |
| | | | mC*mC*mU*mG*mC-3′ | | | | |
| 247 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdIdIdCmAmCmUmU | | | | |
| | | | mC*mC*mU*mG*mC-3′ | | | | |
| 248 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdTdIdImAmCmUmU | | | | |
| | | | mC*mC*mU*mG*mC-3′ | | | | |
| 249 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdIdCdImAmCmUmU | | | | |
| | | | mC*mC*mU*mG*mC-3′ | | | | |
| 250 | | 63 | 5′- | | | | |
| | | (45 + 18) | mG*mU*mG*mG*mAmAmUmAmGmUmAmU | | | | |
| | | | mAmAmCmAmAmUmAmUmGmCmUmAmAmA | | | | |
| | | | mUmGmUmUmGmUmUmAmUmAmGmUmAmU | | | | |
| | | | mCmCmCmAmC- | | | | |
| | | | mCmAmGmCmUmUdIdIdImAmCmUmU | | | | |
| | | | mC*mC*mU*mG*mC-3′ | | | | |

[symbol]

indicates data missing or illegible when filed

Mismatches outside of the triplet were generally well tolerated and allowed to improve the editing activity. Relative to the editing observed for a guide oligonucleotide that is fully 2′-OMe modified with a DNA triplet, generally editing was observed to be higher for guide oligonucleotides comprising a FANA triplet.

### OTHER EMBODIMENTS

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

INFORMAL SEQUENCE LISTING
SEQ ID NO: 1
GGUGAAUAGUAUAACAAUAU
SEQ ID NO: 2
AUGUUGUUAUAGUAUCCACC
SEQ ID NO: 3
GGUGAAUAGUAUAACAAUAUGCUAAAUGUUGUUAUAGUAUCCACC
SEQ ID NO: 4
GGUGAAGAGGAGAACAAUAU
SEQ ID NO: 5
AUGUUGUUCUCGUCUCCACC
SEQ ID NO: 6
GGUGAAGAGGAGAACAAUAUGCUAAAUGUUGUUCUCGUCUCCACC
SEQ ID NO: 7
GGUGUCGAGAAGAGGAGAACAAUAU
SEQ ID NO: 8
AUGUUGUUCUCGUCUCCUCGACACC
SEQ ID NO: 9
GGUGUCGAGAAGAGGAGAACAAUAUGCUAAAUGUUGUUCUCGUCUCCUCGACACC
SEQ ID NO: 10
GGGUGGAAUAGUAUAACAAUAU
SEQ ID NO: 11
AUGUUGUUAUAGUAUCCCACCU
SEQ ID NO: 12
GGGUGGAAUAGUAUAACAAUAUGCUAAAUGUUGUUAUAGUAUCCCACCU
SEQ ID NO: 13
GUGGAAUAGUAUAACAAUAU
SEQ ID NO: 14
AUGUUGUUAUAGUAUCCCAC
SEQ ID NO: 15
GUGGAAUAGUAUAACAAUAUGCUAAAUGUUGUUAUAGUAUCCCAC
SEQ ID NO: 16
GGUGUCGAGAAUAGUAUAACAAUAU
SEQ ID NO: 17
AUGUUGUUAUAGUAUCCUCGACACC
SEQ ID NO: 18
GGUGUCGAGAAUAGUAUAACAAUAUGCUAAAUGUUGUUAUAGUAUCCUCGACACC
SEQ ID NO: 19
GGGUGGAAUAGUAUAACAAUAU
SEQ ID NO: 20
AUGUUGUUAUAGUAUCCCACCU
SEQ ID NO: 21
GGGUGGAAUAGUAUAACAAUAUGCUAAAUGUUGUUAUAGUAUCCCACCU
SEQ ID NO: 22
GGGUGGAAUAGUAUACCA
SEQ ID NO: 23
UGGUAUAGUAUCCCACCU
SEQ ID NO: 24
GGGUGGAAUAGUAUACCAUUCGUGGUAUAGUAUCCCACCU
SEQ ID NO: 25
GUGGGUGGAAUAGUAUACCA
SEQ ID NO: 26
UGGUAUAGUAUCCCACCUAC
SEQ ID NO: 27
GUGGGUGGAAUAGUAUACCAUUCGUGGUAUAGUAUCCCACCUAC
SEQ ID NO: 28
UGGGUGGAAUAGUAUACCA
SEQ ID NO: 29
UGGUAUAGUAUCCCACCUA
SEQ ID NO: 30
UGGGUGGAAUAGUAUACCAUUCGUGGUAUAGUAUCCCACCUA
SEQ ID NO: 31
GGUGGAAUAGUAUACCA
SEQ ID NO: 32
UGGUAUAGUAUCCCACC
SEQ ID NO: 33
GGUGGAAUAGUAUACCAUUCGUGGUAUAGUAUCCCACC
SEQ ID NO: 34
GUGGAAUAGUAUACCA
SEQ ID NO: 35
UGGUAUAGUAUCCCAC
SEQ ID NO: 36
GUGGAAUAGUAUACCAUUCGUGGUAUAGUAUCCCAC
SEQ ID NO: 37
GGUGAAUAGUAUAACAAUAUGCUAAAUGUUGUUAUAGUAUCCACC
SEQ ID NO: 38
GGUGAAGAGGAGAACAAUAUGCUAAAUGUUGUUCUCGUCUCCACC
SEQ ID NO: 39
GGUGUCGAGAAGAGGAGAACAAUAUGCUAAAUGUUGUUCUCGUCUCCUCGACACC
SEQ ID NO: 40
*s*s*G**GAGAAGAGGAGAA*AA*A*G**AAA*G**G*****G*******GA*A**
SEQ ID NO: 41
GGGUGGAAUAGUAUAACAAUAUGCUAAAUGUUGUUAUAGUAUCCCACCU
SEQ ID NO: 42
GUGGAAUAGUAUAACAAUAUGCUAAAUGUUGUUAUAGUAUCCCAC
SEQ ID NO: 43
GGUGUCGAGAAUAGUAUAACAAUAUGCUAAAUGUUGUUAUAGUAUCCUCGACACC
SEQ ID NO: 44
GGGUGGAAUAGUAUAACAAUAUGCUAAAUGUUGUUAUAGUAUCCCACCU
SEQ ID NO: 45
GGGUGGAAUAGUAUACCAUUCGUGGUAUAGUAUCCCACCU
SEQ ID NO: 46
GUGGGUGGAAUAGUAUACCAUUCGUGGUAUAGUAUCCCACCUAC
SEQ ID NO: 47
UGGGUGGAAUAGUAUACCAUUCGUGGUAUAGUAUCCCACCUA
SEQ ID NO: 48
GGUGGAAUAGUAUACCAUUCGUGGUAUAGUAUCCCACC
SEQ ID NO: 49
GUGGAAUAGUAUACCAUUCGUGGUAUAGUAUCCCAC
SEQ ID NO: 50
ACATGAGGATCACCCATGT
SEQ ID NO: 51
AAVALLPAVLLALLAP
SEQ ID NO: 52
AALLPVLLAAP
SEQ ID NO: 53
GRKKRRQRRRPPQ
SEQ ID NO: 54
RQIKIWFQNRRMKWKK
SEQ ID NO: 55
ATGGAACAAAAACTTATTTCTGAAGAAGATCTGGATATAGAAGATGAAGAAAACATGAGTTCCA
GCAGCACTGATGTGAAGGAAAACCGCAATCTGGACAACGTGTCCCCCAAGGATGGCAGCACACC
TGGGCCTGGCGAGGGCTCTCAGCTCTCCAATGGGGGTGGTGGTGGCCCCGGCAGAAAGCGGCCC
CTGGAGGAGGGCAGCAATGGCCACTCCAAGTACCGCCTGAAGAAAAGGAGGAAAACACCAGGGC
CCGTCCTCCCCAAGAACGCCCTGATGCAGCTGAATGAGATCAAGCCTGGTTTGCAGTACACACT
CCTGTCCCAGACTGGGCCCGTGCACGCGCCTTTGTTTGTCATGTCTGTGGAGGTGAATGGCCAG
GTTTTTGAGGGCTCTGGTCCCACAAAGAAAAAGGCAAAACTCCATGCTGCTGAGAAGGCCTTGA
GGTCTTTCGTTCAGTTTCCTAATGCCTCTGAGGCCCACCTGGCCATGGGGAGGACCCTGTCTGT
CAACACGGACTTCACATCTGACCAGGCCGACTTCCCTGACACGCTCTTCAATGGTTTTGAAACT
CCTGACAAGGCGGAGCCTCCCTTTTACGTGGGCTCCAATGGGGATGACTCCTTCAGTTCCAGCG
GGGACCTCAGCTTGTCTGCTTCCCCGGTGCCTGCCAGCCTAGCCCAGCCTCCTCTCCCTGTCTT
ACCACCATTCCCACCCCCGAGTGGGAAGAATCCCGTGATGATCTTGAACGAACTGCGCCCAGGA
CTCAAGTATGACTTCCTCTCCGAGAGCGGGGAGAGCCATGCCAAGAGCTTCGTCATGTCTGTGG
TCGTGGATGGTCAGTTCTTTGAAGGCTCGGGGAGAAACAAGAAGCTTGCCAAGGCCCGGGCTGC
GCAGTCTGCCCTGGCCGCCATTTTTAACTTGCACTTGGATCAGACGCCATCTCGCCAGCCTATT
CCCAGTGAGGGTCTTCAGCTGCATTTACCGCAGGTTTTAGCTGACGCTGTCTCACGCCTGGTCC
TGGGTAAGTTTGGTGACCTGACCGACAACTTCTCCTCCCCTCACGCTCGCAGAAAAGTGCTGGC
TGGAGTCGTCATGACAACAGGCACAGATGTTAAAGATGCCAAGGTGATAAGTGTTTCTACAGGA
ACAAAATGTATTAATGGTGAATACATGAGTGATCGTGGCCTTGCATTAAATGACTGCCATGCAG
AAATAATATCTCGGAGATCCTTGCTCAGATTTCTTTATACACAACTTGAGCTTTACTTAAATAA
CAAAGATGATCAAAAAAGATCCATCTTTCAGAAATCAGAGCGAGGGGGGTTTAGGCTGAAGGAG
AATGTCCAGTTTCATCTGTACATCAGCACCTCTCCCTGTGGAGATGCCAGAATCTTCTCACCAC
ATGAGCCAATCCTGGAAGAACCAGCAGATAGACACCCAAATCGTAAAGCAAGAGGACAGCTACG
GACCAAAATAGAGTCTGGIGAGGGGACGATTCCAGTGCGCTCCAATGCGAGCATCCAAACGTGG
GACGGGGTGCTGCAAGGGGAGCGGCTGCTCACCATGTCCTGCAGTGACAAGATTGCACGCTGGA
ACGTGGTGGGCATCCAGGGATCCCTGCTCAGCATTTTCGTGGAGCCCATTTACTTCTCGAGCAT
CATCCTGGGCAGCCTTTACCACGGGGACCACCTTTCCAGGGCCATGTACCAGCGGATCTCCAAC
ATAGAGGACCTGCCACCTCTCTACACCCTCAACAAGCCTTTGCTCAGTGGCATCAGCAATGCAG
AAGCACGGCAGCCAGGGAAGGCCCCCAACTTCAGTGTCAACTGGACGGTAGGCGACTCCGCTAT
TGAGGTCATCAACGCCACGACTGGGAAGGATGAGCTGGGCCGCGCGTCCCGCCTGTGTAAGCAC
GCGTTGTACTGTCGCTGGATGCGTGTGCACGGCAAGGTTCCCTCCCACTTACTACGCTCCAAGA
TTACCAAGCCCAACGTGTACCATGAGTCCAAGCTGGCGGCAAAGGAGTACCAGGCCGCCAAGGC
GCGTCTGTTCACAGCCTTCATCAAGGCGGGGCTGGGGGCCTGGGTGGAGAAGCCCACCGAGCAG
GACCAGTTCTCACTCACGCCCTGA
SEQ ID NO: 56
ATGGAACAAAAACTTATTTCTGAAGAAGATCTGAATCCGCGGCAGGGGTATTCCCTCAGCGGAT
ACTACACCCATCCATTTCAAGGCTATGAGCACAGACAGCTCAGGTACCAGCAGCCTGGGCCAGG
ATCTTCCCCCAGTAGTTTCCTGCTTAAGCAAATAGAATTTCTCAAGGGGCAGCTCCCAGAAGCA
CCGGTGATTGGAAAGCAGACACCGTCACTGCCACCTTCCCTCCCAGGACTCCGGCCAAGGTTTC
CAGTACTACTTGCCTCCAGTACCAGAGGCAGGCAAGTGGACATCAGGGGTGTCCCCAGGGGCGT
GCATCTCAGAAGTCAGGGGCTCCAGAGAGGGTTCCAGCATCCTTCACCACGTGGCAGGAGTCTG
CCACAGAGAGGTGTTGATTGCCTTTCCTCACATTTCCAGGAACTGAGTATCTACCAAGATCAGG
AACAAAGGATCTTAAAGTTCCTGGAAGAGCTTGGGGAAGGGAAGGCCACCACAGCACATGATCT
GTCTGGGAAACTTGGGACTCCGAAGAAAGAAATCAATCGAGTTTTATACTCCCTGGCAAAGAAG
GGCAAGCTACAGAAAGAGGCAGGAACACCCCCTTTGTGGAAAATCGCGGTCTCCACTCAGGCTT
GGAACCAGCACAGCGGAGTGGTAAGACCAGACGGTCATAGCCAAGGAGCCCCAAACTCAGACCC
GAGTTTGGAACCGGAAGACAGAAACTCCACATCTGTCTCAGAAGATCTTCTTGAGCCTTTTATT
GCAGTCTCAGCTCAGGCTTGGAACCAGCACAGCGGAGTGGTAAGACCAGACAGTCATAGCCAAG
GATCCCCAAACTCAGACCCAGGTTTGGAACCTGAAGACAGCAACTCCACATCTGCCTTGGAAGA
TCCTCTTGAGTTTTTAGACATGGCCGAGATCAAGGAGAAAATCTGCGACTATCTCTTCAATGTG
TCTGACTCCTCTGCCCTGAATTTGGCTAAAAATATTGGCCTTACCAAGGCCCGAGATATAAATG
CTGTGCTAATTGACATGGAAAGGCAGGGGGATGTCTATAGACAAGGGACAACCCCTCCCATATG
GCATTTGACAGACAAGAAGCGAGAGAGGATGCAAATCAAGAGAAATACGAACAGTGTTCCTGAA
ACCGCTCCAGCTGCAATCCCTGAGACCAAAAGAAACGCAGAGTTCCTCACCTGTAATATACCCA
CATCAAATGCCTCAAATAACATGGIAACCACAGAAAAAGTGGAGAATGGGCAGGAACCTGTCAT
AAAGTTAGAAAACAGGCAAGAGGCCAGACCAGAACCAGCAAGACTGAAACCACCTGTTCATTAC
AATGGCCCCTCAAAAGCAGGGTATGTTGACTTTGAAAATGGCCAGTGGGCCACAGATGACATCC
CAGATGACTTGAATAGTATCCGCGCAGCACCAGGTGAGTTTCGAGCCATCATGGAGATGCCCTC
CTTCTACAGTCATGGCTTGCCACGGTGTTCACCCTACAAGAAACTGACAGAGTGCCAGCTGAAG
AACCCCATCAGCGGGCTGTTAGAATATGCCCAGTTCGCTAGTCAAACCTGTGAGTTCAACATGA
TAGAGCAGAGTGGACCACCCCATGAACCTCGATTTAAATTCCAGGTTGTCATCAATGGCCGAGA
GTTTCCCCCAGCTGAAGCTGGAAGCAAGAAAGTGGCCAAGCAGGATGCAGCTATGAAAGCCATG
ACAATTCTGCTAGAGGAAGCCAAAGCCAAGGACAGTGGAAAATCAGAAGAATCATCCCACTATT
CCACAGAGAAAGAATCAGAGAAGACTGCAGAGTCCCAGACCCCCACCCCTTCAGCCACATCCTT
CTTTTCTGGGAAGAGCCCCGTCACCACACTGCTTGAGTGTATGCACAAATTGGGGAACTCCTGC
GAATTCCGTCTCCTGTCCAAAGAAGGCCCTGCCCATGAACCCAAGTTCCAATACTGTGTTGCAG
TGGGAGCCCAAACTTTCCCCAGTGTGAGTGCTCCCAGCAAGAAAGTGGCAAAGCAGATGGCCGC
AGAGGAAGCCATGAAGGCCCTGCATGGGGAGGCGACCAACTCCATGGCTTCTGATAACCAGCCT
GAAGGTATGATCTCAGAGTCACTTGATAACTTGGAATCCATGATGCCCAACAAGGTCAGGAAGA
TTGGCGAGCTCGTGAGATACCTGAACACCAACCCTGTGGGTGGCCTTTTGGAGTACGCCCGCTC
CCATGGCTTTGCTGCTGAATTCAAGTTGGTCGACCAGTCCGGACCTCCTCACGAGCCCAAGTTC
GTTTACCAAGCAAAAGTTGGGGGTCGCTGGTTCCCAGCCGTCTGCGCACACAGCAAGAAGCAAG
GCAAGCAGGAAGCAGCAGATGCGGCTCTCCGTGTCTTGATTGGGGAGAACGAGAAGGCAGAACG
CATGGGTTTCACAGAGGIAACCCCAGTGACAGGGGCCAGTCTCAGAAGAACTATGCTCCTCCTC
TCAAGGTCCCCAGAAGCACAGCCAAAGACACTCCCTCTCACTGGCAGCACCTTCCATGACCAGA
TAGCCATGCTGAGCCACCGGTGCTTCAACACTCTGACTAACAGCTTCCAGCCCTCCTTGCTCGG
CCGCAAGATTCTGGCCGCCATCATTATGAAAAAAGACTCTGAGGACATGGGTGTCGTCGTCAGC
TTGGGAACAGGGAATCGCTGTGTGAAAGGAGATTCTCTCAGCCTAAAAGGAGAAACTGTCAATG
ACTGCCATGCAGAAATAATCTCCCGGAGAGGCTTCATCAGGTTTCTCTACAGTGAGTTAATGAA
ATACAACTCCCAGACTGCGAAGGATAGTATATTTGAACCTGCTAAGGGAGGAGAAAAGCTCCAA
ATAAAAAAGACTGTGTCATTCCATCTGTATATCAGCACTGCTCCGTGTGGAGATGGCGCCCTCT
TTGACAAGTCCTGCAGCGACCGTGCTATGGAAAGCACAGAATCCCGCCACTACCCTGTCTTCGA
GAATCCCAAACAAGGAAAGCTCCGCACCAAGGTGGAGAACGGAGAAGGCACAATCCCTGTGGAA
TCCAGTGACATTGTGCCTACGTGGGATGGCATTCGGCTCGGGGAGAGACTCCGTACCATGTCCT
GTAGTGACAAAATCCTACGCTGGAACGTGCTGGGCCTGCAAGGGGCACTGTTGACCCACTTCCT
GCAGCCCATTTATCTCAAATCTGTCACATTGGGTTACCTTTTCAGCCAAGGGCATCTGACCCGT
GCTATTTGCTGTCGTGTGACAAGAGATGGGAGTGCATTTGAGGATGGACTACGACATCCCTTTA
TTGTCAACCACCCCAAGGTTGGCAGAGTCAGCATATATGATTCCAAAAGGCAATCCGGGAAGAC
TAAGGAGACAAGCGTCAACTGGTGTCTGGCTGATGGCTATGACCTGGAGATCCTGGACGGTACC
AGAGGCACTGTGGATGGGCCACGGAATGAATTGTCCCGGGTCTCCAAAAAGAACATTTTTCTTC
TATTTAAGAAGCTCTGCTCCTTCCGTTACCGCAGGGATCTACTGAGACTCTCCTATGGTGAGGC
CAAGAAAGCTGCCCGTGACTACGAGACGGCCAAGAACTACTTCAAAAAAGGCCTGAAGGATATG
GGCTATGGGAACTGGATTAGCAAACCCCAGGAGGAAAAGAACTTTTATCTCTGCCCAGTATAG
Homo sapiens methyl-CpG binding protein 2 (MECP2), NM_004992
SEQ ID NO: 57
CCGGCGTCGGCGGCGCGCGCGCTCCCTCCTCTCGGAGAGAGGGCTGTGGTAAAAGCCGTCCGGA
AAATGGCCGCCGCCGCCGCCGCCGCGCCGAGCGGAGGAGGAGGAGGAGGCGAGGAGGAGAGACT
GCTCCATAAAAATACAGACTCACCAGTTCCTGCTTTGATGTGACATGTGACTCCCCAGAATACA
CCTTGCTTCTGTAGACCAGCTCCAACAGGATTCCATGGTAGCTGGGATGTTAGGGCTCAGGGAA
GAAAAGTCAGAAGACCAGGACCTCCAGGGCCTCAAGGACAAACCCCTCAAGTTTAAAAAGGTGA
AGAAAGATAAGAAAGAAGAGAAAGAGGGCAAGCATGAGCCCGTGCAGCCATCAGCCCACCACTC
TGCTGAGCCCGCAGAGGCAGGCAAAGCAGAGACATCAGAAGGGTCAGGCTCCGCCCCGGCTGTG
CCGGAAGCTTCTGCCTCCCCCAAACAGCGGCGCTCCATCATCCGTGACCGGGGACCCATGTATG
ATGACCCCACCCTGCCTGAAGGCTGGACACGGAAGCTTAAGCAAAGGAAATCTGGCCGCTCTGC
TGGGAAGTATGATGTGTATTTGATCAATCCCCAGGGAAAAGCCTTTCGCTCTAAAGTGGAGTTG
ATTGCGTACTTCGAAAAGGTAGGCGACACATCCCTGGACCCTAATGATTTTGACTTCACGGTAA
CTGGGAGAGGGAGCCCCTCCCGGCGAGAGCAGAAACCACCTAAGAAGCCCAAATCTCCCAAAGC
TCCAGGAACTGGCAGAGGCCGGGGACGCCCCAAAGGGAGCGGCACCACGAGACCCAAGGCGGCC
ACGTCAGAGGGTGTGCAGGTGAAAAGGGTCCTGGAGAAAAGTCCTGGGAAGCTCCTTGTCAAGA
TGCCTTTTCAAACTTCGCCAGGGGGCAAGGCTGAGGGGGGTGGGGCCACCACATCCACCCAGGT
CATGGTGATCAAACGCCCCGGCAGGAAGCGAAAAGCTGAGGCCGACCCTCAGGCCATTCCCAAG
AAACGGGGCCGAAAGCCGGGGAGTGTGGTGGCAGCCGCTGCCGCCGAGGCCAAAAAGAAAGCCG
TGAAGGAGTCTTCTATCCGATCTGTGCAGGAGACCGTACTCCCCATCAAGAAGCGCAAGACCCG
GGAGACGGTCAGCATCGAGGTCAAGGAAGTGGTGAAGCCCCTGCTGGTGTCCACCCTCGGTGAG
AAGAGCGGGAAAGGACTGAAGACCTGTAAGAGCCCTGGGCGGAAAAGCAAGGAGAGCAGCCCCA
AGGGGCGCAGCAGCAGCGCCTCCTCACCCCCCAAGAAGGAGCACCACCACCATCACCACCACTC
AGAGTCCCCAAAGGCCCCCGTGCCACTGCTCCCACCCCTGCCCCCACCTCCACCTGAGCCCGAG
AGCTCCGAGGACCCCACCAGCCCCCCTGAGCCCCAGGACTTGAGCAGCAGCGTCTGCAAAGAGG
AGAAGATGCCCAGAGGAGGCTCACTGGAGAGCGACGGCTGCCCCAAGGAGCCAGCTAAGACTCA
GCCCGCGGTTGCCACCGCCGCCACGGCCGCAGAAAAGTACAAACACCGAGGGGAGGGAGAGCGC
AAAGACATTGTTTCATCCTCCATGCCAAGGCCAAACAGAGAGGAGCCTGTGGACAGCCGGACGC
CCGTGACCGAGAGAGTTAGCTGACTTTACACGGAGCGGATTGCAAAGCAAACCAACAAGAATAA
AGGCAGCTGTTGTCTCTTCTCCTTATGGGTAGGGCTCTGACAAAGCTTCCCGATTAACTGAAAT
AAAAAATATTTTTTTTTCTTTCAGTAAACTTAGAGTTTCGTGGCTTCAGGGTGGGAGTAGTTGG
AGCATTGGGGATGTTTTTCTTACCGACAAGCACAGTCAGGTTGAAGACCTAACCAGGGCCAGAA
GTAGCTTTGCACTTTTCTAAACTAGGCTCCTTCAACAAGGCTTGCTGCAGATACTACTGACCAG
ACAAGCTGTTGACCAGGCACCTCCCCTCCCGCCCAAACCTTTCCCCCATGTGGTCGTTAGAGAC
AGAGCGACAGAGCAGTTGAGAGGACACTCCCGTTTTCGGTGCCATCAGTGCCCCGTCTACAGCT
CCCCCAGCTCCCCCCACCTCCCCCACTCCCAACCACGTTGGGACAGGGAGGTGTGAGGCAGGAG
AGACAGTTGGATTCTTTAGAGAAGATGGATATGACCAGTGGCTATGGCCTGTGCGATCCCACCC
GTGGTGGCTCAAGTCTGGCCCCACACCAGCCCCAATCCAAAACTGGCAAGGACGCTTCACAGGA
CAGGAAAGTGGCACCTGTCTGCTCCAGCTCTGGCATGGCTAGGAGGGGGGAGTCCCTTGAACTA
CTGGGTGTAGACTGGCCTGAACCACAGGAGAGGATGGCCCAGGGTGAGGTGGCATGGTCCATTC
TCAAGGGACGTCCTCCAACGGGTGGCGCTAGAGGCCATGGAGGCAGTAGGACAAGGTGCAGGCA
GGCTGGCCTGGGGTCAGGCCGGGCAGAGCACAGCGGGGTGAGAGGGATTCCTAATCACTCAGAG
CAGTCTGTGACTTAGTGGACAGGGGAGGGGGCAAAGGGGGAGGAGAAGAAAATGTTCTTCCAGT
TACTTTCCAATTCTCCTTTAGGGACAGCTTAGAATTATTTGCACTATTGAGTCTTCATGTTCCC
ACTTCAAAACAAACAGATGCTCTGAGAGCAAACTGGCTTGAATTGGTGACATTTAGTCCCTCAA
GCCACCAGATGTGACAGTGTTGAGAACTACCTGGATTTGTATATATACCTGCGCTTGTTTTAAA
GTGGGCTCAGCACATAGGGTTCCCACGAAGCTCCGAAACTCTAAGTGTTTGCTGCAATTTTATA
AGGACTTCCTGATTGGTTTCTCTTCTCCCCTTCCATTTCTGCCTTTTGTTCATTTCATCCTTTC
ACTTCTTTCCCTTCCTCCGTCCTCCTCCTTCCTAGTTCATCCCTTCTCTTCCAGGCAGCCGCGG
TGCCCAACCACACTTGTCGGCTCCAGTCCCCAGAACTCTGCCTGCCCTTTGTCCTCCTGCTGCC
AGTACCAGCCCCACCCTGTTTTGAGCCCTGAGGAGGCCTTGGGCTCTGCTGAGTCCGACCTGGC
CTGTCTGTGAAGAGCAAGAGAGCAGCAAGGTCTTGCTCTCCTAGGTAGCCCCCTCTTCCCTGGT
AAGAAAAAGCAAAAGGCATTTCCCACCCTGAACAACGAGCCTTTTCACCCTTCTACTCTAGAGA
AGTGGACTGGAGGAGCTGGGCCCGATTTGGTAGTTGAGGAAAGCACAGAGGCCTCCTGTGGCCT
GCCAGTCATCGAGTGGCCCAACAGGGGCTCCATGCCAGCCGACCTTGACCTCACTCAGAAGTCC
AGAGTCTAGCGTAGTGCAGCAGGGCAGTAGCGGTACCAATGCAGAACTCCCAAGACCCGAGCTG
GGACCAGTACCTGGGTCCCCAGCCCTTCCTCTGCTCCCCCTTTTCCCTCGGAGTTCTTCTTGAA
TGGCAATGTTTTGCTTTTGCTCGATGCAGACAGGGGGCCAGAACACCACACATTTCACTGTCTG
TCTGGTCCATAGCTGTGGTGTAGGGGCTTAGAGGCATGGGCTTGCTGTGGGTTTTTAATTGATC
AGTTTTCATGTGGGATCCCATCTTTTTAACCTCTGTTCAGGAAGTCCTTATCTAGCTGCATATC
TTCATCATATTGGTATATCCTTTTCTGTGTTTACAGAGATGTCTCTTATATCTAAATCTGTCCA
ACTGAGAAGTACCTTATCAAAGTAGCAAATGAGACAGCAGTCTTATGCTTCCAGAAACACCCAC
AGGCATGTCCCATGTGAGCTGCTGCCATGAACTGTCAAGTGTGTGTTGTCTTGTGTATTTCAGT
TATTGTCCCTGGCTTCCTTACTATGGTGTAATCATGAAGGAGTGAAACATCATAGAAACTGTCT
AGCACTTCCTTGCCAGTCTTTAGTGATCAGGAACCATAGTTGACAGTTCCAATCAGTAGCTTAA
GAAAAAACCGTGTTTGTCTCTTCTGGAATGGTTAGAAGTGAGGGAGTTTGCCCCGTTCTGTTTG
TAGAGTCTCATAGTTGGACTTTCTAGCATATATGTGTCCATTTCCTTATGCTGTAAAAGCAAGT
CCTGCAACCAAACTCCCATCAGCCCAATCCCTGATCCCTGATCCCTTCCACCTGCTCTGCTGAT
GACCCCCCCAGCTTCACTTCTGACTCTTCCCCAGGAAGGGAAGGGGGGTCAGAAGAGAGGGTGA
GTCCTCCAGAACTCTTCCTCCAAGGACAGAAGGCTCCTGCCCCCATAGTGGCCTCGAACTCCTG
GCACTACCAAAGGACACTTATCCACGAGAGCGCAGCATCCGACCAGGTTGTCACTGAGAAGATG
TTTATTTTGGTCAGTTGGGTTTTTATGTATTATACTTAGTCAAATGTAATGTGGCTTCTGGAAT
CATTGTCCAGAGCTGCTTCCCCGTCACCTGGGCGTCATCTGGTCCTGGTAAGAGGAGTGCGTGG
CCCACCAGGCCCCCCTGTCACCCATGACAGTTCATTCAGGGCCGATGGGGCAGTCGTGGTTGGG
AACACAGCATTTCAAGCGTCACTTTATTTCATTCGGGCCCCACCTGCAGCTCCCTCAAAGAGGC
AGTTGCCCAGCCTCTTTCCCTTCCAGTTTATTCCAGAGCTGCCAGTGGGGCCTGAGGCTCCTTA
GGGTTTTCTCTCTATTTCCCCCTTTCTTCCTCATTCCCTCGTCTTTCCCAAAGGCATCACGAGT
CAGTCGCCTTTCAGCAGGCAGCCTTGGCGGTTTATCGCCCTGGCAGGCAGGGGCCCTGCAGCTC
TCATGCTGCCCCTGCCTTGGGGTCAGGTTGACAGGAGGTTGGAGGGAAAGCCTTAAGCTGCAGG
ATTCTCACCAGCTGIGTCCGGCCCAGTTTTGGGGTGTGACCTCAATTTCAATTTIGTCTGTACT
TGAACATTATGAAGATGGGGGCCTCTTTCAGTGAATTTGTGAACAGCAGAATTGACCGACAGCT
TTCCAGTACCCATGGGGCTAGGTCATTAAGGCCACATCCACAGTCTCCCCCACCCTTGTTCCAG
TTGTTAGTTACTACCTCCTCTCCTGACAATACTGTATGTCGTCGAGCTCCCCCCAGGTCTACCC
CTCCCGGCCCTGCCTGCTGGTGGGCTTGTCATAGCCAGTGGGATTGCCGGTCTTGACAGCTCAG
TGAGCTGGAGATACTTGGTCACAGCCAGGCGCTAGCACAGCTCCCTTCTGTTGATGCTGTATTC
CCATATCAAAAGACACAGGGGACACCCAGAAACGCCACATCCCCCAATCCATCAGTGCCAAACT
AGCCAACGGCCCCAGCTTCTCAGCTCGCTGGATGGCGGAAGCTGCTACTCGTGAGCGCCAGTGC
GGGTGCAGACAATCTTCTGTTGGGTGGCATCATTCCAGGCCCGAAGCATGAACAGTGCACCTGG
GACAGGGAGCAGCCCCAAATTGTCACCTGCTTCTCTGCCCAGCTTTTCATTGCTGTGACAGTGA
TGGCGAAAGAGGGTAATAACCAGACACAAACTGCCAAGTTGGGTGGAGAAAGGAGTTTCTTTAG
CTGACAGAATCTCTGAATTTTAAATCACTTAGTAAGCGGCTCAAGCCCAGGAGGGAGCAGAGGG
ATACGAGCGGAGTCCCCTGCGCGGGACCATCTGGAATTGGTTTAGCCCAAGTGGAGCCTGACAG
CCAGAACTCTGTGTCCCCCGTCTAACCACAGCTCCTTTTCCAGAGCATTCCAGTCAGGCTCTCT
GGGCTGACTGGGCCAGGGGAGGTTACAGGTACCAGTTCTTTAAGAAGATCTTTGGGCATATACA
TTTTTAGCCTGTGTCATTGCCCCAAATGGATTCCTGTTTCAAGTTCACACCTGCAGATTCTAGG
ACCTGTGTCCTAGACTTCAGGGAGTCAGCTGTTTCTAGAGTTCCTACCATGGAGTGGGTCTGGA
GGACCTGCCCGGTGGGGGGGCAGAGCCCTGCTCCCTCCGGGTCTTCCTACTCTTCTCTCTGCTC
TGACGGGATTTGTTGATTCTCTCCATTTTGGTGTCTTTCTCTTTTAGATATTGTATCAATCTTT
AGAAAAGGCATAGTCTACTTGTTATAAATCGTTAGGATACTGCCTCCCCCAGGGTCTAAAATTA
CATATTAGAGGGGAAAAGCTGAACACTGAAGTCAGTTCTCAACAATTTAGAAGGAAAACCTAGA
AAACATTTGGCAGAAAATTACATTTCGATGTTTTTGAATGAATACGAGCAAGCTTTTACAACAG
TGCTGATCTAAAAATACTTAGCACTTGGCCTGAGATGCCTGGTGAGCATTACAGGCAAGGGGAA
TCTGGAGGTAGCCGACCTGAGGACATGGCTTCTGAACCTGTCTTTTGGGAGTGGTATGGAAGGT
GGAGCGTTCACCAGTGACCTGGAAGGCCCAGCACCACCCTCCTTCCCACTCTTCTCATCTTGAC
AGAGCCTGCCCCAGCGCTGACGTGTCAGGAAAACACCCAGGGAACTAGGAAGGCACTTCTGCCT
GAGGGGCAGCCTGCCTTGCCCACTCCTGCTCTGCTCGCCTCGGATCAGCTGAGCCTTCTGAGCT
GGCCTCTCACTGCCTCCCCAAGGCCCCCTGCCTGCCCTGTCAGGAGGCAGAAGGAAGCAGGTGT
GAGGGCAGTGCAAGGAGGGAGCACAACCCCCAGCTCCCGCTCCGGGCTCCGACTTGTGCACAGG
CAGAGCCCAGACCCTGGAGGAAATCCTACCTTTGAATTCAAGAACATTTGGGGAATTTGGAAAT
CTCTTTGCCCCCAAACCCCCATTCTGTCCTACCTTTAATCAGGTCCTGCTCAGCAGTGAGAGCA
GATGAGGTGAAAAGGCCAAGAGGTTTGGCTCCTGCCCACTGATAGCCCCTCTCCCCGCAGTGTT
TGTGTGTCAAGTGGCAAAGCTGTTCTTCCTGGTGACCCTGATTATATCCAGTAACACATAGACT
GTGCGCATAGGCCTGCTTTGTCTCCTCTATCCTGGGCTTTTGTTTTGCTTTTTAGTTTTGCTTT
TAGTTTTTCTGTCCCTTTTATTTAACGCACCGACTAGACACACAAAGCAGTTGAATTTTTATAT
ATATATCTGTATATTGCACAATTATAAACTCATTTTGCTTGTGGCTCCACACACACAAAAAAAG
ACCTGTTAAAATTATACCTGTTGCTTAATTACAATATTTCTGATAACCATAGCATAGGACAAGG
GAAAATAAAAAAAGAAAAAAAAGAAAAAAAAACGACAAATCTGTCTGCTGGTCACTTCTTCTGT
CCAAGCAGATTCGTGGTCTTTTCCTCGCTTCTTTCAAGGGCTTTCCTGTGCCAGGTGAAGGAGG
CTCCAGGCAGCACCCAGGTTTTGCACTCTTGTTTCTCCCGTGCTTGTGAAAGAGGTCCCAAGGT
TCTGGGTGCAGGAGCGCTCCCTTGACCTGCTGAAGTCCGGAACGTAGTCGGCACAGCCTGGTCG
CCTTCCACCTCTGGGAGCTGGAGTCCACTGGGGTGGCCTGACTCCCCCAGTCCCCTTCCCGTGA
CCTGGTCAGGGTGAGCCCATGTGGAGTCAGCCTCGCAGGCCTCCCTGCCAGTAGGGTCCGAGTG
TGTTTCATCCTTCCCACTCTGTCGAGCCTGGGGGCTGGAGCGGAGACGGGAGGCCTGGCCTGTC
TCGGAACCTGTGAGCTGCACCAGGTAGAACGCCAGGGACCCCAGAATCATGTGCGTCAGTCCAA
GGGGTCCCCTCCAGGAGTAGTGAAGACTCCAGAAATGTCCCTTTCTTCTCCCCCATCCTACGAG
TAATTGCATTTGCTTTTGTAATTCTTAATGAGCAATATCTGCTAGAGAGTTTAGCTGTAACAGT
TCTTTTTGATCATCTTTTTTTAATAATTAGAAACACCAAAAAAATCCAGAAACTTGTTCTTCCA
AAGCAGAGAGCATTATAATCACCAGGGCCAAAAGCTTCCCTCCCTGCTGTCATTGCTTCTTCTG
AGGCCTGAATCCAAAAGAAAAACAGCCATAGGCCCTTTCAGTGGCCGGGCTACCCGTGAGCCCT
TCGGAGGACCAGGGCTGGGGCAGCCTCTGGGCCCACATCCGGGGCCAGCTCCGGCGTGTGTTCA
GTGTTAGCAGTGGGTCATGATGCTCTTTCCCACCCAGCCTGGGATAGGGGCAGAGGAGGCGAGG
AGGCCGTTGCCGCTGATGTTTGGCCGTGAACAGGTGGGTGTCTGCGTGCGTCCACGTGCGTGTT
TTCTGACTGACATGAAATCGACGCCCGAGTTAGCCTCACCCGGTGACCTCTAGCCCTGCCCGGA
TGGAGCGGGGCCCACCCGGTTCAGTGTTTCTGGGGAGCTGGACAGTGGAGTGCAAAAGGCTTGC
AGAACTTGAAGCCTGCTCCTTCCCTTGCTACCACGGCCTCCTTTCCGTTTGATTTGTCACTGCT
TCAATCAATAACAGCCGCTCCAGAGTCAGTAGTCAATGAATATATGACCAAATATCACCAGGAC
TGTTACTCAATGIGTGCCGAGCCCTTGCCCATGCTGGGCTCCCGTGTATCTGGACACTGTAACG
TGTGCTGTGTTTGCTCCCCTTCCCCTTCCTTCTTTGCCCTTTACTTGTCTTTCIGGGGTTTTTC
TGTTTGGGTTTGGTTTGGTTTTTATTTCTCCTTTTGTGTTCCAAACATGAGGTTCTCTCTACTG
GTCCTCTTAACTGTGGTGTTGAGGCTTATATTTGTGTAATTTTTGGTGGGTGAAAGGAATTTTG
CTAAGTAAATCTCTTCTGTGTTTGAACTGAAGTCTGTATTGTAACTATGTTTAAAGTAATTGTT
CCAGAGACAAATATTTCTAGACACTTTTTCTTTACAAACAAAAGCATTCGGAGGGAGGGGGATG
GTGACTGAGATGAGAGGGGAGAGCTGAACAGATGACCCCTGCCCAGATCAGCCAGAAGCCACCC
AAAGCAGTGGAGCCCAGGAGTCCCACTCCAAGCCAGCAAGCCGAATAGCTGATGTGTTGCCACT
TTCCAAGTCACTGCAAAACCAGGTTTTGTTCCGCCCAGTGGATTCTTGTTTTGCTTCCCCTCCC
CCCGAGATTATTACCACCATCCCGTGCTTTTAAGGAAAGGCAAGATTGATGTTTCCTTGAGGGG
AGCCAGGAGGGGATGTGTGTGTGCAGAGCTGAAGAGCTGGGGAGAATGGGGCTGGGCCCACCCA
AGCAGGAGGCTGGGACGCTCTGCTGTGGGCACAGGTCAGGCTAATGTTGGCAGATGCAGCTCTT
CCTGGACAGGCCAGGTGGTGGGCATTCTCTCTCCAAGGTGTGCCCCGTGGGCATTACTGTTTAA
GACACTTCCGTCACATCCCACCCCATCCTCCAGGGCTCAACACTGTGACATCTCTATTCCCCAC
CCTCCCCTTCCCAGGGCAATAAAATGACCATGGAGGGGGCTTGCACTCTCTTGGCTGTCACCCG
ATCGCCAGCAAAACTTAGATGIGAGAAAACCCCTTCCCATTCCATGGCGAAAACATCTCCTTAG
AAAAGCCATTACCCTCATTAGGCATGGTTTTGGGCTCCCAAAACACCTGACAGCCCCTCCCTCC
TCTGAGAGGCGGAGAGTGCTGACTGTAGTGACCATTGCATGCCGGGTGCAGCATCTGGAAGAGC
TAGGCAGGGTGTCTGCCCCCTCCTGAGTTGAAGTCATGCTCCCCTGTGCCAGCCCAGAGGCCGA
GAGCTATGGACAGCATTGCCAGTAACACAGGCCACCCTGTGCAGAAGGGAGCTGGCTCCAGCCT
GGAAACCTGTCTGAGGTTGGGAGAGGTGCACTTGGGGCACAGGGAGAGGCCGGGACACACTTAG
CTGGAGATGTCTCTAAAAGCCCTGTATCGTATTCACCTTCAGTTTTTGTGTTTTGGGACAATTA
CTTTAGAAAATAAGTAGGTCGTTTTAAAAACAAAAATTATTGATTGCTTTTTTGTAGTGTTCAG
AAAAAAGGTTCTTTGTGTATAGCCAAATGACTGAAAGCACTGATATATTTAAAAACAAAAGGCA
ATTTATTAAGGAAATTTGTACCATTTCAGTAAACCTGTCTGAATGTACCTGTATACGTTTCAAA
AACACCCCCCCCCCACTGAATCCCTGTAACCTATTTATTATATAAAGAGTTTGCCTTATAAATT
T
Homo sapiens MECP2 R255X
SEQ ID NO: 58
ATGTACCCATACGACGTCCCAGACTACGCTGTAGCTGGGATGTTAGGGCTCAGGGAAGAAAAGT
CAGAAGACCAGGACCTCCAGGGCCTCAAGGACAAACCCCTCAAGTTTAAAAAGGTGAAGAAAGA
TAAGAAAGAAGAGAAAGAGGGCAAGCATGAGCCCGTGCAGCCATCAGCCCACCACTCTGCTGAG
CCCGCAGAGGCAGGCAAAGCAGAGACATCAGAAGGGTCAGGCTCCGCCCCGGCTGTGCCGGAAG
CTTCTGCCTCCCCCAAACAGCGGCGCTCCATCATCCGTGACCGGGGACCCATGTATGATGACCC
CACCCTGCCTGAAGGCTGGACACGGAAGCTTAAGCAAAGGAAATCTGGCCGCTCTGCTGGGAAG
TATGATGIGTATTTGATCAATCCCCAGGGAAAAGCCTTTCGCTCTAAAGTGGAGTTGATTGCGT
ACTTCGAAAAGGTAGGCGACACATCCCTGGACCCTAATGATTTTGACTTCACGGTAACTGGGAG
AGGGAGCCCCTCCCGGCGAGAGCAGAAACCACCTAAGAAGCCCAAATCTCCCAAAGCTCCAGGA
ACTGGCAGAGGCCGGGGACGCCCCAAAGGGAGCGGCACCACGAGACCCAAGGCGGCCACGTCAG
AGGGTGTGCAGGTGAAAAGGGTCCTGGAGAAAAGTCCTGGGAAGCTCCTTGTCAAGATGCCTTT
TCAAACTTCGCCAGGGGGCAAGGCTGAGGGGGGTGGGGCCACCACATCCACCCAGGTCATGGTG
ATCAAACGCCCCGGCAGGAAGTGAAAAGCTGAGGCCGACCCTCAGGCCATTCCCAAGAAACGGG
GCCGAAAGCCGGGGAGTGTGGTGGCAGCCGCTGCCGCCGAGGCCAAAAAGAAAGCCGTGAAGGA
GTCTTCTATCCGATCTGTGCAGGAGACCGTACTCCCCATCAAGAAGCGCAAGACCCGGGAGACG
GTCAGCATCGAGGTCAAGGAAGTGGTGAAGCCCCTGCTGGTGTCCACCCTCGGTGAGAAGAGCG
GGAAAGGACTGAAGACCTGTAAGAGCCCTGGGCGGAAAAGCAAGGAGAGCAGCCCCAAGGGGCG
CAGCAGCAGCGCCTCCTCACCCCCCAAGAAGGAGCACCACCACCATCACCACCACTCAGAGTCC
CCAAAGGCCCCCGTGCCACTGCTCCCACCCCTGCCCCCACCTCCACCTGAGCCCGAGAGCTCCG
AGGACCCCACCAGCCCCCCTGAGCCCCAGGACTTGAGCAGCAGCGTCTGCAAAGAGGAGAAGAT
GCCCAGAGGAGGCTCACTGGAGAGCGACGGCTGCCCCAAGGAGCCAGCTAAGACTCAGCCCGCG
GTTGCCACCGCCGCCACGGCCGCAGAAAAGTACAAACACCGAGGGGAGGGAGAGCGCAAAGACA
TTGTTTCATCCTCCATGCCAAGGCCAAACAGAGAGGAGCCTGTGGACAGCCGGACGCCCGTGAC
CGAGAGAGTTAGCTGA,
Homo sapiens MECP2 R294X
SEQ ID NO: 59
ATGTACCCATACGACGTCCCAGACTACGCTGTAGCTGGGATGTTAGGGCTCAGGGAAGAAAAGT
CAGAAGACCAGGACCTCCAGGGCCTCAAGGACAAACCCCTCAAGTTTAAAAAGGTGAAGAAAGA
TAAGAAAGAAGAGAAAGAGGGCAAGCATGAGCCCGTGCAGCCATCAGCCCACCACTCTGCTGAG
CCCGCAGAGGCAGGCAAAGCAGAGACATCAGAAGGGTCAGGCTCCGCCCCGGCTGTGCCGGAAG
CTTCTGCCTCCCCCAAACAGCGGCGCTCCATCATCCGTGACCGGGGACCCATGTATGATGACCC
CACCCTGCCTGAAGGCTGGACACGGAAGCTTAAGCAAAGGAAATCTGGCCGCTCTGCTGGGAAG
TATGATGTGTATTTGATCAATCCCCAGGGAAAAGCCTTTCGCTCTAAAGTGGAGTTGATTGCGT
ACTTCGAAAAGGTAGGCGACACATCCCTGGACCCTAATGATTTTGACTTCACGGTAACTGGGAG
AGGGAGCCCCTCCCGGCGAGAGCAGAAACCACCTAAGAAGCCCAAATCTCCCAAAGCTCCAGGA
ACTGGCAGAGGCCGGGGACGCCCCAAAGGGAGCGGCACCACGAGACCCAAGGCGGCCACGTCAG
AGGGTGTGCAGGTGAAAAGGGTCCTGGAGAAAAGTCCTGGGAAGCTCCTTGTCAAGATGCCTTT
TCAAACTTCGCCAGGGGGCAAGGCTGAGGGGGGTGGGGCCACCACATCCACCCAGGTCATGGTG
ATCAAACGCCCCGGCAGGAAGCGAAAAGCTGAGGCCGACCCTCAGGCCATTCCCAAGAAACGGG
GCCGAAAGCCGGGGAGTGTGGTGGCAGCCGCTGCCGCCGAGGCCAAAAAGAAAGCCGTGAAGGA
GTCTTCTATCTGATCTGTGCAGGAGACCGTACTCCCCATCAAGAAGCGCAAGACCCGGGAGACG
GTCAGCATCGAGGTCAAGGAAGTGGTGAAGCCCCTGCTGGTGTCCACCCTCGGTGAGAAGAGCG
GGAAAGGACTGAAGACCTGTAAGAGCCCTGGGCGGAAAAGCAAGGAGAGCAGCCCCAAGGGGCG
CAGCAGCAGCGCCTCCTCACCCCCCAAGAAGGAGCACCACCACCATCACCACCACTCAGAGTCC
CCAAAGGCCCCCGTGCCACTGCTCCCACCCCTGCCCCCACCTCCACCTGAGCCCGAGAGCTCCG
AGGACCCCACCAGCCCCCCTGAGCCCCAGGACTTGAGCAGCAGCGTCTGCAAAGAGGAGAAGAT
GCCCAGAGGAGGCTCACTGGAGAGCGACGGCTGCCCCAAGGAGCCAGCTAAGACTCAGCCCGCG
GTTGCCACCGCCGCCACGGCCGCAGAAAAGTACAAACACCGAGGGGAGGGAGAGCGCAAAGACA
TTGTTTCATCCTCCATGCCAAGGCCAAACAGAGAGGAGCCTGTGGACAGCCGGACGCCCGTGAC
CGAGAGAGTTAGCTGA,
SEQ ID NO: 60
AGATGCCTTTCCAAGCATCGCC
SEQ ID NO: 61
TGGACACCAGCAGGGGCTTC
SEQ ID NO: 62
5′-
mG*mU*mG*mG*mAmAmUmAmGmUmAmUmAmAmCmAmAmUmAmUmGmCmUmAmAmAmUmGmUmU
mGmUmUmAmUmAmGmUmAmUmCmCmCmAmC-
mCmAmGmCmUmUdTdCdCmAmCmUmUmC*mC*mU*mG*mC-3′
SEQ ID NO: 63
5′-
mG*mU*mG*mG*mAmAmUmAmGmUmAmUmAmAmCmAmAmUmAmUmGmCmUmAmAmAmUmGmUmU
mGmUmUmAmUmAmGmUmAmUmCmCmCmAmC-
mCmAmGmCmUmUdTdCdCmAmCmUmUmCmCmUdG*mC*mC*mG-3′
SEQ ID NO: 64
5′-
mG*mG*mU*mG*mAmAmGmAmGmGmAmGmAmAmCmAmAmUmAmUmGmCmUmAmAmAmUmGmUmU
mGmUmUmCmUmCmGmUmCmUmCmCmAmCmC-
mCmAmGmCmUmUdTdCdCmAmCmUmUmC*mC*mU*mG*mC-3′
SEQ ID NO: 65
5′-
mG*mG*mU*mG*mUmCmGmAmGmAmAmGmAmGmGmAmGmAmAmCmAmAmUmAmUmGmCmUmAmA
mAmUmGmUmUmGmUmUmCmUmCmGmUmCmUmCmCmUmCmGmAmCmAmCmC-
mCmAmGmCmUmUdTdCdCmAmCmUmUmC*mC*mU*mG*mC-3′
SEQ ID NO: 66
5′-
mU*mU*mU*mC*mUmUmGmGmGmAmAmUmGmGmCmCmUmGmAmGmGmGmUmCmGmGmCmCmUmC
mAmGmCmUmUdTdCdCmAmCmUmUmCmCmUmG*mC*mC*mG*mG-3′
SEQ ID NO: 67
5′-
mU*mU*mU*mC*mUmUmGmGmGmAmAmUmGmGmCmCmUmGmAmGmGmGmUmCmGmGmCmCmUmC
mAmGmCmUmUmUdCdCmAmCmUmUmCmCmUmG*mC*mC*mG*mG-3′
SEQ ID NO: 68
5′-
mU*mU*mU*mC*mUmUmGmGmGmAmAmUmGmGmCmCmUmGmAmGmGmGmUmCmGmGmCmCmUmC
mAmGmCmUmUdTdCmCmAmCmUmUmCmCmUmG*mC*mC*mG*mG-3′
SEQ ID NO: 69
5′-
mU*mU*mU*mC*mUmUmGmGmGmAmAmUmGmGmCmCmUmGmAmGmAmGmUmCmAmGmCmCmUmC
mAmGmCmUmUdTdCdCmAmCmUmUmCmCmUmG*mC*mC*mG*mG-3′
SEQ ID NO: 70
5′-
mU*mU*mU*mC*mUmUmGmGmGmAmAmUmGmGmCmCmUmGmAmGmAmGmUmCmAmGmCmCmUmC
mAmGmCmUmUdGdCdCmAmCmUmUmCmCmUmG*mC*mC*mG*mG-3′
SEQ ID NO: 71
5′-
mU*mU*mU*mC*mUmUmGmGmGmAmAmUmGmGmCmCmUmGmCmGmGmGmCmCmGmGmCmCmUmC
mAmGmCmUmUdTdCdCmAmCmUmUmCmCmUmG*mC*mC*mG*mG-3′
SEQ ID NO: 72
5′-
mU*mU*mU*mC*mUmUmGmGmGmAmAmUmGmGmCmGmUmGmAmAmGmGmUmCmGmGmCmCmUmC
mAmGmCmUmUdTdCdCmAmCmUmUmCmCmUmG*mC*mC*mG*mG-3′
SEQ ID NO: 73
5′-
mU*mU*mU*mC*mUmUmGmGmGmAmAmUmGmGmCmGmUmGmAmAmGmGmUmCmGmGmCmCmUmC
mAmGmCmUmUdGdCdCmAmCmUmUmCmCmUmG*mC*mC*mG*mG-3′
SEQ ID NO: 74
5′-
mU*mU*mU*mC*mUmUmGmGmGmAmAmUmGmGmGmCmUmGmCmGmGmGmUmCmGmGmCmCmUmC
mAmGmCmUmUdTdCdCmAmCmUmUmCmCmUmG*mC*mC*mG*mG-3′
SEQ ID NO: 75
5′-
mU*mU*mU*mC*mUmUmGmGmGmAmAmUmAmGmCmCmCmGmAmGmGmGmUmCmGmGmCmCmUmC
mAmGmCmUmUdTdCdCmAmCmUmUmCmCmUmG*mC*mC*mG*mG-3′
SEQ ID NO: 76
5′-
mU*mU*mU*mC*mUmUmGmGmGmAmAmUmGmGmCmCmUmGmAmGmGmGmUmCmGmGmCmCmUmC
mAmGmCmUmUfUdCdCmAmCmUmUmCmCmUmG*mC*mC*mG*mG-3′
SEQ ID NO: 77
5′-
mU*mU*mU*mC*mUmUmGmGmGmAmAmUmGmGmCmCmUmGmAmGmGmGmUmCmGmGmCmCmUmC
mAmGmCmUmUdTfCdCmAmCmUmUmCmCmUmG*mC*mC*mG*mG-3′
SEQ ID NO: 78
5′-
mU*mU*mU*mC*mUmUmGmGmGmAmAmUmGmGmCmCmUmGmAmGmGmGmUmCmGmGmCmCmUmC
mAmGmCmUmUdTdCfCmAmCmUmUmCmCmUmG*mC*mC*mG*mG-3′
SEQ ID NO: 79
5′-
mU*mU*mU*mC*mUmUmGmGmGmAmAmUmGmGmCmCmUmGmAmGmGmGmUmCmGmGmCmCmUmC
mAmGmCmUmUfUfCfCmAmCmUmUmCmCmUmG*mC*mC*mG*mG-3′
SEQ ID NO: 80
5′-
mU*mU*mU*mC*mUmUmGmGmGmAmAmUmGmGmCmCmUmGmAmGmGmGmUmCmGmGmCmCmUmC
mAmGmCmUmUdTfCfCmAmCmUmUmCmCmUmG*mC*mC*mG*mG-3′
SEQ ID NO: 81
5′-
mU*mU*mU*mC*mUmUmGmGmGmAmAmUmGmGmCmCmUmGmAmGmGmGmUmCmGmGmCmCmUmC
mAmGmCmUmUfUfCdCmAmCmUmUmCmCmUmG*mC*mC*mG*mG-3′
SEQ ID NO: 82
5′-
mU*mU*mU*mC*mUmUmGmGmGmAmAmUmGmGmCmCmUmGmAmGmGmGmUmCmGmGmCmCmUmC
mAmGmCmUmUfUdCfCmAmCmUmUmCmCmUmG*mC*mC*mG*mG-3′
SEQ ID NO: 83
5′-
mG*mU*mG*mG*mAmAmUmAmGmUmAmUmAmAmCmAmAmUmAmUmGmCmUmAmAmAmUmGmUmU
mGmUmUmAmUmAmGmUmAmUmCmCmCmAmC-
mGmCmAmCmAmGdAdCdCmAmGmAmUmA*mG*mA*mA*mG-3′
SEQ ID NO: 84
5′-
mG*mU*mG*mG*mAmAmUmAmGmUmAmUmAmAmCmAmAmUmAmUmGmCmUmAmAmAmUmGmUmU
mGmUmUmAmUmAmGmUmAmUmCmCmCmAmC-
mGmCmAmCmAmGdAdCdCmAmGmAmUmAmGmA*mA*mG*mA*mC-3′
SEQ ID NO: 85
5′-
mG*mG*mU*mG*mAmAmGmAmGmGmAmGmAmAmCmAmAmUmAmUmGmCmUmAmAmAmUmGmUmU
mGmUmUmCmUmCmGmUmCmUmCmCmAmCmC-
mGmCmAmCmAmGdAdCdCmAmGmAmUmA*mG*mA*mA*mG-3′
SEQ ID NO: 86
5′-
mG*mG*mU*mG*mUmCmGmAmGmAmAmGmAmGmGmAmGmAmAmCmAmAmUmAmUmGmCmUmAmA
mAmUmGmUmUmGmUmUmCmUmCmGmUmCmUmCmCmUmCmGmAmCmAmCmC-
mGmCmAmCmAmGdAdCdCmAmGmAmUmA*mG*mA*mA*mG-3′
SEQ ID NO: 87
5′-
mG*mC*mG*mC*mUmUmCmUmUmGmAmUmGmGmGmGmAmGmUmAmCmGmGmUmCmUmCmCmUmG
mCmAmCmAmGdAdCdCmAmGmAmUmAmGmAmA*mG*mA*mC*mU-3′
SEQ ID NO: 88
5′-
mG*mC*mG*mC*mUmUmCmUmUmGmAmUmGmGmGmGmAmGmUmAmGmGmGmUmGmUmCmCmUmG
mCmAmCmAmGdAdCdCmAmGmAmUmAmGmAmA*mG*mA*mC*mU-3′
SEQ ID NO: 89
5′-
mG*mC*mG*mC*mUmUmCmUmUmGmAmUmGmGmGmGmAmGmUmCmCmGmGmCmCmUmCmCmUmG
mCmAmCmAmGdAdCdCmAmGmAmUmAmGmAmA*mG*mA*mC*mU-3′
SEQ ID NO: 90
5′-
mG*mC*mG*mC*mUmUmCmUmUmGmAmUmGmGmGmGmAmAmUmAmCmAmGmUmCmUmCmCmUmG
mCmAmCmAmGdAdCdCmAmGmAmUmAmGmAmA*mG*mA*mC*mU-3′
SEQ ID NO: 91
5′-
mG*mC*mG*mC*mUmUmCmUmUmGmAmUmGmGmGmAmAmGmUmCmCmGmGmUmCmUmCmCmUmG
mCmAmCmAmGdAdCdCmAmGmAmUmAmGmAmA*mG*mA*mC*mU-3′
SEQ ID NO: 92
5′-
mG*mC*mG*mC*mUmUmCmUmUmGmAmUmGmGmAmGmAmGmCmAmCmGmGmUmCmUmCmCmUmG
mCmAmCmAmGdAdCdCmAmGmAmUmAmGmAmA*mG*mA*mC*mU-3′
SEQ ID NO: 93
5′-
mG*mC*mG*mC*mUmUmCmUmUmGmAmUmGmGmGmAmAmGmUmCmCmGmGmUmCmUmCmCmUmG
mCmAmCmAmGfAdCdCmAmGmAmUmAmGmAmA*mG*mA*mC*mU-3′
SEQ ID NO: 94
5′-
mG*mC*mG*mC*mUmUmCmUmUmGmAmUmGmGmGmAmAmGmUmCmCmGmGmUmCmUmCmCmUmG
mCmAmCmAmGdAfCdCmAmGmAmUmAmGmAmA*mG*mA*mC*mU-3′
SEQ ID NO: 95
5′-
mG*mC*mG*mC*mUmUmCmUmUmGmAmUmGmGmGmAmAmGmUmCmCmGmGmUmCmUmCmCmUmG
mCmAmCmAmGdAdCfCmAmGmAmUmAmGmAmA*mG*mA*mC*mU-3′
SEQ ID NO: 96
5′-
mG*mC*mG*mC*mUmUmCmUmUmGmAmUmGmGmGmAmAmGmUmCmCmGmGmUmCmUmCmCmUmG
mCmAmCmAmGfAfCfCmAmGmAmUmAmGmAmA*mG*mA*mC*mU-3′
SEQ ID NO: 97
5′
mG*mC*mG*mC*mUmUmCmUmUmGmAmUmGmGmGmAmAmGmUmCmCmGmGmUmCmUmCmCmUmG
mCmAmCmAmGdAfCfCmAmGmAmUmAmGmAmA*mG*mA*mC*mU-3′
SEQ ID NO: 98
5′-
mG*mC*mG*mC*mUmUmCmUmUmGmAmUmGmGmGmAmAmGmUmCmCmGmGmUmCmUmCmCmUmG
mCmAmCmAmGfAfCdCmAmGmAmUmAmGmAmA*mG*mA*mC*mU-3′
SEQ ID NO: 99
AGAAAAGTCCTGGGAAGCTC
SEQ ID NO: 100
GGACTCTGAGTGGTGGTGAT

1. A method of editing a MECP2 polynucleotide comprising a single nucleotide polymorphism (SNP) associated with Rett syndrome, the method comprising contacting the MECP2 polynucleotide with a guide oligonucleotide capable of effecting an adenosine deaminase acting on RNA (ADAR)-mediated adenosine to inosine alteration of the SNP associated with Rett syndrome, thereby editing the MECP2 polynucleotide.

2. (canceled)

3. The method of claim 1, wherein the cell endogenously expresses ADAR.

4-9. (canceled)

10. A method of treating Rett syndrome in a subject in need thereof, the method comprising
contacting the MECP2 polynucleotide in a cell of the subject with a guide oligonucleotide capable of effecting an adenosine deaminase acting on RNA (ADAR)-mediated adenosine to inosine alteration of the SNP associated with Rett syndrome, thereby treating the subject.

11. A method of treating Rett syndrome in a subject in need thereof, the method comprising
contacting the MECP2 polynucleotide in a cell with a guide oligonucleotide capable of effecting an adenosine deaminase acting on RNA (ADAR)-mediated adenosine to inosine alteration of the SNP associated with Rett syndrome, and
administering the cell to the subject, thereby treating the subject.

12-13. (canceled)

14. The method of claim 1, wherein the guide oligonucleotide comprises a nucleic acid sequence complementary to a MECP2 mRNA sequence comprising the SNP associated with Rett syndrome.

15. The method of claim 1, wherein the oligonucleotide further comprises one or more adenosine deaminase acting on RNA (ADAR)-recruiting domains.

16. The method of claim 1, wherein the MECP2 polynucleotide encodes a MECP2 protein comprising a pathogenic amino acid comprising a premature stop codon at positions 255, 168, 294 and/or 270, resulting from the SNP.

17. The method of claim 16, wherein the adenosine to inosine alteration substitutes the pathogenic amino acid with a wild type amino acid, wherein the wild type amino acid at positions 255, 168, 294 and/or 270 is an arginine.

18. (canceled)

19. The method of claim 1, wherein the guide oligonucleotide comprises the structure:
[Aₘ]-X¹-X²-X³—[Bₙ]
wherein each of A and B is a nucleotide;
m and n are each, independently, an integer from 5 to 40;
X¹, X², and X³ are each, independently, a nucleotide, wherein at least one of X¹, X², or X³ is an alternative nucleotide.

20. The method of claim 1, wherein the guide oligonucleotide comprises the structure:
[Aₘ]-X¹-X²-X³—[Bₙ]
wherein each of A and B is a nucleotide;
m and n are each, independently, an integer from 5 to 40;
X¹, X², and X³ are each, independently, a nucleotide, wherein at least one of X¹, X², or X³ has the structure of any one of Formula I-I: 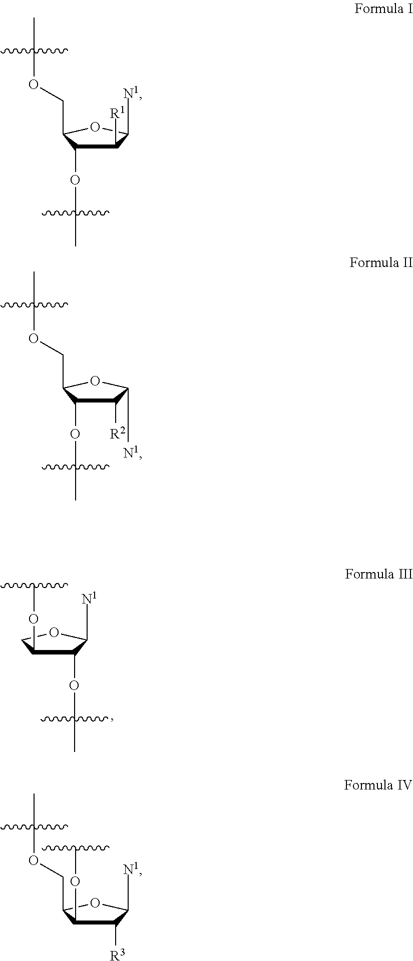
wherein N¹ is hydrogen or a nucleobase;
R¹ is hydroxy, halogen, or C₁-C₆ alkoxy;
R² is hydrogen, hydroxy, halogen, or C₁-C₆ alkoxy; and
R³ is hydrogen, hydroxy, halogen, or C₁-C₆ alkoxy.

21-27. (canceled)

28. The method of claim 20, wherein at least one of X¹, X², and X³ has the structure of Formula I, wherein R¹ is fluoro, hydroxy, or O-methyl, and N¹ is a nucleobase.

29-102. (canceled)

103. The method of claim 1, wherein the guide oligonucleotide comprises the structure:
[Aₘ]-X¹-X²-X³—[Bₙ]
wherein each of A and B is a nucleotide;
m and n are each, independently, an integer from 5 to 40;
X¹, X², and X³ are each, independently, a nucleotide, wherein at least one of X¹, X², and X³ has the structure of any one of Formula XII-XV: 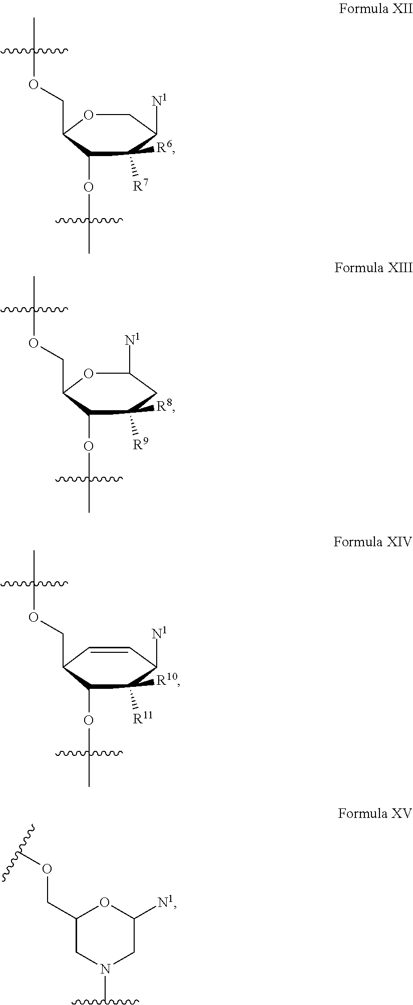
wherein N¹ is hydrogen or a nucleobase;
R⁶ is hydrogen, hydroxy, or halogen;
R⁷ is hydrogen, hydroxy, halogen, or C₁-C₆ alkoxy;
R⁸ is hydrogen or halogen;
R⁹ is hydrogen or hydroxy, halogen, or C₁-C₆ alkoxy;
R¹⁰ Is hydrogen or halogen; and
R¹¹ is hydrogen or hydroxy, halogen, or C₁-C₆ alkoxy.

104-106. (canceled)

107. The method of claim 103, wherein at least one of X¹, X², and X³ has the structure of Formula XIII, in which each of R⁸ and R⁹ is hydrogen.

108-128. (canceled)

129. The method of claim 19, wherein each of [Aₘ] and [Bₙ] comprises at least four terminal phosphorothioate linkages.

130-137. (canceled)

138. The method of claim 19, wherein at least 20% of the nucleotides of [Aₘ] and [Bₙ] combined are 2′-O-methyl-nucleotides.

139-144. (canceled)

145. The method of claim 20, wherein at least one of X¹, X², and X³ has the structure of Formula I, wherein R¹ is fluoro, hydroxy, or methoxy and N¹ is a nucleobase each of X¹, X², and X³ that does not have the structure of Formula I is a ribonucleotide; [Aₘ] and [Bₙ] each comprise at least five terminal 2′-O-methyl-nucleotides and at least four terminal phosphorothioate linkages; and at least 20% of the nucleotides of [Aₘ] and [Bₙ] combined are 2′-O-methyl-nucleotides.

146. (canceled)

147. The method of claim 103, wherein at least of X¹, X², and X³ has the structure of Formula XIII, wherein R⁸ and R⁹ are each hydrogen, and each of X¹, X² and X³ that does not have the structure of Formula XII is a ribonucleotide; [Aₘ] and [Bₙ] each include at least five terminal 2′-O-methyl-nucleotides and at least four terminal phosphorothioate linkages; and at least 20% of the nucleotides of [Aₘ] and [Bₙ] combined are 2′-O-methyl-nucleotides.
